(19) United States
(12) Patent Application Publication
(10) Pub. No.: US 2008/0214807 A1
(43) Pub. Date: Sep. 4, 2008

(54) Substituted Spiro Compounds and their Use for Producing Drugs

(75) Inventors: Hans Schick, Berlin (DE); Robert Frank, Aachen (DE); Melanie Reich, Aachen (DE); Ruth Jostock, Stolberg (DE); Gregor Bahrenberg, Aachen (DE); Fritz Theil, Berlin (DE); Birgitta Henkel, Berlin (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/914,866

(22) PCT Filed: May 17, 2006
(86) PCT No.: PCT/EP06/04651

§ 371 Date: Apr. 18, 2008

(30) Foreign Application Priority Data
May 19, 2005 (DE) 10 2005 023 784.3

(51) Int. Cl. C07D 498/10

(52) U.S. Cl. 544/230; 546/19

(57) ABSTRACT

The present invention relates to substituted spiro compounds, to processes for preparing them, to medicaments comprising these compounds and to the use of these compounds for producing medicaments.

The present invention relates to substituted spiro compounds, to processes for preparing them, to medicaments comprising these compounds and to the use of these compounds for producing medicaments.

The treatment of pain, in particular of neuropathic pain, is of great importance in medicine. Effective pain therapies are in demand across the globe. The urgent need for action to provide patient-friendly and targeted treatment of chronic and non-chronic states of pain, meaning the successful and satisfactory treatment of the patient's pain, is also reflected in the large number of scientific studies which have recently appeared in the field of applied analgesics or basic research in nociception.

A suitable starting point for the treatment of pain, in particular of neuropathic pain, is provided by the subtype 1 vanilloid receptor (VR1/TRPV1) which is often also referred to as a capsaicin receptor. This receptor is stimulated, inter alia, by vanilloids, such as for example capsaicin, heat and protons and plays a central role in the production of pain. In addition, it is important for a large number of further physiological and pathophysiological processes such as, for example, migraine; depression; neurodegenerative diseases; cognitive diseases; panic attacks; epilepsy; coughing; diarrhoea; pruritus; disturbances of the cardiovascular system; eating disorders; medication dependency; medication abuse and in particular urinary incontinence.

An object of the present invention was therefore to provide new compounds which are suitable, in particular, as pharmacological active ingredients in medicaments, preferably in medicaments for the treatment of disturbances or diseases which are transmitted, at least in some cases, by vanilloid receptors 1 (VR1/TRPV1 receptors).

It has surprisingly been found that substituted spiro compounds of general formula I as indicated below are suitable for combating pain and display outstanding affinity to the subtype 1 vanilloid receptor (VR1/TRPV1 receptor) and are therefore suitable, in particular, for the prophylaxis and/or treatment of disturbances or diseases transmitted, at least in some cases, by vanilloid receptors 1 (VR1/TRPV1).

The present invention therefore relates to substituted spiro compounds of general formula I,

wherein
m is equal to 0, 1, 2, 3 or 4,
n is equal to 0, 1 or 2,
R¹ represents a linear or branched, saturated or unsaturated, unsubstituted or at least singly substituted aliphatic radical optionally having at least one heteroatom as chain member;
   an unsubstituted or at least singly substituted, unsaturated or saturated cycloaliphatic radical which optionally has at least one heteroatom as ring member and can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   an unsubstituted or at least singly substituted aryl or heteroaryl radical which can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   or a —C(═S)—NR⁸R⁹ group;
   or if m is unequal to 0 and/or n unequal to 1 and/or R⁵ unequal to H,
   can additionally represent a —C(═O)—NR⁸R⁷ group,
R² represents a linear or branched, saturated or unsaturated, unsubstituted or at least singly substituted aliphatic radical optionally having at least one heteroatom as chain member;
   wherein the substituents of the aliphatic radical can be selected independently of one another from the group consisting of F, Cl, Br, I, —CN, —NO₂, —OH, —SH and —NH₂;
   an unsubstituted or at least singly substituted, unsaturated or saturated cycloaliphatic radical which optionally has at least one heteroatom as ring member and can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   or an unsubstituted or at least singly substituted aryl or heteroaryl radical which can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system;
R³ represents a hydrogen radical,
   a linear or branched, saturated or unsaturated, unsubstituted or at least singly substituted aliphatic radical optionally having at least one heteroatom as chain member,
   wherein the substituents of the aliphatic radical can be selected independently of one another from the group consisting of F, Cl, Br, I, —CN, —NO₂, —OH, —SH and —NH₂;
   an unsubstituted or at least singly substituted, unsaturated or saturated cycloaliphatic radical which optionally has at least one heteroatom as ring member and can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   or an unsubstituted or at least singly substituted aryl or heteroaryl radical which can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system; or
R² and R³ form together with the nitrogen atom connecting them as ring member an unsubstituted or at least singly substituted, unsaturated or saturated cycloaliphatic radical which optionally has at least one further heteroatom as ring member and can be condensed with an unsubstituted or at least singly substituted mono- or polycyclic ring system;
R⁴ represents a halogen radical, a nitro group, a cyano group, an amino group, a hydroxy group, a thiol group, a carboxy group, a formyl group, an —NH—C(═O)—H group, an oxo group (═O), an —NH—R¹⁰ group, an —NR¹¹R¹² group, a —C(═O)—R¹³ group, a —C(═O)—O—R¹⁴ group, an —O—C(═O)—R¹⁵ group, an —NH—C(═O)—R¹⁶ group, an —NR¹⁷—C(═O)—R¹⁸ group, a —C(═O)—NH₂ group, a —C(═O)—NH—R¹⁹ group, a —C(═O)—NR²⁰R²¹ group, an —O—R²² group, an —S—R²³ group, or a linear or branched, saturated or unsaturated, unsubstituted or at least singly substituted aliphatic radical;
R⁵ represents a hydrogen radical, a halogen radical, a nitro group, a cyano group, an amino group, a hydroxy group, a thiol group, a carboxy group, a formyl group, an —NH—C(═O)—H group, an oxo group (═O), an —NH—R¹⁰ group, an —NR¹¹R¹² group, a —C(═O)—R¹³ group, a —C(═O)—O—R¹⁴ group, an —O—C(═O)—R¹⁵ group, an —NH—C(═O)—R¹⁶ group, an —NR¹⁷—C(═O)—R¹⁸ group, a —C(═O)—NH₂ group, a —C(═O)—NH—R¹⁹ group, a —C(═O)—NR²⁰R²¹ group, an —O—R²² group, an —S—R²³ group, a linear or branched, saturated or unsaturated, unsubstituted or at least singly substituted aliphatic radical or an unsubstituted or at least singly substituted aryl or heteroaryl radical which can be bound via a linear or branched, unsubstituted or at least singly substituted alkylene, alkenylene or alkinylene group;
R⁶ and R⁸, independently of one another, each
   represent a linear or branched, saturated or unsaturated, unsubstituted or at least singly substituted aliphatic radical optionally having at least one heteroatom as chain member,
   an unsubstituted or at least singly substituted, unsaturated or saturated cycloaliphatic radical which optionally has at least one heteroatom as ring member and can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system and/or bridged with at least one linear or branched, unsubstituted or at least singly substituted alkylene group,
   an unsubstituted or at least singly substituted aryl or heteroaryl radical which can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   a —C(═O)—R²⁴ group which can be bound via a linear or branched, unsubstituted or at least singly substituted alkylene group,
   or a —C(═O)—O—R²⁵ group which can be bound via a linear or branched, unsubstituted or at least singly substituted alkylene group;
R⁷ and R⁹, independently of one another, each
   represent a hydrogen radical,
   a linear or branched, saturated or unsaturated, unsubstituted or at least singly substituted aliphatic radical optionally having at least one heteroatom as chain member,
   an unsubstituted or at least singly substituted, unsaturated or saturated cycloaliphatic radical which optionally has at least one heteroatom as ring member and can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system and/or bridged with at least one linear or branched, unsubstituted or at least singly substituted alkylene group,
   an unsubstituted or at least singly substituted aryl or heteroaryl radical which can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   a —C(═O)—R²⁴ group which can be bound via a linear or branched, unsubstituted or at least singly substituted alkylene group,
   or a —C(═O)—O—R²⁵ group which can be bound via a linear or branched, unsubstituted or at least singly substituted alkylene group; and
R¹⁰, R¹¹, R¹², R¹³, R¹⁴, R¹⁵, R¹⁶, R¹⁷, R¹⁸, R¹⁹, R²⁰, R²¹, R²², R²³, R²⁴ and R²⁵, independently of one another, each
   represent a linear or branched, saturated or unsaturated, unsubstituted or at least singly substituted aliphatic radical optionally having at least one heteroatom as chain member,
   an unsubstituted or at least singly substituted, unsaturated or saturated cycloaliphatic radical which optionally has at least one heteroatom as ring member and can be bound via a linear or branched, unsubstituted or at least singly substituted alkylene, alkenylene or alkinylene group optionally having at least one heteroatom as chain member and/or condensed with an unsubstituted or at least singly substituted mono- or polycyclic ring system,
   or an unsubstituted or at least singly substituted aryl or heteroaryl radical which can be bound via a linear or branched, unsubstituted or at least singly substituted alkylene, alkenylene or alkinylene group optionally having at least one heteroatom as chain member,
      in each case optionally in the form of one of their pure stereoisomers, in particular enantiomers or diastereomers, their racemates or in the form of a mixture of stereoisomers, in particular of enantiomers and/or diastereomers, in any desired mixing ratio, or respectively in the form of corresponding salts, or respectively in the form of corresponding solvates.

Preferably, the substituents of the above-mentioned aliphatic radicals in the position of the substituents R¹ to R²⁵ can be selected independently of one another from the group consisting of F, Cl, Br, I, —CN, —NO₂, —OH, —SH and —NH₂.

Preferably, the radicals R⁵, R² and R³ can have alkylene, alkenylene or alkinylene groups which can be respectively substituted with substituents selected independently of one another from the group consisting of F, Cl, Br, —OH, —SH, —NH₂, —CN, —NO₂ and phenyl; wherein the phenyl radical can be substituted with 1, 2, 3, 4 or 5 substituents selected independently of one another from the group consisting of F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl.

Preferably, the radicals R² and R³ form together with the nitrogen atom connecting them a heterocycloaliphatic radical which can be substituted with substituents selected independently of one another from the group consisting of oxo (═O), thioxo (═S), F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —NH—C₁₋₅ alkyl, —N(C₁₋₅ alkyl)₂, —NH—C(═O)—O—C₁₋₅ alkyl, —S(═O)₂—C₁₋₅ alkyl, —S(═O)₂ phenyl, —NH—S(═O)₂—C₁₋₁₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the radicals pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents selected independently of one another from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl.

Aliphatic radicals include in the sense of the present invention acyclic saturated or unsaturated hydrocarbon radicals which can be branched or straight chained and unsubstituted or singly substituted or multiply substituted, for example 1, 2, 3, 4, 5, 6, 7, 8 or 9 times, by the same or different substituents, containing 1 to 20 (i.e. 1, 2, 3, 4, 5, 6, 7, 8, 9, 10, 11, 12, 13, 14, 15, 16, 17, 18, 19 or 20), preferably 1 to 12 (i.e. 1, 2, 3, 4, 5, 6, 7, 8, 9, 10, 11 or 12), particularly preferably 1 to 6 (i.e. 1, 2, 3, 4, 5 or 6) carbon atoms, i.e. C₁₋₂₀, C₁₋₁₂, C₁₋₆ alkyls, C₂₋₂₀, C₂₋₁₂, C₂₋₆ alkenyls and C₂₋₂₀, C₂₋₁₂, C₂₋₆ alkinyls. Alkenyls have at least one C—C double bond and alkinyls at least one C—C triple bond. Examples include aliphatic radicals selected from the group consisting of methyl, ethyl, n-propyl, isopropyl, n-butyl, isobutyl, sec-butyl, tert-butyl, n-pentyl, isopentyl, neo-pentyl, n-hexyl, 2-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, ethenyl (vinyl), ethinyl, propenyl (—CH₂CH═CH₂, —CH═CH—CH₃, —C(═CH₂)—CH₃), 2-methylpropenyl, propinyl (—CH₂—C≡CH, —C≡C—CH₃), butenyl, butinyl, pentenyl, pentinyl, hexenyl, hexinyl, octenyl and octinyl.

The above-mentioned aliphatic radicals can preferably have 1, 2 or 3 heteroatoms selected independently of one another from the group comprising oxygen, sulphur and nitrogen, including —N(H)— and —N(C₁₋₆ alkyl), as chain members.

Examples of aliphatic radicals having 1, 2 or 3 heteroatoms include —(CH₂)—(CH₂)—O—CH₃, —(CH₂)—(CH₂)—(CH₂)—O—CH₃, —(CH₂)—(CH₂)—(CH₂)—N(C₂H₅)—(C₂H₅), —(CH₂)—(CH₂)—S—CH₃, —(CH₂)—(CH₂)—(CH₂)—S—CH₃, —(CH₂)—(CH₂)—(CH₂)—N(CH₃)—(CH₃) and —(CH₂)—O—CH₃.

In relation to aliphatic radicals, the term “substituted”—unless otherwise defined—refers in the sense of the present invention to the single or multiple substitution, preferably the single, double, triple, quadruple, quintuple, sextuple, septuple, octuple or nonuple substitution, of one or more hydrogen atoms by, for example, F, Cl, Br, I, —CN, —NO₂, —OH, —SH and —NH₂, wherein the multiple substitution is carried out either on different or on identical atoms several times, for example twice or three times, for example three times on the same carbon atom as in the case of —CF₃ or —CH₂CF₃ or at various locations as in the case of —CH(OH)—CH═CCl—CH₂Cl. The multiple substitution can be carried out with the same or with different substituents. Preferred substituted aliphatic radicals include —CH₂—Cl, —CH₂—Br, —CH₂—CH₂—Cl, —CH₂—CH₂—Br, —CH₂—CH₂—CH₂—Br and —CH₂—CH₂—CH₂—Cl.

Cycloaliphatic radicals in the sense of the present invention are cyclic saturated or unsaturated hydrocarbon radicals containing 3, 4, 5, 6, 7, 8, 9, 10, 11, 12, 13, 14, 15 or 16, preferably 3, 4, 5, 6, 7 or 8 carbon atoms, wherein each radical can be unsubstituted or singly or multiply substituted, for example 1, 2, 3, 4, 5, 6, 7, 8 or 9 times, by the same or different substituents. Cycloaliphatic radicals can preferably have 1, 2, 3, 4 or 5 heteroatoms selected independently of one another from the group consisting of oxygen, nitrogen (NH) and sulphur as ring members.

Examples of cycloaliphatic radicals, which can optionally be bridged with 1 or 2 linear or branched C₁₋₅ alkylene groups and condensed with a mono- or polycyclic ring system, include cyclopropyl, cyclobutyl, cyclopentyl, cyclohexyl, cyclooctyl, cyclononyl, cyclodecyl, cycloundecyl, cyclododecyl, cycloheptyl, cyclopentenyl, cyclohexenyl, cycloheptenyl, [6,6]-dimethyl-[3.1.1]-bicycloheptyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl, (1,2,3,4)-tetrahydroquinazolinyl, (1,3,4,5)-tetrahydropyrido[4,3-b]indolyl, (3,4)-dihydro-1H-isoquinolinyl, (1,3,4,9)-tetrahydro-[b]-carbolinyl, imidazolidinyl and (1,3)-thiazolidinyl.

A mono- or polycyclic ring system refers in the sense of the present invention to mono- or polycyclic hydrocarbon radicals which are saturated or unsaturated and can optionally have 1, 2, 3, 4 or 5 heteroatom(s) as ring member(s) which are selected independently of one another from the group consisting of oxygen, nitrogen and sulphur. A mono- or polycyclic ring system of this type can, for example, be condensed (anellated) with an aryl radical or a heteroaryl radical.

If a polycyclic ring system such as, for example, a bicyclic ring system is present, the various rings can, each independently of one another, have a differing degree of saturation, i.e. be saturated or unsaturated. Preferably, a polycyclic ring system is a bicyclic ring system.

Examples of aryl radicals which are condensed with a mono- or polycyclic ring system include [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl.

In relation to cycloaliphatic radicals and mono- or polycyclic ring systems, the term “substituted”—unless otherwise defined—refers in the sense of the present invention to the single or multiple substitution, preferably the single, double, triple, quadruple, quintuple, sextuple, septuple, octuple or nonuple substitution, of one or more hydrogen atoms by, for example, oxo (═O), thioxo (═S), F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)—benzo[b]furanyl, —O-phenyl, —O-benzyl, phenyl and benzyl, wherein in each case the cyclic portion of the radicals pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents selected independently of one another from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl. The multiple substitution can be carried out either on different or on identical atoms several times, for example twice or three times. The multiple substitution can be carried out with the same or with different substituents.

The expression aryl radical refers for the purposes of the present invention preferably to a radical which is selected from the group comprising phenyl, naphthyl, phenanthrenyl and anthracenyl and is unsubstituted or singly or multiply substituted by the same or different substituents. Preferably, the aryl is an unsubstituted or singly substituted phenyl, 1-naphthyl or 2-naphthyl or a phenyl, 1-naphthyl or 2-naphthyl substituted several times, for example twice, three, four or five times, by the same or different substituents.

Heteroaryl radicals in the sense of the present invention are heterocycles which are heteroaromatic. Heteroaryl radicals have preferably 5 to 14 members, i.e. 5, 6, 7, 8, 9, 10, 11, 12, 13 or 14 members, and have preferably 1, 2, 3, 4 or 5 heteroatoms selected independently of one another from the group comprising oxygen, nitrogen and sulphur. Each heteroaryl radical can be unsubstituted or singly substituted or substituted several times, for example twice, three, four or five times, by the same or different substituents.

Examples of heteroaryl radicals in the sense of the present invention include thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl, benzothiazolyl, benzo[2,1,3]thiadiazolyl, [1,2,3]-benzothiadiazolyl, [2,1,3]-benzoxadiazolyl and [1,2,3]-benzoxadiazolyl.

In relation to aryl and heteroaryl radicals, in the sense of the present invention, “substituted” refers to the single or multiple, for example the single, double, triple, quadruple or quintuple, substitution of one or more hydrogen atoms of the ring system by suitable substituents. Insofar as these suitable substituents are not defined in relation to aryl or heteroaryl radicals elsewhere in the description or in the claims, suitable substituents include F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the radicals pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents selected independently of one another from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl. The multiple substitution is carried out with the same or with different substituents.

The above-mentioned linear or branched alkylene, alkenylene or alkinylene groups preferably have 1 to 5 carbon atoms, i.e. the groups are C₁₋₅ alkylene, C₂₋₅ alkenylene or C₂₋₅ alkinylene groups which can be respectively unsubstituted or substituted with 1, 2, 3, 4 or 5 substituents selected independently of one another from the group consisting of F, Cl, Br, —OH, —SH, —NH₂, —CN, —NO₂ and phenyl.

The above-mentioned alkylene, alkenylene or alkinylene groups optionally each have 1 or 2 heteroatom(s) selected from the group consisting of oxygen, nitrogen, i.e.—N(H)— and —N(C₁₋₆ alkyl)- and sulphur as chain member(s).

Preferably, alkylene groups can be selected from the group consisting of —(CH₂)—, —(CH₂)₂—, —C(H)(CH₃)—, —C(CH₃)₂—, —(CH₂)₃—, —(CH₂)₄—, —(CH₂)₅—, —C(H)(CH₃)—(CH₂)—, —C(H)(C₂H₅)—(CH₂)—, —C(phenyl)₂-, —C(H)(phenyl)-, —(CH₂)—O—, —(CH₂)—N(CH₃)—, —(CH₂)—S—, —(CH₂)—(CH₂)—N(CH₃)— and —(CH₂)—(CH₂)—N(C₂H₅)—.

A person skilled in the art will understand that for m is equal to 0, the following general formula Ic is obtained:

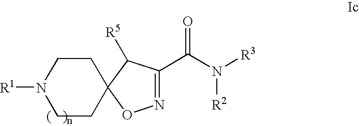

Also preferred are substituted Spiro compounds of the above-indicated general formula I, wherein

n is equal to 1;

and m and R¹ to R⁴² are each as defined above, in each case optionally in the form of one of their pure stereoisomers, in particular enantiomers or diastereomers, their racemates or in the form of a mixture of stereoisomers, in particular of enantiomers and/or diastereomers, in any desired mixing ratio, or respectively in the form of corresponding salts, or respectively in the form of corresponding solvates.

A person skilled in the art will understand that for n is equal to 1, the following general formula Id is obtained:

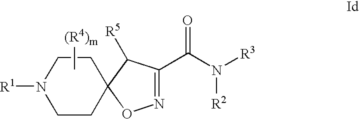

Particularly preferred are also substituted spiro compounds of the above-indicated general formula I, wherein
m is equal to 0, 1, 2, 3 or 4;
n is equal to 1;
R¹ represents a radical selected from the group consisting of methyl, ethyl, n-propyl, isopropyl, n-butyl, sec-butyl, isobutyl, tert-butyl, n-pentyl, sec-pentyl, n-hexyl, n-heptyl and n-octyl;
   represents a (hetero)cycloaliphatic radical selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl and thiomorpholinyl;
   represents a radical selected from the group consisting of indolyl, isoindolyl, benzo[b]furanyl, benzo[b]thiophenyl, indazolyl, quinazolinyl, quinolinyl, isoquinolinyl, benzimidazolinyl, benzoxazolyl, benzisoxazolyl and benzothiazolyl, wherein the radical can in each case optionally be substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —NH—S(═O)₂—CH₃, —NH—S(═O)₂—C₂H₅, —S(═O)₂—NH—CH₃, —S(═O)₂—NH—C₂H₅;
   represents a —C(═S)—NR⁸R⁹ group;

or represents —(CHR²⁶)—R²⁹; —(CHR²⁶)—(CHR²⁷)—R²⁹; —(CHR²⁶)—(CH R²¹)—(CHR²¹)—R²⁹;
or if m is unequal to 0 and/or n unequal to 1 and/or R⁵ unequal to H, can additionally represent a —C(═O)—NR⁶R⁷ group,
R² represents a radical selected from the group consisting of methyl, ethyl, n-propyl, isopropyl, n-butyl, sec-butyl, isobutyl, tert-butyl, n-pentyl, sec-pentyl n-hexyl and n-heptyl;
   represents a radical selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl and thiomorpholinyl, wherein the radical can in each case be substituted with 1 or 2 substituents selected independently of one another from the group consisting of pyridinyl, pyridazinyl, phenyl and benzyl, wherein in each case the cyclic portion of the radicals pyridinyl, pyridazinyl, phenyl and benzyl can be substituted with 1, 2, 3, 4 or 5 substituents selected independently of one another from the group consisting of —CF₃, F, Cl, Br, methyl, ethyl, n-propyl, isopropyl, n-butyl, sec-butyl, isobutyl, tert-butyl, —O—CH₃ and —O—C₂H₅;
   represents a radical selected from the group consisting of phenyl, naphthyl, thiophenyl, furanyl, pyrrolyl, pyrazolyl, pyrazinyl and pyridinyl, wherein the radical can in each case optionally be substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —S(═O)₂—CH₃, —S(═O)₂—C₂H₅, —NH—S(═O)₂—CH₃, —NH—S(═O)₂—C₂H₅, —S(═O)₂—NH—CH₃ and —S(═O)₂—NH—C₂H₅;
   or represents —(CHR³⁰)—R³³; —(CHR³¹)—(CHR³¹)—R³³ or —(CHR³⁰)—(CHR³)—(CHR³²) R³³;
R³ represents a hydrogen radical; or
R² and R³ form together with the nitrogen atom connecting them as ring member a radical selected from the group consisting of pyrrolidinyl, piperidinyl, piperazinyl, 3-methyl piperazinyl, 2-methylpiperazinyl, (3,5)-dimethyl piperazinyl, (2,6)-dimethylpiperazinyl, morpholinyl and thiomorpholinyl, wherein the radical can in each case be substituted with 1 or 2 substituents selected independently of one another from the group consisting of —C(═O)—O—CH₃, —C(═O)—O—C₂H₅, —C(═O)—O—C(CH₃)₃, —C(═O)—CH₃, —C(═O)—C₂H₅, —C(═O)—C(CH₃)₃, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, pyridinyl, pyridazinyl, phenyl and benzyl, wherein in each case the cyclic portion of the radicals pyridinyl, —S(═O)₂ phenyl, pyridazinyl, phenyl and benzyl can be substituted with 1, 2, 3, 4 or 5 substituents selected independently of one another from the group consisting of —CF₃, F, Cl, Br, methyl, ethyl, n-propyl, isopropyl, n-butyl, sec-butyl, isobutyl, tert-butyl, —O—CH₃ and —O—C₂H₅;
R⁴ represents a radical selected from the group consisting of methyl, ethyl, n-propyl, isopropyl, n-butyl, sec-butyl, isobutyl, tert-butyl, n-pentyl, sec-pentyl, n-hexyl and n-heptyl,
R⁵ represents a hydrogen radical,
   a radical selected from the group consisting of methyl, ethyl, n-propyl, isopropyl, n-butyl, sec-butyl, isobutyl, tert-butyl, n-pentyl, sec-pentyl, n-hexyl and n-heptyl,
   represents a phenyl radical which can be respectively substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl and tert-butyl;
   or represents —(CH₂)—R³⁴;
R⁶ and R⁸, independently of one another, each represent a radical selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl, 2-methyl-1-propenyl, ethinyl, 1-propinyl, 2-propinyl, 1-butinyl, 2-butinyl and 3-butinyl, wherein the radical can in each case optionally be substituted with 1, 2, 3, 4 or 5 substituents selected independently of one another from the group consisting of F, Cl and Br;
   represent a radical selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl;
   represent a radical selected from the group consisting of phenyl, naphthyl, thiophenyl, furanyl, pyrrolyl, pyrazolyl, pyranyl, triazolyl, pyridinyl, imidazolyl, thiazolyl, oxazolyl, isoxazolyl, pyridazinyl, pyrazinyl and pyrimidinyl, wherein the radical can in each case optionally be substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the radicals pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents selected independently of one another from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
   represent —(CHR³⁶)—C(═O)—O—R²⁵ or —(CHR³⁶)—(CH₂)—C(═O)—O—R²⁵;
   or represent —(CR³⁷R³⁸)—R⁴¹, —(CR³⁷R³⁸)—(CHR³⁹)—R⁴¹, —(CR³⁷R³⁸)—(CHR³⁹)—O—R⁴¹, —(CR³⁷R³⁸)—(CHR³⁹)—(CHR⁴⁰)—R⁴¹, —(CR³⁷R³⁸)—(CHR³⁹)—(CHR⁴⁰)—O—R⁴¹, (CR³⁷R³⁸)—(CHR³⁹)—(CHR⁴⁰)—N(CH₃)—R⁴¹ or —(CR³⁷R³⁸)—(CHR³⁹)—(CHR⁴⁰)—N(C₂H₅)—R⁴¹;
R⁷ and R⁹ each represent a hydrogen radical;
R²⁵ represents a radical selected from the group consisting of methyl, ethyl, n-propyl, isopropyl, n-butyl, sec-butyl, isobutyl and tert-butyl;
   R²⁶, R²⁷, R²⁸, R³⁰R³¹, R³², R³⁶, R³⁷, R³⁸, R³⁹ and R⁴⁰, independently of one another, each
   represent a hydrogen radical;
   represent a radical selected from the group consisting of methyl, ethyl, n-propyl, isopropyl, n-butyl, sec-butyl, isobutyl and tert-butyl;

or represent a phenyl radical which can be substituted with 1, 2, 3, 4 or 5 substituents selected independently of one another from the group consisting of F, Cl, Br, I, —CN, —CF₃, —O—CH₃, —O—C₂H₅, —NO₂, —O—CF₃, methyl, ethyl, n-propyl, isopropyl, n-butyl, sec-butyl, isobutyl and tert-butyl;
R²⁹ and R³³, independently of one another, each
   represent a radical selected from the group consisting of phenyl, naphthyl, thiophenyl, furanyl, pyrrolyl, pyrazolyl, pyrazinyl and pyridinyl, wherein the radical can in each case optionally be substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —S(═O)₂—CH₃, —S(═O)₂—C₂H₅, —NH—S(═O)₂—CH₃, —NH—S(═O)₂—C₂H₅, —S(═O)₂—NH—CH₃ and —S(═O)₂—NH—C₂H₅;
R³⁴ represents a phenyl radical which can be substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl and tert-butyl; and
R⁴¹ represents a radical selected from the group consisting of methyl, ethyl, n-propyl, isopropyl, n-butyl, sec-butyl, isobutyl and tert-butyl;
   represents a radical selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl and dithiolanyl;
   or represents a radical selected from the group consisting of phenyl, naphthyl, (1,3)-benzodioxolyl, (1,4)-benzodioxanyl, thiophenyl and furanyl, wherein the radical can in each case optionally be substituted with 1, 2, 3, 4 or 5 substituents selected independently of one another from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅ and —C(═O)—C(CH₃)₃;
      in each case optionally in the form of one of their pure stereoisomers, in particular enantiomers or diastereomers, their racemates or in the form of a mixture of stereoisomers, in particular of enantiomers and/or diastereomers, in any desired mixing ratio, or respectively in the form of corresponding salts, or respectively in the form of corresponding solvates.

Most particularly preferred are substituted spiro compounds of the above-indicated general formula I, wherein
m is equal to 0;
n is equal to 1;
R¹ represents a radical selected from the group consisting of benzimidazolinyl, benzoxazolyl, benzisoxazolyl and benzothiazolyl, wherein the radical can in each case be substituted with 1, 2, 3, 4 or 5 substituents selected independently of one another from the group consisting of F, Cl, Br, —CF₃, —O—CF₃, —S—CF₃, methyl, ethyl, n-propyl, isopropyl, n-butyl, sec-butyl, isobutyl and tert-butyl; or
   represents a —C(═S)—NR⁸R⁹ group;
R² represents a radical selected from the group consisting of phenyl, naphthyl and pyridinyl, wherein the radical can in each case be substituted with 1, 2 or 3 substituents selected independently of one another from the group consisting of F, Cl, Br, —OH, —O—CH₃, —O—C₂H₅, —NH—S(═O)₂—CH₃ and —NH—S(═O)₂—C₂H₅;
   represents a piperazinyl radical which on a nitrogen atom can be substituted with a substituent selected from the group consisting of pyridinyl, pyridazinyl, phenyl and benzyl, wherein in each case the cyclic portion of the substituents pyridinyl, pyridazinyl, phenyl and benzyl can be substituted with 1, 2 or 3 substituents selected independently of one another from the group consisting of —CF₃, F, Cl, Br, methyl, ethyl, n-propyl, isopropyl, n-butyl, sec-butyl, isobutyl and tert-butyl;
   or represents —(CHR³⁰)—R³³; —(CHR³⁰)—(CHR³¹)—R³³ or —(CHR³⁰)—(CHR³¹)—(CHR³²)—R³³;
R³ represents a hydrogen radical;
   or
R² and R³ form together with the connecting nitrogen atom as ring member a radical selected from the group consisting of 3-methylpiperazinyl, 2-methylpiperazinyl, (3,5)-dimethyl piperazinyl, (2,6)-dimethylpiperazinyl and piperazinyl which on a nitrogen atom can be substituted with a substituent selected from the group consisting of pyridinyl, pyridazinyl, phenyl and benzyl, wherein in each case the cyclic portion of the substituents pyridinyl, pyridazinyl, phenyl and benzyl can be substituted with 1, 2 or 3 substituents selected independently of one another from the group consisting of —CF₃, F, Cl, Br, methyl, ethyl, n-propyl, isopropyl, n-butyl, sec-butyl, isobutyl and tert-butyl;
R⁵ represents a hydrogen radical,
R⁶ and R⁸, independently of one another, each represent a radical selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl and 2-methyl-1-propenyl, wherein the radical can in each case optionally be substituted with 1, 2, 3, 4 or substituents selected independently of one another from the group consisting of F, Cl and Br;
   represent a radical selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl and adamantyl;
   represent a radical selected from the group consisting of phenyl, naphthyl and pyridinyl, wherein the radical can in each case optionally be substituted with 1, 2, 3, 4 or 5 substituents selected independently of one another from the group consisting of —SF₅, F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl, wherein in each case the cyclic portion of the radicals cyclopentyl, cyclohexyl, —O-phenyl, —O-benzyl and phenyl can be substituted with 1, 2, 3, 4 or 5 substituents selected independently of one another from the group consisting of F, Cl, Br, methyl, ethyl, n-propyl, isopropyl, n-butyl, sec-butyl, isobutyl and tert-butyl;
   represent —(CH R³⁶)—C(═O)—O—R²⁵ or —(CHR³⁶)—(CH₂)—C(═O)—O—R²⁵;
   or represent —(CR³⁷R³⁸)—R⁴¹, —(CR³⁷R³⁸)—(CHR³⁹)—R⁴¹—(CR³⁷R³⁸)—(CHR³⁹) O R⁴¹, —(CR³⁷R³⁸)—(CHR³⁹)—(CHR⁴⁰)—R⁴¹, —(CR³⁷R³³)—(CHR³⁹)—(CHR⁴⁰)—O—R⁴¹, (CR³⁷R³⁸)—(CHR³⁹)—(CHR⁴⁰)—N(CH₃)—R⁴¹ or —(CR³⁷R³³)—(CHR³⁹)—(CHR⁴⁰)—N(C₂H₅)—R⁴¹;
R⁷ and R⁹ each represent a hydrogen radical;
R²⁵ represents a radical selected from the group consisting of methyl, ethyl, n-propyl, isopropyl, n-butyl, sec-butyl, isobutyl and tert-butyl;
R³⁰, R³¹, R³², R³⁹ and R⁴⁰ each represent a hydrogen radical;
R³³ represents a radical selected from the group consisting of phenyl, naphthyl, thiophenyl and furanyl, wherein the radical can in each case optionally be substituted with 1, 2, 3, 4 or 5 substituents selected independently of one another from the group consisting of —CF₃, F, Cl, Br, —OH, —O—CH₃, —O—C₂H₅, methyl, ethyl, n-propyl, isopropyl, n-butyl, sec-butyl, isobutyl, tert-butyl, —NH—S(═O)₂—CH₃ and —NH—S(═O)₂—C₂H₅;
R³⁶ represents a hydrogen radical
   or a radical selected from the group consisting of methyl, ethyl, n-propyl, isopropyl, n-butyl, sec-butyl, isobutyl and tert-butyl;

R³⁷ and R³⁸, independently of one another, each
represent a hydrogen radical;
represent a radical selected from the group consisting of methyl, ethyl, n-propyl, isopropyl, n-butyl, sec-butyl, isobutyl and tert-butyl;
or represent a phenyl radical;

R⁴¹ represents a radical selected from the group consisting of methyl, ethyl, n-propyl, isopropyl, n-butyl, sec-butyl, isobutyl and tert-butyl;
represents a radical selected from the group consisting of tetrahydrofuranyl, tetrahydrothiophenyl, pyrrolidinyl, morpholinyl, piperidinyl and piperazinyl;
or represents a radical selected from the group consisting of phenyl, naphthyl, (1,3)-benzodioxolyl, (1,4)-benzodioxanyl, thiophenyl and furanyl, wherein the radical can in each case optionally be substituted with 1, 2, 3, 4 or 5 substituents selected independently of one another from the group consisting of F, Cl, Br, —OH, —O—CH₃, —O—C₂H₅, methyl, ethyl, n-propyl, isopropyl, n-butyl, sec-butyl, isobutyl and tert-butyl;
in each case optionally in the form of one of their pure stereoisomers, in particular enantiomers or diastereomers, their racemates or in the form of a mixture of stereoisomers, in particular of enantiomers and/or diastereomers, in any desired mixing ratio, or respectively in the form of corresponding salts, or respectively in the form of corresponding solvates.

The present invention further relates to substituted spiro compounds selected from the group consisting of
[1] 3-[4-(3-chloropyridin-2-yl)piperazine-1-carbonyl]-1-oxa-2,8-diazaspiro[4.5]dec-2-ene-8-carboxylic acid-(2-fluorophenyl)amide
[2] 1-oxa-2,8-diazaspiro[4.5]dec-2-ene-3,8-dicarboxylic acid-3-[(5-chloropyridin-2-yl)amide]-8-[(2-fluorophenyl)amide]
[3] 1-oxa-2,8-diazaspiro[4.5]dec-2-ene-3,8-dicarboxylic acid-3-(4-chlorobenzylamide)-8-[(3-fluorophenyl)amide]
[4] 3-[4-(3-chloropyridin-2-yl)piperazine-1-carbonyl]-1-oxa-2,8-diazaspiro[4.5]dec-2-ene-8-carboxylic acid-(3-fluorophenyl)amide
[5] 1-oxa-2,8-diazaspiro[4.5]dec-2-ene-3,8-dicarboxylic acid-3-[(5-chloropyridin-2-yl)amide]-8-[(3-fluorophenyl)amide]
[6] 1-oxa-2,8-diazaspiro[4.5]dec-2-ene-3,8-dicarboxylic acid-8-[(2-chlorophenyl)amide]-3-(3,4-dimethoxybenzylamide)
[7] 1-oxa-2,8-diazaspiro[4.5]dec-2-ene-3,8-dicarboxylic acid-3-(4-chlorobenzylamide)-8-[(2-chlorophenyl)amide]
[8] 3-[4-(3-chloropyridin-2-yl)piperazine-1-carbonyl]-1-oxa-2,8-diazaspiro[4.5]dec-2-ene-8-carboxylic acid-(2-chlorophenyl)amide
[9] 1-oxa-2,8-diazaspiro[4.5]dec-2-ene-3,8-dicarboxylic acid-8-[(2-chlorophenyl)amide]-3-[(5-chloropyridin-2-yl)amide]
[10] 1-oxa-2,8-diazaspiro[4.5]dec-2-ene-3,8-dicarboxylic acid-8-[(3-bromophenyl)amide]-3-(3,4-dimethoxybenzylamide)
[11] 3-[4-(3-chloropyridin-2-yl)piperazine-1-carbonyl]-1-oxa-2,8-diazaspiro[4.5]dec-2-ene-8-carboxylic acid-(3-bromophenyl)amide
[12] 1-oxa-2,8-diazaspiro[4.5]dec-2-ene-3,8-dicarboxylic acid-8-[(3-bromophenyl)amide]-3-[(5-chloropyridin-2-yl)amide]
[13] 1-oxa-2,8-diazaspiro[4.5]dec-2-ene-3,8-dicarboxylic acid-3-(3,4-dimethoxybenzylamide)-8-[(2-methoxyphenyl)amide]
[14] 1-oxa-2,8-diazaspiro[4.5]dec-2-ene-3,8-dicarboxylic acid-3-(4-chlorobenzylamide)-8-[(2-methoxyphenyl)amide]
[15] 3-[4-(3-chloropyridin-2-yl)piperazine-1-carbonyl]-1-oxa-2,8-diazaspiro[4.5]dec-2-ene-8-carboxylic acid-(2-methoxyphenyl)amide
[16] 1-oxa-2,8-diazaspiro[4.5]dec-2-ene-3,8-dicarboxylic acid-3-(3,4-dimethoxybenzylamide)-8-[(3-methoxyphenyl)amide]
[17] 3-[4-(3-chloropyridin-2-yl)piperazine-1-carbonyl]-1-oxa-2,8-diazaspiro[4.5]dec-2-ene-8-carboxylic acid-(3-methoxyphenyl)amide
[18] 1-oxa-2,8-diazaspiro[4.5]dec-2-ene-3,8-dicarboxylic acid-3-[(5-chloropyridin-2-yl)amide]-8-[(3-methoxyphenyl)amide]
[19] 3-[4-(3-chloropyridin-2-yl)piperazine-1-carbonyl]-1-oxa-2,8-diazaspiro[4.5]dec-2-ene-8-carboxylic acid-(4-phenoxyphenyl)amide
[20] 1-oxa-2,8-diazaspiro[4.5]dec-2-ene-3,8-dicarboxylic acid-8-[(2-chloro-5-trifluoromethylphenyl)amide]-3-(3,4-dimethoxybenzylamide)
[21] 1-oxa-2,8-diazaspiro[4.5]dec-2-ene-3,8-dicarboxylic acid-3-(4-chlorobenzylamide)-8-[(2-chloro-5-trifluoromethylphenyl)amide]
[22] 3-[4-(3-chloropyridin-2-yl)piperazine-1-carbonyl]-1-oxa-2,8-diazaspiro[4.5]dec-2-ene-8-carboxylic acid-(2-chloro-5-trifluoromethylphenyl)amide
[23] 1-oxa-2,8-diazaspiro[4.5]dec-2-ene-3,8-dicarboxylic acid-3-[(5-chloropyridin-2-yl)amide]-8-[(2-chloro-5-trifluoromethylphenyl)amide]
[24] 1-oxa-2,8-diazaspiro[4.5]dec-2-ene-3,8-dicarboxylic acid-8-[(4-chloro-2-trifluoromethylphenyl)amide]-3-(3,4-dimethoxybenzylamide)
[25] 3-[4-(3-chloropyridin-2-yl)piperazine-1-carbonyl]-1-oxa-2,8-diazaspiro[4.5]dec-2-ene-8-carboxylic acid-(4-chloro-2-trifluoromethylphenyl)amide
[26] 1-oxa-2,8-diazaspiro[4.5]dec-2-ene-3,8-dicarboxylic acid-3-[(5-chloropyridin-2-yl)amide]-8-[(4-chloro-2-trifluoromethylphenyl)amide]
[27] 1-oxa-2,8-diazaspiro[4.5]dec-2-ene-3,8-dicarboxylic acid-8-[(4-chloro-3-trifluoromethylphenyl)amide]-3-(3,4-dimethoxybenzylamide)
[28] 3-[4-(3-chloropyridin-2-yl)piperazine-1-carbonyl]-1-oxa-2,8-diazaspiro[4.5]dec-2-ene-8-carboxylic acid-(4-chloro-3-trifluoromethylphenyl)amide
[29] 1-oxa-2,8-diazaspiro[4.5]dec-2-ene-3,8-dicarboxylic acid-3-[(5-chloropyridin-2-yl)amide]-8-[(4-chloro-3-trifluoromethylphenyl)amide]
[30] 1-oxa-2,8-diazaspiro[4.5]dec-2-ene-3,8-dicarboxylic acid-3-[(2-tert-butyl-6-methylphenyl)amide]-8-(4-chlorobenzylamide)
[31] 3-[4-(3-chloropyridin-2-yl)piperazine-1-carbonyl]-1-oxa-2,8-diazaspiro[4.5]dec-2-ene-8-carboxylic acid-(2-tert-butyl-6-methylphenyl)amide
[32] 1-oxa-2,8-diazaspiro[4.5]dec-2-ene-3,8-dicarboxylic acid-3-(3,4-dimethoxybenzylamide)-8-[(4-trifluoromethoxyphenyl)amide]
[33] 1-oxa-2,8-diazaspiro[4.5]dec-2-ene-3,8-dicarboxylic acid-3-(4-chlorobenzylamide)-8-[(4-trifluoromethoxyphenyl)amide]
[34] 3-[4-(3-chloropyridin-2-yl)piperazine-1-carbonyl]-1-oxa-2,8-diazaspiro[4.5]dec-2-ene-8-carboxylic acid-(4-trifluoromethoxyphenyl)amide
[35] 1-oxa-2,8-diazaspiro[4.5]dec-2-ene-3,8-dicarboxylic acid-3-[(5-chloropyridin-2-yl)amide]-8-[(4-trifluoromethoxyphenyl)amide]
[36] 1-oxa-2,8-diazaspiro[4.5]dec-2-ene-3,8-dicarboxylic acid-3-(3,4-dimethoxybenzylamide)-8-(phenethylamide)
[37] 1-oxa-2,8-diazaspiro[4.5]dec-2-ene-3,8-dicarboxylic acid-3-(4-chlorobenzylamide)-8-(phenethylamide)
[38] 3-[4-(3-chloropyridin-2-yl)piperazine-1-carbonyl]-1-oxa-2,8-diazaspiro[4.5]dec-2-ene-8-carboxylic acid phenethylamide
[39] 1-oxa-2,8-diazaspiro[4.5]dec-2-ene-3,8-dicarboxylic acid-3-(3,4-dimethoxybenzylamide)-8-phenylamide
[40] 3-[4-(3-chloropyridin-2-yl)piperazine-1-carbonyl]-1-oxa-2,8-diazaspiro[4.5]dec-2-ene-8-carboxylic acid phenylamide
[41] 1-oxa-2,8-diazaspiro[4.5]dec-2-ene-3,8-dicarboxylic acid-3-[(5-chloropyridin-2-yl)amide]-8-phenylamide
[42] 1-oxa-2,8-diazaspiro[4.5]dec-2-ene-3,8-dicarboxylic acid-3-(4-chlorobenzylamide)-8-m-tolylamide
[43] 3-[4-(3-chloropyridin-2-yl)piperazine-1-carbonyl]-1-oxa-2,8-diazaspiro[4.5]dec-2-ene-8-carboxylic acid-m-tolylamide
[44] 1-oxa-2,8-diazaspiro[4.5]dec-2-ene-3,8-dicarboxylic acid-3-(3,4-dimethoxybenzylamide)-8-[(4-fluorophenyl)amide]
[45] 3-[4-(3-chloropyridin-2-yl)piperazine-1-carbonyl]-1-oxa-2,8-diazaspiro[4.5]dec-2-ene-8-carboxylic acid-(4-fluorophenyl)amide
[46] 1-oxa-2,8-diazaspiro[4.5]dec-2-ene-3,8-dicarboxylic acid-8-[(3-chlorophenyl)amide]-3-(3,4-dimethoxybenzylamide)
[47] 3-[4-(3-chloropyridin-2-yl)piperazine-1-carbonyl]-1-oxa-2,8-diazaspiro[4.5]dec-2-ene-8-carboxylic acid-(3-chlorophenyl)amide
[48] 1-oxa-2,8-diazaspiro[4.5]dec-2-ene-3,8-dicarboxylic acid-8-[(3-chlorophenyl)amide]-3-[(5-chloropyridin-2-yl)amide]
[49] 1-oxa-2,8-diazaspiro[4.5]dec-2-ene-3,8-dicarboxylic acid-8-[(4-chlorophenyl)amide]-3-(3,4-dimethoxybenzylamide)
[50] 1-oxa-2,8-diazaspiro[4.5]dec-2-ene-3,8-dicarboxylic acid-3-(4-chlorobenzylamide)-8-[(4-chlorophenyl)amide]
[51] 3-[4-(3-chloropyridin-2-yl)piperazine-1-carbonyl]-1-oxa-2,8-diazaspiro[4.5]dec-2-ene-8-carboxylic acid-(4-chlorophenyl)amide
[52] 3-[4-(3-chloropyridin-2-yl)piperazine-1-carbonyl]-1-oxa-2,8-diazaspiro[4.5]dec-2-ene-8-carboxylic acid-(4-methoxyphenyl)amide
[53] 1-oxa-2,8-diazaspiro[4.5]dec-2-ene-3,8-dicarboxylic acid-3-(3,4-dimethoxybenzylamide)-8-[(2-methylsulphanylphenyl)amide]
[54] 3-[4-(3-chloropyridin-2-yl)piperazine-1-carbonyl]-1-oxa-2,8-diazaspiro[4.5]dec-2-ene-8-carboxylic acid-(2-methylsulphanylphenyl)amide
[55] 1-oxa-2,8-diazaspiro[4.5]dec-2-ene-3,8-dicarboxylic acid-3-(3,4-dimethoxybenzylamide)-8-[(3-methylsulphanylphenyl)amide]
[56] 3-[4-(3-chloropyridin-2-yl)piperazine-1-carbonyl]-1-oxa-2,8-diazaspiro[4.5]dec-2-ene-8-carboxylic acid-(3-methylsulphanylphenyl)amide
[57] 1-oxa-2,8-diazaspiro[4.5]dec-2-ene-3,8-dicarboxylic acid-3-(3,4-dimethoxybenzylamide)-8-[(4-methylsulphanylphenyl)amide]
[58] 3-[4-(3-chloropyridin-2-yl)piperazine-1-carbonyl]-1-oxa-2,8-diazaspiro[4.5]dec-2-ene-8-carboxylic acid-(4-methylsulphanylphenyl)amide
[59] 1-oxa-2,8-diazaspiro[4.5]dec-2-ene-3,8-dicarboxylic acid-3-[(5-chloropyridin-2-yl)amide]-8-[(4-methylsulphanylphenyl)amide]
[60] 1-oxa-2,8-diazaspiro[4.5]dec-2-ene-3,8-dicarboxylic acid-3-(3,4-dimethoxybenzylamide)-8-[(2-isopropylphenyl)amide]
[61] 3-[4-(3-chloropyridin-2-yl)piperazine-1-carbonyl]-1-oxa-2,8-diazaspiro[4.5]dec-2-ene-8-carboxylic acid-(2-isopropylphenyl)amide
[62] 1-oxa-2,8-diazaspiro[4.5]dec-2-ene-3,8-dicarboxylic acid-3-[(5-chloropyridin-2-yl)amide]-8-[(2-isopropylphenyl)amide]
[63] 3-[4-(3-chloropyridin-2-yl)piperazine-1-carbonyl]-1-oxa-2,8-diazaspiro[4.5]dec-2-ene-8-carboxylic acid-(4-isopropylphenyl)amide
[64] 1-oxa-2,8-diazaspiro[4.5]dec-2-ene-3,8-dicarboxylic acid-3-(3,4-dimethoxybenzylamide)-8-[(2-trifluoromethylphenyl)amide]
[65] 3-[4-(3-chloropyridin-2-yl)piperazine-1-carbonyl]-1-oxa-2,8-diazaspiro[4.5]dec-2-ene-8-carboxylic acid-(2-trifluoromethylphenyl)amide
[66] 1-oxa-2,8-diazaspiro[4.5]dec-2-ene-3,8-dicarboxylic acid-3-[(5-chloropyridin-2-yl)amide]-8-[(2-trifluoromethylphenyl)amide]
[67] 1-oxa-2,8-diazaspiro[4.5]dec-2-ene-3,8-dicarboxylic acid-3-(3,4-dimethoxybenzylamide)-8-[(3-trifluoromethylphenyl)amide]
[68] 3-[4-(3-chloropyridin-2-yl)piperazine-1-carbonyl]-1-oxa-2,8-diazaspiro[4.5]dec-2-ene-8-carboxylic acid-(3-trifluoromethylphenyl)amide
[69] 1-oxa-2,8-diazaspiro[4.5]dec-2-ene-3,8-dicarboxylic acid-3-[(5-chloropyridin-2-yl)amide]-8-[(3-trifluoromethylphenyl)amide]
[70] 1-oxa-2,8-diazaspiro[4.5]dec-2-ene-3,8-dicarboxylic acid-3-(3,4-dimethoxybenzylamide)-8-[(4-trifluoromethylphenyl)amide]
[71] 3-[4-(3-chloropyridin-2-yl)piperazine-1-carbonyl]-1-oxa-2,8-diazaspiro[4.5]dec-2-ene-8-carboxylic acid-(4-trifluoromethylphenyl)amide
[72] 1-oxa-2,8-diazaspiro[4.5]dec-2-ene-3,8-dicarboxylic acid-8-cyclohexylamide-3-(3,4-dimethoxybenzylamide)
[73] 1-oxa-2,8-diazaspiro[4.5]dec-2-ene-3,8-dicarboxylic acid-3-(4-chlorobenzylamide)-8-cyclohexylamide
[74] 1-oxa-2,8-diazaspiro[4.5]dec-2-ene-3,8-dicarboxylic acid-8-benzylamide-3-(3,4-dimethoxybenzylamide)
[75] 1-oxa-2,8-diazaspiro[4.5]dec-2-ene-3,8-dicarboxylic acid-3-(3,4-dimethoxybenzylamide)-8-o-tolylamide
[76] 1-oxa-2,8-diazaspiro[4.5]dec-2-ene-3,8-dicarboxylic acid-3-(4-chlorobenzylamide)-8-o-tolylamide
[77] 1-oxa-2,8-diazaspiro[4.5]dec-2-ene-3,8-dicarboxylic acid-3-[(5-chloropyridin-2-yl)amide]-8-o-tolylamide
[78] 1-oxa-2,8-diazaspiro[4.5]dec-2-ene-3,8-dicarboxylic acid-3-(3,4-dimethoxybenzylamide)-8-[(2-ethylphenyl)amide]
[79] 3-[4-(3-chloropyridin-2-yl)piperazine-1-carbonyl]-1-oxa-2,8-diazaspiro[4.5]dec-2-ene-8-carboxylic acid-(2-ethylphenyl)amide
[80] 1-oxa-2,8-diazaspiro[4.5]dec-2-ene-3,8-dicarboxylic acid-3-[(5-chloropyridin-2-yl)amide]-8-[(2-ethylphenyl)amide]
[81] 1-oxa-2,8-diazaspiro[4.5]dec-2-ene-3,8-dicarboxylic acid-3-(4-chlorobenzylamide)-8-[(4-ethylphenyl)amide]
[82] 1-oxa-2,8-diazaspiro[4.5]dec-2-ene-3,8-dicarboxylic acid-3-[(5-chloropyridin-2-yl)amide]-8-[(4-ethylphenyl)amide]
[83] 1-oxa-2,8-diazaspiro[4.5]dec-2-ene-3,8-dicarboxylic acid-3-(3,4-dimethoxybenzylamide)-8-[(2-fluorophenyl)amide]
[84] 1-oxa-2,8-diazaspiro[4.5]dec-2-ene-3,8-dicarboxylic acid-3-(3,4-dimethoxybenzylamide)-8-p-tolylamide
[85] 1-oxa-2,8-diazaspiro[4.5]dec-2-ene-3,8-dicarboxylic acid-3-(4-chlorobenzylamide)-8-p-tolylamide
[86] 3-[4-(3-chloropyridin-2-yl)piperazine-1-carbonyl]-1-oxa-2,8-diazaspiro[4.5]dec-2-ene-8-carboxylic acid p-tolylamide
[87] 1-oxa-2,8-diazaspiro[4.5]dec-2-ene-3,8-dicarboxylic acid-3-[(5-chloropyridin-2-yl)amide]-8-p-tolylamide
[88] 1-oxa-2,8-diazaspiro[4.5]dec-2-ene-3,8-dicarboxylic acid-3-(4-chlorobenzylamide)-8-[(3-ethylphenyl)amide]
[89] 3-[4-(3-chloropyridin-2-yl)piperazine-1-carbonyl]-1-oxa-2,8-diazaspiro[4.5]dec-2-ene-8-carboxylic acid-(3-ethylphenyl)amide
[90] 1-oxa-2,8-diazaspiro[4.5]dec-2-ene-3,8-dicarboxylic acid-3-[(5-chloropyridin-2-yl)amide]-8-[(3-ethylphenyl)amide]
[91] 1-oxa-2,8-diazaspiro[4.5]dec-2-ene-3,8-dicarboxylic acid-3-(3,4-dimethoxybenzylamide)-8-[(2-propylphenyl)amide]
[92] 1-oxa-2,8-diazaspiro[4.5]dec-2-ene-3,8-dicarboxylic acid-3-(4-chlorobenzylamide)-8-[(2-propylphenyl)amide]
[93] 1-oxa-2,8-diazaspiro[4.5]dec-2-ene-3,8-dicarboxylic acid-3-(4-chlorobenzylamide)-8-[(2-propyl phenyl)amide]
[94] 3-[4-(3-chloropyridin-2-yl)piperazine-1-carbonyl]-1-oxa-2,8-diazaspiro[4.5]dec-2-ene-8-carboxylic acid-(2-propylphenyl)amide
[95] 1-oxa-2,8-diazaspiro[4.5]dec-2-ene-3,8-dicarboxylic acid-3-[(5-chloropyridin-2-yl)amide]-8-[(2-propylphenyl)amide]
[96] 1-oxa-2,8-diazaspiro[4.5]dec-2-ene-3,8-dicarboxylic acid-8-[(2-bromophenyl)amide]-3-(3,4-dimethoxybenzylamide)
[97] 1-oxa-2,8-diazaspiro[4.5]dec-2-ene-3,8-dicarboxylic acid-8-[(2-bromophenyl)amide]-3-(4-chlorobenzylamide)
[98] 3-[4-(3-chloropyridin-2-yl)piperazine-1-carbonyl]-1-oxa-2,8-diazaspiro[4.5]dec-2-ene-8-carboxylic acid-(2-bromophenyl)amide
[99] 3-[4-(3-chloropyridin-2-yl)piperazine-1-carbonyl]-1-oxa-2,8-diazaspiro[4.5]dec-2-ene-8-carboxylic acid-(4-bromophenyl)amide
[100] 3-[4-(3-chloropyridin-2-yl)piperazine-1-carbonyl]-1-oxa-2,8-diazaspiro[4.5]dec-2-ene-8-carboxylic acid-biphenyl-4-ylamide
[101] 1-oxa-2,8-diazaspiro[4.5]dec-2-ene-3,8-dicarboxylic acid-3-(3,4-dimethoxybenzylamide)-8-[(2-phenoxyphenyl)amide]
[102] 3-[4-(3-chloropyridin-2-yl)piperazine-1-carbonyl]-1-oxa-2,8-diazaspiro[4.5]dec-2-ene-8-carboxylic acid-(2-phenoxyphenyl)amide
[103] 1-oxa-2,8-diazaspiro[4.5]dec-2-ene-3,8-dicarboxylic acid-3-[(5-chloropyridin-2-yl)amide]-8-[(2-phenoxyphenyl)amide]
[104] 1-oxa-2,8-diazaspiro[4.5]dec-2-ene-3,8-dicarboxylic acid-3-(4-chlorobenzylamide)-8-[(2-trifluoromethoxyphenyl)amide]
[105] 3-[4-(3-chloropyridin-2-yl)piperazine-1-carbonyl]-1-oxa-2,8-diazaspiro[4.5]dec-2-ene-8-carboxylic acid-(2-trifluoromethoxyphenyl)amide
[106] 1-oxa-2,8-diazaspiro[4.5]dec-2-ene-3,8-dicarboxylic acid-3-[(5-chloropyridin-2-yl)amide]-8-[(2-trifluoromethoxyphenyl)amide]
[107] 1-oxa-2,8-diazaspiro[4.5]dec-2-ene-3,8-dicarboxylic acid-3-(3,4-dimethoxybenzylamide)-8-(4-methylbenzylamide)
[108] 1-oxa-2,8-diazaspiro[4.5]dec-2-ene-3,8-dicarboxylic acid-3-(4-chlorobenzylamide)-8-(4-methylbenzylamide)
[109] 3-[4-(3-chloropyridin-2-yl)piperazine-1-carbonyl]-1-oxa-2,8-diazaspiro[4.5]dec-2-ene-8-carboxylic acid-4-methylbenzylamide
[110] 1-oxa-2,8-diazaspiro[4.5]dec-2-ene-3,8-dicarboxylic acid-3-[(5-chloropyridin-2-yl)amide]-8-(4-methylbenzylamide)
[111] 1-oxa-2,8-diazaspiro[4.5]dec-2-ene-3,8-dicarboxylic acid-3-(3,4-dimethoxybenzylamide)-8-(4-methoxybenzylamide)
[112] 1-oxa-2,8-diazaspiro[4.5]dec-2-ene-3,8-dicarboxylic acid-3-(4-chlorobenzylamide)-8-(4-methoxybenzylamide)
[113] 3-[4-(3-chloropyridin-2-yl)piperazine-1-carbonyl]-1-oxa-2,8-diazaspiro[4.5]dec-2-ene-8-carboxylic acid-4-methoxybenzylamide
[114] 1-oxa-2,8-diazaspiro[4.5]dec-2-ene-3,8-dicarboxylic acid-8-[(4-tert-butylphenyl)amide]-3-(3,4-dimethoxybenzylamide)
[115] 1-oxa-2,8-diazaspiro[4.5]dec-2-ene-3,8-dicarboxylic acid-8-[(4-tert-butylphenyl)amide]-3-(4-chlorobenzylamide)
[116] 3-[4-(3-chloropyridin-2-yl)piperazine-1-carbonyl]-1-oxa-2,8-diazaspiro[4.5]dec-2-ene-8-carboxylic acid-(4-tert-butylphenyl)amide
[117] 1-oxa-2,8-diazaspiro[4.5]dec-2-ene-3,8-dicarboxylic acid-8-[(4-tert-butylphenyl)amide]-3-[(5-chloropyridin-2-yl)amide]
[118] 8-(2-methoxyphenylthiocarbamoyl)-1-oxa-2,8-diazaspiro[4.5]dec-2-ene-3-carboxylic acid-3,4-dimethoxybenzylamide
[119] 3-[4-(3-chloropyridin-2-yl)piperazine-1-carbonyl]-1-oxa-2,8-diazaspiro[4.5]dec-2-ene-8-thiocarboxylic acid-(2-methoxyphenyl)amide
[120] 8-(cyclohexylmethylthiocarbamoyl)-1-oxa-2,8-diazaspiro[4.5]dec-2-ene-3-carboxylic acid-3,4-dimethoxybenzylamide
[121] 8-(cyclohexylmethylthiocarbambyl)-1-oxa-2,8-diazaspiro[4.5]dec-2-ene-3-carboxylic acid-4-chlorobenzylamide
[122] 3-[4-(3-chloropyridin-2-yl)piperazine-1-carbonyl]-1-oxa-2,8-diazaspiro[4.5]dec-2-ene-8-thiocarboxylic acid cyclohexylmethylamide
[123] 8 -cyclooctylthiocarbamoyl-1-oxa-2,8-diazaspiro[4.5]dec-2-ene-3-carboxylic acid-3,4-dimethoxybenzylamide
[124] 8 -cyclooctylthiocarbamoyl-1-oxa-2,8-diazaspiro[4.5]dec-2-ene-3-carboxylic acid-4-chlorobenzylamide
[125] 8-(3-morpholin-4-yl-propylthiocarbamoyl)-1-oxa-2,8-diazaspiro[4.5]dec-2-ene-3-carboxylic acid-4-chlorobenzylamide
[126] 8-(3-morpholin-4-yl-propylthiocarbamoyl)-1-oxa-2,8-diazaspiro[4.5]dec-2-ene-3-carboxylic acid-(5-chloropyridin-2-yl)amide
[127] 8-p-tolylthiocarbamoyl-1-oxa-2,8-diazaspiro[4.5]dec-2-ene-3-carboxylic acid-4-chlorobenzylamide
[128] 8-phenethylthiocarbamoyl-1-oxa-2,8-diazaspiro[4.5]dec-2-ene-3-carboxylic acid-3,4-dimethoxybenzylamide
[129] 8-phenethylthiocarbamoyl-1-oxa-2,8-diazaspiro[4.5]dec-2-ene-3-carboxylic acid-4-chlorobenzylamide
[130] 8-(3-phenyl-propylthiocarbamoyl)-1-oxa-2,8-diazaspiro[4.5]dec-2-ene-3-carboxylic acid-(5-chloropyridin-2-yl)amide
[131] 8-(2-fluorophenylthiocarbamoyl)-1-oxa-2,8-diazaspiro[4.5]dec-2-ene-3-carboxylic acid-3,4-dimethoxybenzylamide
[132] 8-(4-fluorophenylthiocarbamoyl)-1-oxa-2,8-diazaspiro[4.5]dec-2-ene-3-carboxylic acid-3,4-dimethoxybenzylamide
[133] 8-(4-fluorophenylthiocarbamoyl)-1-oxa-2,8-diazaspiro[4.5]dec-2-ene-3-carboxylic acid-4-chlorobenzylamide
[134] 8-(2-chlorophenylthiocarbamoyl)-1-oxa-2,8-diazaspiro[4.5]dec-2-ene-3-carboxylic acid-3,4-dimethoxybenzylamide
[135] 8-(2-chlorophenylthiocarbamoyl)-1-oxa-2,8-diazaspiro[4.5]dec-2-ene-3-carboxylic acid-4-chlorobenzylamide
[136] 8-(3-trifluoromethyl phenylthiocarbamoyl)-1-oxa-2,8-diazaspiro[4.5]dec-2-ene-3-carboxylic acid-4-chlorobenzylamide
[137] 8-(naphthalen-1-ylthiocarbamoyl)-1-oxa-2,8-diazaspiro[4.5]dec-2-ene-3-carboxylic acid-3,4-dimethoxybenzylamide
[138] 8-(naphthalen-1-ylthiocarbamoyl)-1-oxa-2,8-diazaspiro[4.5]dec-2-ene-3-carboxylic acid-3,4-dimethoxybenzylamide
[139] 8 -cyclopentylthiocarbamoyl-1-oxa-2,8-diazaspiro[4.5]dec-2-ene-3-carboxylic acid-3,4-dimethoxybenzylamide
[140] 3-[4-(3-chloropyridin-2-yl)piperazine-1-carbonyl]-1-oxa-2,8-diazaspiro[4.5]dec-2-ene-8-thiocarboxylic acid cyclopentylamide
[141] 8-(2-morpholin-4-ylethylthiocarbamoyl)-1-oxa-2,8-diazaspiro[4.5]dec-2-ene-3-carboxylic acid-3,4-dimethoxybenzylamide
[142] 8-(2-morpholin-4-ylethylthiocarbamoyl)-1-oxa-2,8-diazaspiro[4.5]dec-2-ene-3-carboxylic acid-4-chlorobenzylamide
[143] 8-(3-chlorophenylthiocarbamoyl)-1-oxa-2,8-diazaspiro[4.5]dec-2-ene-3-carboxylic acid-3,4-dimethoxybenzylamide
[144] 3-[4-(3-chloropyridin-2-yl)piperazine-1-carbonyl]-1-oxa-2,8-diazaspiro[4.5]dec-2-ene-8-thiocarboxylic acid-(4-trifluoromethylphenyl)amide
[145] 8-(2-methylsulphanylphenylthiocarbamoyl)-1-oxa-2,8-diazaspiro[4.5]dec-2-ene-3-carboxylic acid-3,4-dimethoxybenzylamide
[146] 8-(2-methylsulphanylphenylthiocarbamoyl)-1-oxa-2,8-diazaspiro[4.5]dec-2-ene-3-carboxylic acid-4-chlorobenzylamide
[147] 3-[4-(3-chloropyridin-2-yl)piperazine-1-carbonyl]-1-oxa-2,8-diazaspiro[4.5]dec-2-ene-8-thiocarboxylic acid-(2-methylsulphanylphenyl)amide
[148] 8-(4-isopropylphenylthiocarbamoyl)-1-oxa-2,8-diazaspiro[4.5]dec-2-ene-3-carboxylic acid-3,4-dimethoxybenzylamide
[149] 8-(4-isopropylphenylthiocarbamoyl)-1-oxa-2,8-diazaspiro[4.5]dec-2-ene-3-carboxylic acid-4-chlorobenzylamide
[150] 8-(2-iodophenylthiocarbamoyl)-1-oxa-2,8-diazaspiro[4.5]dec-2-ene-3-carboxylic acid-3,4-dimethoxybenzylamide
[151] 8-(2-iodophenylthiocarbamoyl)-1-oxa-2,8-diazaspiro[4.5]dec-2-ene-3-carboxylic acid-4-chlorobenzylamide
[152] 8-(2-trifluoromethylphenylthiocarbamoyl)-1-oxa-2,8-diazaspiro[4.5]dec-2-ene-3-carboxylic acid-3,4-dimethoxybenzylamide
[153] 8-(2-trifluoromethylphenylthiocarbamoyl)-1-oxa-2,8-diazaspiro[4.5]dec-2-ene-3-carboxylic acid-4-chlorobenzylamide
[154] 3-[4-(3-chloropyridin-2-yl)piperazine-1-carbonyl]-1-oxa-2,8-diazaspiro[4.5]dec-2-ene-8-thiocarboxylic acid-(2-trifluoromethylphenyl)amide
[155] 8-[(benzo[1,3]dioxol-5-ylmethyl)thiocarbamoyl]-1-oxa-2,8-diazaspiro[4.5]dec-2-ene-3-carboxylic acid-3,4-dimethoxybenzylamide
[156] 8-[(benzo[1,3]dioxol-5-ylmethyl)thiocarbamoyl]-1-oxa-2,8-diazaspiro[4.5]dec-2-ene-3-carboxylic acid-4-chlorobenzylamide
[157] 3-[4-(3-chloropyridin-2-yl)piperazine-1-carbonyl]-1-oxa-2,8-diazaspiro[4.5]dec-2-ene-8-thiocarboxylic acid-(benzo[1,3]dioxol-5-ylmethyl)amide
[158] 3-[4-(3-chloropyridin-2-yl)piperazine-1-carbonyl]-1-oxa-2,8-diazaspiro[4.5]dec-2-ene-8-carboxylic acid-(3-cyanophenyl)amide
[159] 1-oxa-2,8-diazaspiro[4.5]dec-2-ene-3,8-dicarboxylic acid-3-(3,4-dimethoxybenzylamide)-8-[(2,5-dimethoxyphenyl)amide]
[160] 1-oxa-2,8-diazaspiro[4.5]dec-2-ene-3,8-dicarboxylic acid-3-(4-chlorobenzylamide)-8-[(2,5-dimethoxyphenyl)amide]
[161] 3-[4-(3-chloropyridin-2-yl)piperazine-1-carbonyl]-1-oxa-2,8-diazaspiro[4.5]dec-2-ene-8-carboxylic acid-(2,5-dimethoxyphenyl)amide
[162] 1-oxa-2,8-diazaspiro[4.5]dec-2-ene-3,8-dicarboxylic acid-3-(3,4-dimethoxybenzylamide)-8-[(2,4-dimethyl phenyl)amide]
[163] 1-oxa-2,8-diazaspiro[4.5]dec-2-ene-3,8-dicarboxylic acid-3-(4-chlorobenzylamide)-8-[(2,4-dimethylphenyl)amide]
[164] 3-[4-(3-chloropyridin-2-yl)piperazine-1-carbonyl]-1-oxa-2,8-diazaspiro[4.5]dec-2-ene-8-carboxylic acid-(2,4-dimethylphenyl)amide
[165] 1-oxa-2,8-diazaspiro[4.5]dec-2-ene-3,8-dicarboxylic acid-8-butylamide-3-(3,4-dimethoxybenzylamide)
[166] 1-oxa-2,8-diazaspiro[4.5]dec-2-ene-3,8-dicarboxylic acid-8-butylamide-3-(4-chlorobenzylamide)
[167] 3-[4-(3-chloropyridin-2-yl)piperazine-1-carbonyl]-1-oxa-2,8-diazaspiro[4.5]dec-2-ene-8-carboxylic acid butylamide
[168] 1-oxa-2,8-diazaspiro[4.5]dec-2-ene-3,8-dicarboxylic acid-3-(3,4-dimethoxybenzylamide)-8-pentylamide
[169] 1-oxa-2,8-diazaspiro[4.5]dec-2-ene-3,8-dicarboxylic acid-3-(4-chlorobenzylamide)-8-pentylamide
[170] 3-[4-(3-chloropyridin-2-yl)piperazine-1-carbonyl]-1-oxa-2,8-diazaspiro[4.5]dec-2-ene-8-carboxylic acid pentylamide
[171] 1-oxa-2,8-diazaspiro[4.5]dec-2-ene-3,8-dicarboxylic acid-3-(3,4-dimethoxybenzylamide)-8-[(2-ethoxyphenyl)amide]
[172] 1-oxa-2,8-diazaspiro[4.5]dec-2-ene-3,8-dicarboxylic acid-3-(4-chlorobenzylamide)-8-[(2-ethoxyphenyl)amide]
[173] 1-oxa-2,8-diazaspiro[4.5]dec-2-ene-3,8-dicarboxylic acid-3-(3,4-dimethoxybenzylamide)-8-[(4-ethoxyphenyl)amide]
[174] 1-oxa-2,8-diazaspiro[4.5]dec-2-ene-3,8-dicarboxylic acid-8-[(2,4-difluorophenyl)amide]-3-(3,4-dimethoxybenzylamide)
[175] 1-oxa-2,8-diazaspiro[4.5]dec-2-ene-3,8-dicarboxylic acid-3-(4-chlorobenzylamide)-8-[(2,4-difluorophenyl)amide]
[176] 1-oxa-2,8-diazaspiro[4.5]dec-2-ene-3,8-dicarboxylic acid-8-[(3-chloro-2-methylphenyl)amide]-3-(3,4-dimethoxybenzylamide)
[177] 1-oxa-2,8-diazaspiro[4.5]dec-2-ene-3,8-dicarboxylic acid-3-(4-chlorobenzylamide)-8-[(3-chloro-2-methylphenyl)amide]
[178] 1-oxa-2,8-diazaspiro[4.5]dec-2-ene-3,8-dicarboxylic acid-8-[(4-chloro-2-methylphenyl)amide]-3-(3,4-dimethoxybenzylamide)
[179] 1-oxa-2,8-diazaspiro[4.5]dec-2-ene-3,8-dicarboxylic acid-3-(4-chlorobenzylamide)-8-[(4-chloro-2-methylphenyl)amide]
[180] 1-oxa-2,8-diazaspiro[4.5]dec-2-ene-3,8-dicarboxylic acid-8-[(3-chloro-4-methylphenyl)amide]-3-(3,4-dimethoxybenzylamide)
[181] 1-oxa-2,8-diazaspiro[4.5]dec-2-ene-3,8-dicarboxylic acid-3-(4-chlorobenzylamide)-8-[(3-chloro-4-methylphenyl)amide]
[182] 1-oxa-2,8-diazaspiro[4.5]dec-2-ene-3,8-dicarboxylic acid-8-[(3-chloro-4-fluorophenyl)amide]-3-(3,4-dimethoxybenzylamide)
[183] 1-oxa-2,8-diazaspiro[4.5]dec-2-ene-3,8-dicarboxylic acid-3-(4-chlorobenzylamide)-8-[(3-chloro-4-fluorophenyl)amide]
[184] 1-oxa-2,8-diazaspiro[4.5]dec-2-ene-3,8-dicarboxylic acid-8-[(2,6-diisopropyl phenyl)amide]-3-(3,4-dimethoxybenzylamide)
[185] 1-oxa-2,8-diazaspiro[4.5]dec-2-ene-3,8-dicarboxylic acid-3-(4-chlorobenzylamide)-8-[(2,6-diisopropylphenyl)amide]
[186] 1-oxa-2,8-diazaspiro[4.5]dec-2-ene-3,8-dicarboxylic acid-8-[(5-chloro-2-methoxyphenyl)amide]-3-(3,4-dimethoxybenzylamide)
[187] 1-oxa-2,8-diazaspiro[4.5]dec-2-ene-3,8-dicarboxylic acid-3-(4-chlorobenzylamide)-8-[(5-chloro-2-methoxyphenyl)amide]
[188] 1-oxa-2,8-diazaspiro[4.5]dec-2-ene-3,8-dicarboxylic acid-3-(4-chlorobenzylamide)-8-[(3,4,5-trimethoxyphenyl)amide]
[189] 1-oxa-2,8-diazaspiro[4.5]dec-2-ene-3,8-dicarboxylic acid-3-(3,4-dimethoxybenzylamide)-8-[(3,5-dimethyl phenyl)amide]
[190] 1-oxa-2,8-diazaspiro[4.5]dec-2-ene-3,8-dicarboxylic acid-3-(4-chlorobenzylamide)-8-[(3,5-dimethylphenyl)amide]
[191] 1-oxa-2,8-diazaspiro[4.5]dec-2-ene-3,8-dicarboxylic acid-3-(3,4-dimethoxybenzylamide)-8-[(2,6-dimethylphenyl)amide]
[192] 1-oxa-2,8-diazaspiro[4.5]dec-2-ene-3,8-dicarboxylic acid-3-(4-chlorobenzylamide)-8-[(2,6-dimethylphenyl)amide]
[193] 1-oxa-2,8-diazaspiro[4.5]dec-2-ene-3,8-dicarboxylic acid-3-(3,4-dimethoxybenzylamide)-8-[(3,4-dimethylphenyl)amide]
[194] 1-oxa-2,8-diazaspiro[4.5]dec-2-ene-3,8-dicarboxylic acid-3-(3,4-dimethoxybenzylamide)-8-[(2,5-dimethylphenyl)amide]
[195] 1-oxa-2,8-diazaspiro[4.5]dec-2-ene-3,8-dicarboxylic acid-3-(4-chlorobenzylamide)-8-[(2,5-dimethylphenyl)amide]
[196] 1-oxa-2,8-diazaspiro[4.5]dec-2-ene-3,8-dicarboxylic acid-3-(3,4-dimethoxybenzylamide)-8-[(2-ethyl-6-methylphenyl)amide]
[197] 1-oxa-2,8-diazaspiro[4.5]dec-2-ene-3,8-dicarboxylic acid-3-(4-chlorobenzylamide)-8-[(2-ethyl-6-methylphenyl)amide]
[198] 1-oxa-2,8-diazaspiro[4.5]dec-2-ene-3,8-dicarboxylic acid-3-(3,4-dimethoxybenzylamide)-8-[(4-methoxy-2-methylphenyl)amide]
[199] 1-oxa-2,8-diazaspiro[4.5]dec-2-ene-3,8-dicarboxylic acid-3-(4-chlorobenzylamide)-8-[(4-methoxy-2-methylphenyl)amide]
[200] 1-oxa-2,8-diazaspiro[4.5]dec-2-ene-3,8-dicarboxylic acid-3-(3,4-dimethoxybenzylamide)-8-[(2-methoxy-5-methylphenyl)amide]
[201] 1-oxa-2,8-diazaspiro[4.5]dec-2-ene-3,8-dicarboxylic acid-3-(4-chlorobenzylamide)-8-[(2-methoxy-5-methylphenyl)amide]
[202] 1-oxa-2,8-diazaspiro[4.5]dec-2-ene-3,8-dicarboxylic acid-3-(3,4-dimethoxybenzylamide)-8-[(2,4,5-trimethylphenyl)amide]
[203] 1-oxa-2,8-diazaspiro[4.5]dec-2-ene-3,8-dicarboxylic acid-3-(4-chlorobenzylamide)-8-[(2,4,5-trimethylphenyl)amide]
[204] 1-oxa-2,8-diazaspiro[4.5]dec-2-ene-3,8-dicarboxylic acid-3-(3,4-dimethoxybenzylamide)-8-[(2,4,6-trimethylphenyl)amide]
[205] 1-oxa-2,8-diazaspiro[4.5]dec-2-ene-3,8-dicarboxylic acid-3-(4-chlorobenzylamide)-8-[(2,4,6-trimethylphenyl)amide]
[206] 1-oxa-2,8-diazaspiro[4.5]dec-2-ene-3,8-dicarboxylic acid-8-[(2-bromoethyl)amide]-3-(3,4-dimethoxybenzylamide)
[207] 1-oxa-2,8-diazaspiro[4.5]dec-2-ene-3,8-dicarboxylic acid-8-[(2-bromoethyl)amide]-3-(4-chlorobenzylamide)
[208] 1-oxa-2,8-diazaspiro[4.5]dec-2-ene-3,8-dicarboxylic acid-8-[(4-butyl phenyl)amide]-3-(3,4-dimethoxybenzylamide)
[209] 1-oxa-2,8-diazaspiro[4.5]dec-2-ene-3,8-dicarboxylic acid-8-[(4-butyl phenyl)amide]-3-(4-chlorobenzylamide)
[210] 2-{[3-(4-chlorobenzylcarbamoyl)-1-oxa-2,8-diazaspiro[4.5]dec-2-ene-8-carbonyl]amino}benzoic acid methyl ester
[211] 1-oxa-2,8-diazaspiro[4.5]dec-2-ene-3,8-dicarboxylic acid-8-biphenyl-2-ylamide-3-(3,4-dimethoxybenzylamide)
[212] 1-oxa-2,8-diazaspiro[4.5]dec-2-ene-3,8-dicarboxylic acid-8-biphenyl-2-ylamide-3-(4-chlorobenzylamide)
[213] 1-oxa-2,8-diazaspiro[4.5]dec-2-ene-3,8-dicarboxylic acid-8-[(2,6-dichlorophenyl)amide]-3-(3,4-dimethoxybenzylamide)
[214] 1-oxa-2,8-diazaspiro[4.5]dec-2-ene-3,8-dicarboxylic acid-3-(4-chlorobenzylamide)-8-[(2,6-dichlorophenyl)amide]
[215] 3-{[3-(3,4-dimethoxybenzylcarbamoyl)-1-oxa-2,8-diazaspiro[4.5]dec-2-ene-8-carbonyl]amino}benzoic acid ethyl ester
[216] 3-{[3-(4-chlorobenzylcarbamoyl)-1-oxa-2,8-diazaspiro[4.5]dec-2-ene-8-carbonyl]amino}benzoic acid ethyl ester
[217] 4-{[3-(3,4-dimethoxybenzylcarbamoyl)-1-oxa-2,8-diazaspiro[4.5]dec-2-ene-8-carbonyl]amino}benzoic acid ethyl ester
[218] 4-{[3-(4-chlorobenzylcarbamoyl)-1-oxa-2,8-diazaspiro[4.5]dec-2-ene-8-carbonyl]amino}benzoic acid ethyl ester
[219] 1-oxa-2,8-diazaspiro[4.5]dec-2-ene-3,8-dicarboxylic acid-3-(4-chlorobenzylamide)-8-naphthalen-1-ylamide
[220] 3-[4-(3-chloropyridin-2-yl)piperazine-1-carbonyl]-1-oxa-2,8-diazaspiro[4.5]dec-2-ene-8-thiocarboxylic acid-(2-chloro-5-trifluoromethylphenyl)amide
[221] 8-(4-chloro-3-trifluoromethylphenylthiocarbamoyl)-1-oxa-2,8-diazaspiro[4.5]dec-2-ene-3-carboxylic acid-3,4-dimethoxybenzylamide
[222] 8-(3,5-bis-trifluoromethylphenylthiocarbamoyl)-1-oxa-2,8-diazaspiro[4.5]dec-2-ene-3-carboxylic acid-3,4-dimethoxybenzylamide
[223] 8-(3,5-bis-trifluoromethylphenylthiocarbamoyl)-1-oxa-2,8-diazaspiro[4.5]dec-2-ene-3-carboxylic acid-4-chlorobenzylamide
[224] 3-[4-(3-chloropyridin-2-yl)piperazine-1-carbonyl]-1-oxa-2,8-diazaspiro[4.5]dec-2-ene-8-thiocarboxylic acid-(3,5-bis-trifluoromethylphenyl)amide
[225] 8 -cyclododecylthiocarbamoyl-1-oxa-2,8-diazaspiro[4.5]dec-2-ene-3-carboxylic acid-3,4-dimethoxybenzylamide
[226] 8-benzylthiocarbamoyl-1-oxa-2,8-diazaspiro[4.5]dec-2-ene-3-carboxylic acid-3,4-dimethoxybenzylamide
[227] 8-benzylthiocarbamoyl-1-oxa-2,8-diazaspiro[4.5]dec-2-ene-3-carboxylic acid-4-chlorobenzylamide
[228] 3-[4-(3-trifluoromethylpyridin-2-yl)piperazine-1-carbonyl]-1-oxa-2,8-diazaspiro[4.5]dec-2-ene-8-carboxylic acid-(4-tert-butyl phenyl)amide
[229] 3-[4-(3-chloropyridin-2-yl)piperazine-1-carbonyl]-1-oxa-2,8-diaza-spiro[4.5]dec-2-ene-8-carboxylic acid-(4-pentafluorosulphanylphenyl)amide
[230] N3-((5-methylfuran-2-yl)methyl)-N-8-(4-(trifluoromethyl)phenyl)-1-oxa-2,8-diazaspiro[4.5]dec-2-ene-3,8-dicarboxamide,
[231] N8-(4-methoxyphenyl)-N-3-((5-methylfuran-2-yl)methyl)-1-oxa-2,8-diazaspiro[4.5]dec-2-ene-3,8-dicarboxamide,
[232] N8-(4-chlorophenyl)-N-3-((5-methylfuran-2-yl)methyl)-1-oxa-2,8-diazaspiro[4.5]dec-2-ene-3,8-dicarboxamide,
[233] N3-(4-(3-chloropyridin-2-yl)piperazin-1-yl)-N-8-(3,4-dichlorophenyl)-1-oxa-2,8-diazaspiro[4.5]dec-2-ene-3,8-dicarboxamide,
[234] N3-(4-(3-chloropyridin-2-yl)piperazin-1-yl)-N-8-(4-methoxyphenyl)-1-oxa-2,8-diazaspiro[4.5]dec-2-ene-3,8-dicarboxamide,
[235] N8-(4-chlorobenzyl)-N-3-((5-methylfuran-2-yl)methyl)-1-oxa-2,8-diazaspiro[4.5]dec-2-ene-3,8-dicarboxamide,
[236] N8-(3,4-dichlorobenzyl)-N-3-((5-methylfuran-2-yl)methyl)-1-oxa-2,8-diazaspiro[4.5]dec-2-ene-3,8-dicarboxamide,
[237] N3-(4-chlorobenzyl)-N-8-(3,4-dichlorobenzyl)-1-oxa-2,8-diazaspiro[4.5]dec-2-ene-3,8-dicarboxamide,
[238] N8-(3,4-dichlorobenzyl)-N-3-(4-hydroxy-3-methoxybenzyl)-1-oxa-2,8-diazaspiro[4.5]dec-2-ene-3,8-dicarboxamide,
[239] N3-(4-tert-butylbenzyl)-N-8-(3,4-dichlorobenzyl)-1-oxa-2,8-diazaspiro[4.5]dec-2-ene-3,8-dicarboxamide,
[240] N8-(3,4-dichlorobenzyl)-N-3-(4-(trifluoromethyl)benzyl)-1-oxa-2,8-diazaspiro[4.5]dec-2-ene-3,8-dicarboxamide,
[241] 3-(4-(3-chloropyridin-2-yl)piperazine-1-carbonyl)-N-(3,4-dichlorobenzyl)-1-oxa-2,8-diazaspiro[4.5]dec-2-ene-8-carboxamide,
[242] N3-(4-(3-chloropyridin-2-yl)piperazin-1-yl)-N-8-(3,4-dichlorobenzyl)-1-oxa-2,8-diazaspiro[4.5]dec-2-ene-3,8-dicarboxamide,
[243] N8-(3,4-dichlorophenyl)-N-3-((5-methylfuran-2-yl)methyl)-1-oxa-2,8-diazaspiro[4.5]dec-2-ene-3,8-dicarboxamide,
[244] N8-(4-chlorophenyl)-N-3-(4-hydroxy-3-methoxybenzyl)-1-oxa-2,8-diazaspiro[4.5]dec-2-ene-3,8-dicarboxamide,
[245] N8-(3,4-dichlorophenyl)-N-3-(4-hydroxy-3-methoxybenzyl)-1-oxa-2,8-diazaspiro[4.5]dec-2-ene-3,8-dicarboxamide,
[246] N3-(4-chlorobenzyl)-N-8-(3,4-dichlorophenyl)-1-oxa-2,8-diazaspiro[4.5]dec-2-ene-3,8-dicarboxamide,
[247] N3-(4-chlorobenzyl)-N-8-(4-chlorophenyl)-1-oxa-2,8-diazaspiro[4.5]dec-2-ene-3,8-dicarboxamide,
[248] N3-(4-hydroxy-3-methoxybenzyl)-N-8-(4-(trifluoromethyl)phenyl)-1-oxa-2,8-diazaspiro[4.5]dec-2-ene-3,8-dicarboxamide,
[249] N3-(4-hydroxy-3-methoxybenzyl)-N-8-(4-methoxyphenyl)-1-oxa-2,8-diazaspiro[4.5]dec-2-ene-3,8-dicarboxamide,
[250] N3-(4-chlorobenzyl)-N-8-(4-(trifluoromethyl)phenyl)-1-oxa-2,8-diazaspiro[4.5]dec-2-ene-3,8-dicarboxamide,
[251] N8-(3,4-dichlorobenzyl)-N-3-(3,4-dimethoxybenzyl)-1-oxa-2,8-diazaspiro[4.5]dec-2-ene-3,8-dicarboxamide,
[252] N3-(3,4-dimethoxybenzyl)-N-8-(4-(trifluoromethyl)phenyl)-1-oxa-2,8-diazaspiro[4.5]dec-2-ene-3,8-dicarboxamide,
[253] 3-[4-(3-trifluoromethylpyridin-2-yl)piperazine-1-carbonyl]-1-oxa-2,8-diazaspiro[4.5]dec-2-ene-8-carboxylic acid-(4-pentafluorosulphanylphenyl)amide,
[254] N-(4-tert-butylphenyl)-3-(4-(3-chloropyridin-2-yl)-2-methyl piperazine-1-carbonyl)-1-oxa-2,8-diazaspiro[4.5]dec-2-ene-8-carboxamide and
[255] N-(4-tert-butylbenzyl)-3-(4-(3-chloropyridin-2-yl)piperazine-1-carbonyl)-1-oxa-2,8-diazaspiro[4.5]dec-2-ene-8-carboxamide.

Preference may also be given to substituted spiro compounds according to the invention of general formula I which in a FLIPR assay in a concentration of 10 μM display inhibition of the Ca²⁺ ion inflow in dorsal root ganglia of rats of at least 10%, preferably of at least 30%, particularly preferably of at least 50%, most particularly preferably of at least 70%, even more preferably of at least 90%, compared to the maximum achievable inhibition of the Ca²⁺ ion inflow with capsaicin in a concentration of 10 μM.

In the FLIPR assay, the Ca²⁺ inflow is quantified using a Ca²⁺-sensitive dye (type Fluo-4, Molecular Probes Europe BV, Leiden, Netherlands) in a fluorescent imaging plate reader (FLIPR, Molecular Devices, Sunnyvale, USA), as described hereinafter.

The present invention further relates to a process for preparing compounds according to the invention of the above-indicated general formula I in which at least one compound of general formula II,

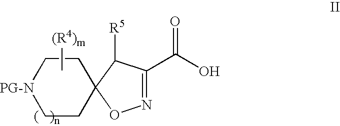

wherein R⁴, R⁵, m and n are as defined above and PG represents a protective group, preferably a benzyloxycarbonyl or tert-butyloxycarbonyl group, is reacted in a reaction medium in the presence of at least one coupling reagent, optionally in the presence of at least one base, with a compound of general formula HNR²R³, wherein R² and R³ are as defined above, to form at least one compound of general formula III,

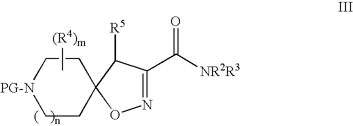

wherein R², R³, R⁴, R⁵, m, n and PG are as defined above, and the at least one compound of general formula III is optionally purified and/or isolated
and
at least one compound of general formula III is reacted in a reaction medium in the presence of at least one acid, preferably in the presence of at least one inorganic or organic acid selected from the group consisting of hydrochloric acid, sulphuric acid, acetic acid and trifluoroacetic acid, or in the presence of hydrogen and a catalyst, preferably a catalyst based on palladium or platinum, to form at least one compound of general formula IV,

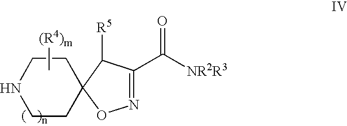

wherein R², R³, R⁴, R⁵, m and n are as defined above, and the at least one compound of general formula IV is optionally purified and/or isolated
and
at least one compound of general formula IV is reacted in a reaction medium with at least one isocyanate of general formula R⁶—N═C═O, wherein R⁶ is as defined above, optionally in the presence of at least one base, preferably in the presence of at least one base selected from the group consisting of triethylamine, 4,4-dimethylaminopyridine and diisopropylethylamine, to form at least one compound of general formula I, wherein R², R³, R⁴, R⁵, m and n are as defined above and R¹ represents —C(═O)—NR⁶R⁷, wherein R⁶ is as defined above and R⁷ represents a hydrogen radical, and the at least one compound of general formula I is optionally purified and/or isolated
or
at least one compound of general formula IV is reacted in a reaction medium with at least one isothiocyanate of general formula S═C═N—R⁸, wherein R⁸ is as defined above, optionally in the presence of at least one base, preferably in the presence of at least one base selected from the group consisting of triethylamine, 4,4-dimethylaminopyridine and diisopropylethylamine, to form at least one compound of general formula I, wherein R², R³, R⁴, R⁵, m and n are as defined above and R¹ represents —C(═S)—N—R⁸R⁹, wherein R⁶ is as defined above and R⁹ represents a hydrogen radical, and the at least one compound of general formula I is optionally purified and/or isolated
and optionally at least one compound of general formula I, wherein R², R³, R⁴, R⁵, m and n are as defined above and R¹ represents —C(═O)—NR⁶R⁷ or —C(═S)—N—R⁸R⁹, wherein R⁷ and R⁹ each represent a hydrogen radical, is reacted in a reaction medium, in the presence of at least one base, preferably in the presence of at least one metal hydride salt or a metal alcoholate salt, particularly preferably in the presence of a metal hydride salt or a metal alcoholate salt selected from the group consisting of sodium hydride, potassium hydride, potassium tert-butanolate, sodium tert-butanolate, potassium methanolate, sodium methanolate, sodium ethanolate and potassium ethanolate, with at least one compound of general formula LG-R⁷ or of general formula LG-R⁹, wherein LG represents a leaving group, preferably a halogen atom, particularly preferably a chlorine atom, and R⁷ and R⁹ are as defined above except for hydrogen, to form at least one compound of general formula I, wherein R¹ to R⁵, m and n are as defined above, and the at least one compound of general formula I is optionally purified and/or isolated,
or
at least one compound of general formula IV is reacted in a reaction medium, in the presence of at least one base, preferably in the presence of at least one metal hydride salt, particularly preferably in the presence of sodium and/or potassium hydride, with at least one compound of general formula LG-R¹, wherein R¹ is as defined above, except for —C(═O)—NR⁶R⁷ and —C(═S)—NR⁸R⁹, and LG represents a leaving group, preferably a halogen atom, particularly preferably a chlorine atom, to form at least one compound of general formula I, wherein R¹ to R⁵, m and n are as defined above, and the at least one compound of general formula I is optionally purified and/or isolated
or
at least one compound of general formula IV is reacted in a reaction medium, in the presence of at least one reducing agent, with at least one compound of general formula R¹—C(═O)—H, wherein R¹ is as defined above, except for —C(═O)—NR⁶R⁷ and —C(═S)—NR⁸R⁹, to form at least one compound of general formula I, wherein R¹ to R⁵, m and n are as defined above, and the at least one compound of general formula I is optionally purified and/or isolated.

The processes according to the invention for preparing substituted spiro compounds of the above-indicated general formula I are also represented in the following Diagrams 1 and 2.

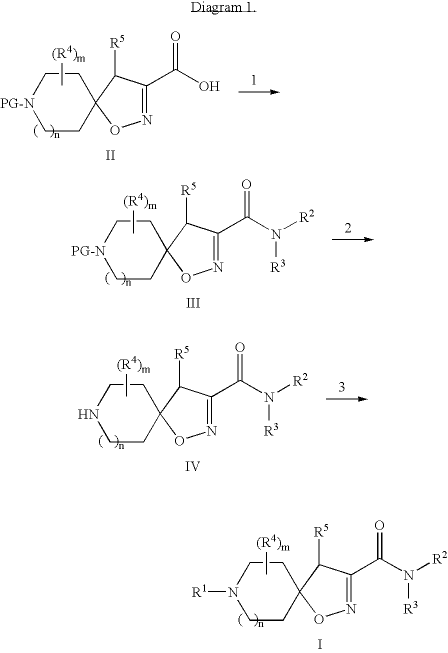

In step 1, compounds of the above-indicated general formula II are reacted with amines of general formula HNR²R³, wherein R¹ and R² are as defined above, in a reaction medium, preferably selected from the group consisting of diethyl ether, tetrahydrofuran, acetonitrile, methanol, ethanol, dimethylformamide, dichloromethane and corresponding mixtures, optionally in the presence of at least one coupling reagent, preferably selected from the group consisting of 1-benzotriazolyloxy-tris-(dimethylamino)phosphonium hexafluorophosphate (BOP), dicyclohexylcarbodiimide (DCC), N′-(3-dimethylaminopropyl)-N-ethylcarbodiimide (EDCl), N-[(dimethyamino)-1H-1,2,3-triazolo[4,5-b]pyridino-1-ylmethylene]-N-methylmethanaminium hexafluorophosphate N-oxide (HATU), O-(benzotriazol-1-yl)-N,N,N′,N′-tetramethyl uronium hexafluorophosphate (H BTU), O-(benzotriazol-1-yl)-N,N,N′,N′-tetramethyluronium tetrafluoroborate (TBTU), 1-hydroxybenzotriazole (HOBt) and 1-hydroxy-7-azabenzotriazole (HOAt), optionally in the presence of at least one inorganic base, preferably selected from the group consisting of potassium carbonate and caesium carbonate, or an organic base, preferably selected from the group consisting of triethylamine, N-methylmorpholine, pyridine, N,N-dimethylaminopyridine and diisopropylethylamine, preferably at temperatures of −70° C. to 100° C. to form compounds of general formula I.

In step 2, compounds of general formula III, wherein PG represents a tert-butyloxycarbonyl group, are reacted in a reaction medium preferably selected from the group consisting of methanol, ethanol, isopropanol, water, diethyl ether, tetrahydrofuran and corresponding mixtures in the presence of at least one acid preferably selected from the group consisting of hydrochloric acid, sulphuric acid, trifluoroacetic acid and acetic acid at temperatures of preferably 20 to 30° C. to form compounds of general formula IV. Particularly preferably, the compound of general formula III is reacted in a 5 M hydrochloric acid solution in isopropanol at a temperature of preferably 20 to 30° C. to form a compound of general formula IV in the form of a corresponding hydrochloride.

Alternatively, compounds of general formula III, wherein PG represents a benzyloxycarbonyl group, are reacted in a reaction medium preferably selected from the group consisting of diethyl ether, tetrahydrofuran, dioxane, acetonitrile, toluene and corresponding mixtures in the presence of hydrogen and palladium on carbon at a temperature of preferably 20 to 80° C. to form a compound of general formula IV.

If compounds of general formula IV are present in the form of a corresponding hydrochloride, they are reacted in a reaction medium, preferably selected from the group consisting of dioxane, tetrahydrofuran, diethyl ether, methanol, ethanol, isopropanol, water and corresponding mixtures, in the presence of an inorganic base, preferably with the addition of a metal hydroxide, for example sodium hydroxide, potassium hydroxide or lithium hydroxide, at temperatures of preferably 0° C. to 30° C., to form the corresponding bases of general formula IV.

In step 3, compounds of general formula IV are reacted with an isocyanate of general formula R⁶—N═C═O, wherein R⁶ is as defined above, or with an isothiocyanate of general formula R⁸—N═C═S, wherein R⁸ is as defined above, in a reaction medium, preferably selected from the group consisting of acetonitrile, toluene, dimethylformamide, benzene, ethanol, methanol, water and corresponding mixtures, optionally in the presence of at least one base, preferably in the presence of at least one base selected from the group consisting of triethylamine, N-methylmorpholine, pyridine, 4,4-dimethylaminopyridine and diisopropylethylamine, to form compounds of general formula I, wherein R¹ represents —C(═O)—NR⁶R⁷ or —C(═S)—NR⁸R⁹, and R⁷ and R⁹ each represent a hydrogen radical.

Also in step 3, compounds of general formula IV are reacted with compounds of general formula LG-R¹, wherein R¹ is as defined above and LG represents a leaving group, preferably a halogen atom, particularly preferably a chlorine atom, in a reaction medium, preferably selected from the group consisting of dichloromethane, toluene, tetrahydrofuran, acetonitrile, diethyl ether, dioxane and corresponding mixtures, optionally in the presence of at least one base, preferably in the presence of at least one metal hydride salt, particularly preferably in the presence of sodium and/or potassium hydride, to form compounds of general formula I, wherein R¹ is as defined above except for —C(═O)—NR⁶R⁷ and —C(═S)—NR⁸R⁹.

Alternatively in step 1, compounds of general formula IV are reacted with compounds of general formula R¹—C(═O)—H, wherein R¹ is as defined above, in a reaction medium, preferably selected from the group consisting of diethyl ether, tetrahydrofuran, methanol, ethanol, dichloromethane, toluene and corresponding mixtures, with the addition of at least one reducing agent, preferably with the addition of at least one reducing agent selected from the group consisting of sodium borohydride, sodium acetoxyborohydride, sodium cyanoborohydride and borane-pyridine complex (pyridine borane, BH₃.C₅H₅N), particularly preferably in the presence of borane-pyridine complex, to form compounds of general formula I, wherein R¹ is as defined above except for —C(═O)—NR⁶R⁷ and —C(═S)—NR⁸R⁹.

The compounds of general formula II may be obtained as illustrated in Diagram 2.

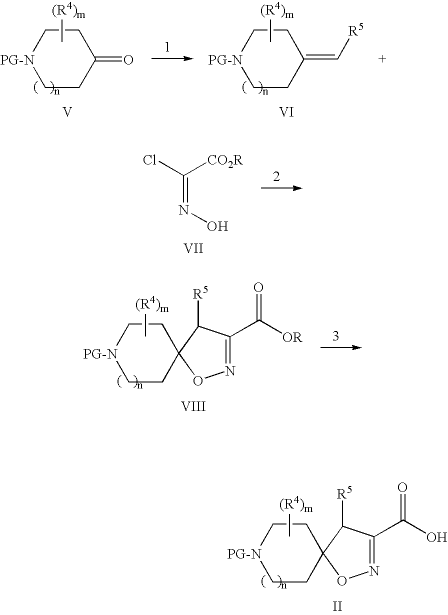

In step 1, compounds of general formula V, wherein m, n, R⁴ and PG are as defined above, are reacted in a reaction medium, preferably in a reaction medium selected from the group consisting of tetrahydrofuran, toluene, diethyl ether and corresponding mixtures, with a reagent for converting carbonyl groups into double bonds, preferably with a Wittig reagent of general formula R₃P(CH₂)R⁵X; wherein R represents an aryl radical, X represents a halogen atom and R⁵ is as defined above; or a Wittig-Horner reagent of general formula (RO)₂—P(═O)—(CH₂)—R⁵, wherein R represents an aryl radical and R⁵ is as defined above, particularly preferably with methyltriphenylphosphonium bromide, at temperatures between 0° C. and 30° C. in the presence of a base, preferably in the presence of an alkali metal alcoholate salt, particularly preferably in the presence of potassium tert-butylate, to form compounds of general formula VI, wherein m, n, R⁴, R⁵ and PG are as defined above.

In step 2, compounds of general formula VI are reacted in a reaction medium, preferably in a reaction medium selected from the group consisting of methanol, tetrahydrofuran, dichloromethane and corresponding mixtures, in the presence of at least one base, preferably in the presence of sodium hydrogen carbonate, lithium hydroxide, triethylamine or N-diisopropylethylamine, with compounds of general formula VII, wherein R represents a linear or branched C₁₋₆ alkyl radical, particularly preferably with compounds of general formula VII, wherein R represents an ethyl radical, i.e. with ethyl chloroximidoacetate, at temperatures between 0° C. and 100° C. to form compounds of general formula VIII, wherein m, n, PG, R⁴, R⁵ and R are as defined above.

In step 3, compounds of general formula VIII are reacted in a reaction medium, preferably in a reaction medium selected from the group consisting of methanol, ethanol, water, isopropanol and corresponding mixtures, in the presence of at least one base, preferably in the presence of lithium hydroxide monohydrate, at temperatures between 0° C. and 50° C. to form compounds of general formula II.

The compounds of the above-indicated formulae R₃P(CH₂)R⁵X, (RO)₂—P(═O)—(CH₂)—R⁵, R¹—C(═O)—H, LG-R¹, LG-R⁷, LG-R⁹, HNR²R³, R⁶—N═C═O, R⁸—N═C═S, V and VII are each commercially available and can also be prepared using conventional processes known to a person skilled in the art.

The above-described reactions can each be carried out under the conventional conditions with which a person skilled in the art is familiar, for example with regard to pressure or the order in which the components are added. If appropriate, a person skilled in the art can determine by simple preliminary tests the procedure which is optimal under the respective conditions. The intermediate and end products obtained as a result of the above-described reactions can each, if it is desirable and/or necessary, be purified and/or isolated using conventional methods known to a person skilled in the art. Suitable purification processes include, for example, extraction processes and chromatographic processes such as column chromatography or preparative chromatography. All of the above-described process steps and in each case also the purification and/or isolation of intermediate or end products can be carried out partly or completely under an inert gas atmosphere, preferably under a nitrogen atmosphere.

The substituted spiro compounds according to the invention of the above-mentioned general formula I, Ic and Id, referred to hereinafter simply as spiro compounds of general formula I, and corresponding stereoisomers can be isolated both in the form of their free bases, their free acids and also in the form of corresponding salts, in particular physiologically compatible salts.

The free bases of the respective substituted spiro compounds according to the invention of the above-mentioned general formula I and of corresponding stereoisomers can, for example, be converted by reaction with an inorganic or organic acid, preferably with hydrochloric acid, hydrobromic acid, sulphuric acid, phosphoric acid, methanesulphonic acid, p-toluenesulphonic acid, carbonic acid, formic acid, acetic acid, oxalic acid, succinic acid, tartaric acid, mandelic acid, fumaric acid, lactic acid, citric acid, glutamic acid or aspartic acid, into the corresponding salts, preferably physiologically compatible salts. The free bases of the respective substituted spiro compounds of the above-mentioned general formula I and of corresponding stereoisomers can also be converted with the free acid or a salt of a sugar substitute, such as, for example, saccharin, cyclamate or acesulfame, into the corresponding physiologically compatible salts.

Accordingly, the free acids of the substituted spiro compounds of the above-mentioned general formula I and of corresponding stereoisomers can be converted by reaction with a suitable base into the corresponding physiologically compatible salts. Examples include the alkali metal salts, alkaline-earth metal salts or ammonium salts [NHₓR₄₋ₓ]⁺, wherein x=0, 1, 2, 3 or 4 and R represents a linear or branched C₁₋₄ alkyl radical.

The substituted spiro compounds according to the invention of the above-mentioned general formula I and of corresponding stereoisomers can optionally also be obtained, like the corresponding acids, the corresponding bases or salts of these compounds, using conventional methods known to a person skilled in the art in the form of their solvates, preferably in the form of their hydrates.

If the substituted spiro compounds according to the invention of the above-mentioned general formula I are obtained after preparation thereof in the form of a mixture of their stereoisomers, preferably in the form of their racemates or other mixtures of their various enantiomers and/or diastereomers, they can be separated and optionally isolated using conventional methods known to a person skilled in the art. Examples include chromatographic separation processes, in particular liquid chromatography processes under normal pressure or under elevated pressure, preferably MPLC and HPLC processes, and fractionated crystallisation processes.

These allow, in particular, the separation from one another of individual enantiomers of diastereomeric salts formed, for example, by means of HPLC on a chiral stationary phase or by means of crystallisation with chiral acids, for example (+)-tartaric acid, (−)-tartaric acid or (+)-10-camphorsulphonic acid.

The substituted spiro compounds according to the invention of the above-mentioned general formula I and corresponding stereoisomers and in each case the corresponding acids, bases, salts and solvates are toxicologically safe and are therefore suitable as pharmaceutical active ingredients in medicaments.

The present invention therefore further relates to a medicament comprising at least one spiro compound according to the invention of the above-indicated general formula I, in each case optionally in the form of one of its pure stereoisomers, in particular enantiomers or diastereomers, its racemates or in the form of a mixture of stereoisomers, in particular of enantiomers and/or diastereomers, in any desired mixing ratio, or respectively in the form of a corresponding salt, or respectively in the form of a corresponding solvate, and optionally one or more pharmaceutically compatible adjuvants.

These medicaments according to the invention are suitable, in particular, for vanilloid receptor 1 (VR1/TRPV1) regulation, in particular for vanilloid receptor 1 (VR1/TRPV1) inhibition.

Also preferably, the medicaments according to the invention are suitable for the prophylaxis and/or treatment of disturbances or diseases transmitted, at least in some cases, by vanilloid receptors 1.

Preferably, the medicament according to the invention is suitable for the treatment and/or prophylaxis of one or more diseases selected from the group consisting of pain, preferably of pain selected from the group consisting of acute pain, chronic pain, neuropathic pain and visceral pain; arthralgia; migraine; depression; neuropathy; nerve injuries; neurodegenerative diseases, preferably selected from the group consisting of multiple sclerosis, Alzheimer's disease, Parkinson's disease and Huntington's disease; cognitive dysfunctions, preferably cognitive deficiencies, particularly preferably paramnesia; epilepsy; respiratory diseases, preferably selected from the group consisting of asthma and pneumonia; coughing; urinary incontinence; OAB (overactive bladder); stomach ulcers; irritable bowel syndrome; strokes; irritations of the eyes; irritations of the skin; neurotic skin diseases; inflammatory diseases, preferably intestinal inflammations; diarrhoea; pruritus; eating disorders, preferably selected from the group consisting of bulimia, cachexia, anorexia and obesity; medication dependency; medication abuse; withdrawal symptoms in medication dependency; development of tolerance to medication, preferably to natural or synthetic opioids; drug addiction; drug abuse; withdrawal symptoms in drug addiction; alcohol addiction; alcohol abuse and withdrawal symptoms in alcohol addiction; for diuresis; for antinatriuresis; for influencing the cardiovascular system; for increasing vigilance; for increasing libido; for modulating motor activity; for anxiolysis; for local anaesthetics and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Particularly preferably, the medicament according to the invention is suitable for the treatment and/or prophylaxis of one or more diseases selected from the group consisting of pain, preferably of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiencies, particularly preferably paramnesia; urinary incontinence; OAB (overactive bladder); medication dependency; medication abuse; withdrawal symptoms in medication dependency; development of tolerance to medication, preferably development of tolerance to natural or synthetic opioids; drug addiction; drug abuse; withdrawal symptoms in drug addiction; alcohol addiction; alcohol abuse and withdrawal symptoms in alcohol addiction.

Most particularly preferably, the medicament according to the invention is suitable for the treatment and/or prophylaxis of pain, preferably of pain selected from the group consisting of acute pain, chronic pain, neuropathic pain and visceral pain, and/or urinary incontinence.

The present invention further relates to the use of at least one substituted spiro compound according to the invention and optionally of one or more pharmaceutically compatible adjuvants for producing a medicament for vanilloid receptor 1 (VR1/TRPV1) regulation, preferably for vanilloid receptor 1 (VR1/TRPV1) inhibition and/or for vanilloid receptor 1 (VR1/TRPV1) stimulation.

Preferred is the use of at least one substituted spiro compound according to the invention and optionally of one or more pharmaceutically compatible adjuvants for producing a medicament for the prophylaxis and/or treatment of disturbances or diseases which are transmitted, at least in some cases, by vanilloid receptors 1.

Particularly preferred is the use of at least one substituted spiro compound according to the invention and optionally of one or more pharmaceutically compatible adjuvants for producing a medicament for the treatment and/or prophylaxis of one or more diseases selected from the group consisting of pain, preferably of pain selected from the group consisting of acute pain, chronic pain, neuropathic pain and visceral pain; arthralgia; migraine; depression; neuropathy; nerve injuries; neurodegenerative diseases, preferably selected from the group consisting of multiple sclerosis, Alzheimer's disease, Parkinson's disease and Huntington's disease; cognitive dysfunctions, preferably cognitive deficiencies, particularly preferably paramnesia; epilepsy; respiratory diseases, preferably selected from the group consisting of asthma and pneumonia; coughing; urinary incontinence; OAB (overactive bladder); stomach ulcers; irritable bowel syndrome; strokes; irritations of the eyes; irritations of the skin; neurotic skin diseases; inflammatory diseases, preferably intestinal inflammations; diarrhoea; pruritus; eating disorders, preferably selected from the group consisting of bulimia, cachexia, anorexia and obesity; medication dependency; medication abuse; withdrawal symptoms in medication dependency; development of tolerance to medication, preferably to natural or synthetic opioids; drug addiction; drug abuse; withdrawal symptoms in drug addiction; alcohol addiction; alcohol abuse and withdrawal symptoms in alcohol addiction; for diuresis; for antinatriuresis; for influencing the cardiovascular system; for increasing vigilance; for increasing libido; for modulating motor activity; for anxiolysis; for local anaesthetics and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particularly preferred is the use of at least one substituted spiro compound according to the invention and optionally of one or more pharmaceutically compatible adjuvants for producing a medicament for the treatment and/or prophylaxis of one or more diseases selected from the group consisting of pain, preferably of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiencies, particularly preferably paramnesia; urinary incontinence; OAB (overactive bladder); medication dependency; medication abuse; withdrawal symptoms in medication dependency; development of tolerance to medication, preferably development of tolerance to natural or synthetic opioids; drug addiction; drug abuse; withdrawal symptoms in drug addiction; alcohol addiction; alcohol abuse and withdrawal symptoms in alcohol addiction.

Still more preferred is the use of at least one substituted spiro compound according to the invention and optionally of one or more pharmaceutically compatible adjuvants for producing a medicament for the treatment and/or prophylaxis of pain, preferably selected from the group consisting of acute pain, chronic pain, neuropathic pain and visceral pain, and/or urinary incontinence.

The present invention further relates to the use of at least one substituted spiro compound according to the invention, wherein R¹ represents a —C(═O)NR⁶R⁷ group and die remaining radicals are each as defined above, on the condition that m is equal to 0 and n equal to 1 and R⁵ equal to H, and optionally of one or more pharmaceutically compatible adjuvants for producing a medicament for vanilloid receptor 1 (VR1/TRPV1) regulation, preferably for vanilloid receptor 1 (VR1/TRPV1) inhibition and/or for vanilloid receptor 1 (VR1/TRPV1) stimulation.

Preferred is the corresponding use for producing a medicament for the prophylaxis and/or treatment of disturbances or diseases which are transmitted, at least in some cases, by vanilloid receptors 1 (VR1/TRPV1).

Particularly preferred is the corresponding use for producing a medicament for the treatment and/or prophylaxis of one or more diseases selected from the group consisting of acute pain; visceral pain; arthralgia; cognitive dysfunctions, preferably cognitive deficiencies, particularly preferably paramnesia; pneumonia; coughing; OAB (overactive bladder); irritable bowel syndrome (irritable bowel syndrome); irritations of the eyes; irritations of the skin; neurotic skin diseases; intestinal inflammations; development of tolerance to medication, preferably to natural or synthetic opioids; and/or for diuresis or for antinatriuresis and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

The medicament according to the invention is suitable for administration to adults and children, including toddlers and babies. The medicament according to the invention can be provided as a liquid, semisolid or solid pharmaceutical dosage form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, optionally compressed to form tablets, introduced into capsules or suspended in a liquid, and also be administered as such.

In addition to at least one substituted spiro compound of the above-indicated general formula I, optionally in the form of one of its pure stereoisomers, in particular enantiomers or diastereomers, its racemate or in the form of mixtures of stereoisomers, in particular of enantiomers or diastereomers, in any desired mixing ratio, or optionally in the form of a corresponding salt or respectively in the form of a corresponding solvate, the medicament according to the invention conventionally comprises further physiologically compatible pharmaceutical adjuvants which can be selected, for example, from the group consisting of excipients, fillers, solvents, diluting agents, surface-active substances, dyes, preservatives, disintegrants, slip additives, lubricants, aroma substances and binding agents.

The selection of the physiologically compatible adjuvants and the amounts thereof to be used are dependent on whether the medicament is to be administered orally, subcutaneously, parenterally, intravenously, intraperitoneally, intradermally, intramuscularly, intranasally, buccally, rectally or locally, for example to infections on the skin, the mucous membranes and on the eyes. Preferably suitable for oral administration are preparations in the form of tablets, dragées, capsules, granules, pellets, drops, juices and syrups, for parenteral, solutions to be administered topically and by inhalation, suspensions, easily reconstitutable dry preparations and sprays. The substituted spiro compounds according to the invention used in the medicament according to the invention in a repository in dissolved form or in a plaster, optionally with the addition of means promoting skin penetration, are suitable percutaneous administration preparations. Preparation forms to be administered orally or percutaneously can also release the respective substituted spiro compound according to the invention in a delayed manner.

The medicaments according to the invention are prepared using conventional means, devices, methods and processes known in the art such as are described, for example, in “Remington's Pharmaceutical Sciences”, A. R. Gennaro (Editor), 17th edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted spiro compounds according to the invention of the above-indicated general formula I may vary and is, for example, dependent on the patient's weight or age and on the type of administration, the indication and the severity of the disease. Conventionally, 0.001 to 100 mg/kg, preferably 0.05 to 75 mg/kg, particularly preferably 0.05 to 50 mg/kg of the patient's body weight of at least one compound of this type according to the invention are administered.

### Pharmacological Methods:

### I. Functional Testing Carried out on the Vanilloid Receptor 1 (VRI/TRPV1 Receptor)

The agonistic or antagonistic effect of the substances to be tested can be determined on the rat-species vanilloid receptor 1 (VR1/TRPV1) using the following assay. According to this assay, the Ca²⁺ inflow is quantified through the receptor channel using a Ca²⁺-sensitive dye (type Fluo-4, Molecular Probes Europe BV, Leiden, Netherlands) in a fluorescent imaging plate reader (FLIPR, Molecular Devices, Sunnyvale, USA).

### Method:

Complete medium: 50 mL HAMS F12 nutrient mixture (Gibco Invitrogen GmbH, Karlsruhe, Germany) with

10% by volume of FCS (foetal calf serum, Gibco Invitrogen GmbH, Karlsruhe, Germany, heat-activated);

2 mM L-glutamine (Sigma, Munich, Germany);

1% by weight of AA solution (antibiotic/antimyotic solution, PAA, Pasching, Austria) and 25 ng/ml NGF medium (2.5 S, Gibco Invitrogen GmbH, Karlsruhe, Germany)

Cell culture plate: Poly-D-lysine-coated, black 96 well plates having a clear base (96 well black/clear plate, BD Biosciences, Heidelberg, Germany) were additionally coated with laminin (Gibco Invitrogen GmbH, Karlsruhe, Germany), the laminin being diluted with PBS (Ca—Mg-free PBS, Gibco Invitrogen GmbH, Karlsruhe, Germany) to a concentration of 100 μg/mL. Aliquots having a laminin concentration of 100 μg/mL were removed and stored at −20° C. The aliquots were diluted with PBS in a ratio of 1:10 to 10 μg/mL laminin and in each case 50 μL of the solution were pipetted into a recess in the cell culture plate. The cell culture plates were incubated for at least two hours at 37° C., the excess solution was removed by suction-filtration and the recesses were each washed twice with PBS. The coated cell culture plates were stored with excess PBS which was not removed until just before the feeding of the cells.

### Preparation of the Cells:

The vertebral column was removed from decapitated rats and placed immediately into cold HBSS buffer (Hank's buffered saline solution, Gibco Invitrogen GmbH, Karlsruhe, Germany), i.e. buffer located in an ice bath, mixed with 1% by volume (percent by volume) of an AA solution (antibiotic/antimyotic solution, PAA, Pasching, Austria). The vertebral column was cut longitudinally and removed together with fasciae from the vertebral canal. Subsequently, the dorsal root ganglia (DRG) were removed and again stored in cold HBSS buffer mixed with 1% by volume of an AA solution. The DRG, from which all blood remnants and spinal nerves had been removed, were transferred in each case to 500 μL of cold type 2 collagenase (PAA, Pasching, Austria) and incubated for 35 minutes at 37° C. After the addition of 2.5% by volume of trypsin (PAA, Pasching, Austria), incubation was continued for 10 minutes at 37° C. After complete incubation, the enzyme solution was carefully pipetted off and 500 μL of complete medium were added to each of the remaining DRG. The DRG were in each case suspended several times, drawn through cannulae No. 1, No. 12 and No. 16 using a syringe and transferred to a 50 mL Falcon tube which was filled up to 15 mL with complete medium. The contents of each Falcon tube was in each case filtered through a 70 μm Falcon filter element and centrifuged for 10 minutes at 1,200 rpm and room temperature. The resulting pellet was in each case taken up in 250 μL of complete medium and the number of cells determined.

The number of cells in the suspension was set to 3×10⁵ per mL and 150 μL of this suspension were in each case introduced into a recess in the cell culture plates coated as described hereinbefore. In the incubator the plates were left for two to three days at 37° C., 5% by volume of CO₂ and 95% relative humidity.

Subsequently, the cells were loaded with 2 μM of Fluo-4 and 0.01% by volume of Pluronic F127 (Molecular Probes Europe BV, Leiden, Netherlands) in HBSS buffer (Hank's buffered saline solution, Gibco Invitrogen GmbH, Karlsruhe, Germany) for 30 min at 37° C., washed 3 times with HBSS buffer and after further incubation for 15 minutes at room temperature used for Ca²⁺ measurement in a FLIPR assay. The Ca²⁺-dependent fluorescence was measured before and after the addition of substances (λex=488 nm, λem=540 nm). Quantification was carried out by measuring the highest fluorescence intensity (FC, Fluorescence Counts) over time.

### FLIPR Assay:

The FLI PR protocol consists of 2 substance additions. First the compounds to be tested (10 μM) were pipetted onto the cells and the Ca²⁺ inflow was compared with the control (capsaicin 10 μM). This provides the result in % activation based on the Ca²⁺ signal after the addition of 10 μM capsaicin (CP). After 5 minutes' incubation, 100 nM of capsaicin were administered and the Ca²⁺ inflow was also determined.

Desensitising agonists and antagonists led to suppression of the Ca²⁺ inflow. The % inhibition was calculated compared to the maximum achievable inhibition with 10 μM of capsaicin.

Triple analyses (n=3) were carried out and repeated in at least 3 independent experiments (N=4).

### II. Functional Testing Carried out on the Vanilloid Receptor (VR1)

The agonistic or antagonistic effect of the substances to be tested can also be determined on the vanilloid receptor (VR1) using the following assay. According to this assay, the Ca²⁺ inflow is quantified through the channel using a Ca²⁺-sensitive dye (type Fluo-4, Molecular Probes Europe BV, Leiden, Netherlands) in a fluorescent imaging plate reader (FLIPR, Molecular Devices, Sunnyvale, USA).

### Method:

Chinese hamster ovary cells (CHO K1 cells, European Collection of Cell Cultures (ECACC) United Kingdom) were stably transfected with the VR1 gene. For functional testing, these cells were plated out on poly-D-lysine-coated black 96 well plates having a clear base (BD Biosciences, Heidelberg, Germany) at a density of 25,000 cells/well. The cells were incubated overnight at 37° C. and 5% CO₂ in a culture medium (Ham's F12 nutrient mixture, 10-% by volume of FCS (foetal calf serum), 18 μg/ml L-proline). The next day the cells were incubated with Fluo-4 (Fluo-4 2 μM, 0.01% by volume of Pluronic F127, Molecular Probes in HBSS (Hank's buffered saline solution), Gibco Invitrogen GmbH, Karlsruhe, Germany) for 30 minutes at 37° C. Subsequently, the plates were washed 3 times with HBSS buffer and after further incubation for 15 minutes at room temperature used for Ca²⁺ measurement in a FLI PR assay. The Ca²⁺-dependent fluorescence was measured before and after the addition of the substances to be tested (λex wavelength=488 nm, λem=540 nm). Quantification was carried out by measuring the highest fluorescence intensity (FC, Fluorescence Counts) over time.

### FLIPR Assay:

The FLIPR protocol consists of 2 substance additions. First the compounds to be tested (10 μM) were pipetted onto the cells and the Ca²⁺ inflow was compared with the control (capsaicin 10 μM) (% activation based on the Ca²⁺ signal after the addition of 10 μM capsaicin). After 5 minutes' incubation, 100 nM of capsaicin were administered and the Ca²⁺ inflow was also determined.

Desensitising agonists and antagonists led to suppression of the Ca²⁺ inflow. The % inhibition was calculated compared to the maximum achievable inhibition with 10 μM of capsaicin.

### III. Formalin Test Carried Out on Mice

In the formalin test, the testing to determine the antinociceptive effect of the compounds according to the invention was carried out on male mice (NMRI, 20 to 30 g body weight, Iffa, Credo, Belgium).

In the formalin test as described by D. Dubuisson et al., Pain 1977, 4, 161-174, a distinction is drawn between the first (early) phase (0 to 15 minutes after the injection of formalin) and the second (late) phase (15 to 60 minutes after the injection of formalin). The early phase, as an immediate reaction to the injection of formalin, is a model of acute pain, whereas the late phase is regarded as a model of persistent (chronic) pain (T. J. Coderre et al., Pain 1993, 52, 259-285). The corresponding descriptions in the literature are introduced herewith by way of reference and form part of the disclosure.

The compounds according to the invention were tested in the second phase of the formalin test to obtain information about the effects of substances on chronic/inflammatory pain.

The moment at which the compounds according to the invention were administered before the injection of formalin was selected as a function of the type of administration. 10 mg of the test substances/kg of body weight were administered intravenously 5 minutes before the injection of formalin which was carried out by a single subcutaneous injection of formalin (20 μL, 1% aqueous solution) into the dorsal side of the right hind paw, thus inducing in free moving test animals a nociceptive reaction which manifests itself in marked licking and biting of the respective paw.

Subsequently, the nociceptive behaviour was continuously detected by observing the animals over a test period of three minutes in the second (late) phase of the formalin test (21 to 24 minutes after the injection of formalin). The pain behaviour was quantified by adding up the seconds over which the animals displayed licking and biting of the respective paw during the test period.

The comparison was carried out in each case with control animals which were given vehicles (0.9% aqueous sodium chloride solution) instead of the compounds according to the invention before the administration of formalin. Based on the quantification of the pain behaviour, the effect of the substance was determined in the formalin test as a percentage change relative to the corresponding control.

After the injection of substances having an antinociceptive effect in the formalin test, the described behaviour of the animals, i.e. licking and biting, was reduced or eliminated.

The invention will be described hereinafter with reference to a few examples. This description is intended merely by way of example and does not limit the general idea of the invention.

### EXAMPLES

The yields of the compounds prepared are not optimised.

All temperatures are uncorrected.

### Abbreviations:

abs. absolute
aq. aqueous
eq. equivalent amount of substance
Boc tert-butoxycarbonyl
BOP 1-benzotriazolyloxy-tris-(dimethylamino)phosphonium hexafluorophosphate
DCM dichloromethane
DMF dimethylformamide
EtOAc ethyl acetate
sat. saturated
MeOH methanol
NMR nuclear magentic resonance spectroscopy
RT room temperature

The chemicals and solvents used were purchased from the conventional suppliers (Acros, Avocado, Aldrich, Bachem, Fluka, Lancaster, Maybridge, Merck, Sigma, TCI, etc.) or synthesised using the methods known to a person skilled in the art.

The stationary phase used for the column chromatography was silica gel 60 (0.040-0.063 mm) from E. Merck, Darmstadt.

The thin-layer chromatographic tests were carried out using HPTLC precoated plates, silica gel 60 F 254, from E. Merck, Darmstadt.

The mixing ratios of solvents, mobile solvents or for chromatographic tests are in each case specified in volume/volume.

The analysis was carried out by mass spectroscopy and NMR.

### Preparation of substituted 1-oxa-2,8-diazaspiro[4.5]dec-2-ene derivatives

The synthesis of 1-oxa-2,8-diazaspiro[4.5]dec-2-ene-3,8-dicarboxylic acid-8-tert-butyl ester is illustrated in Diagram 3.

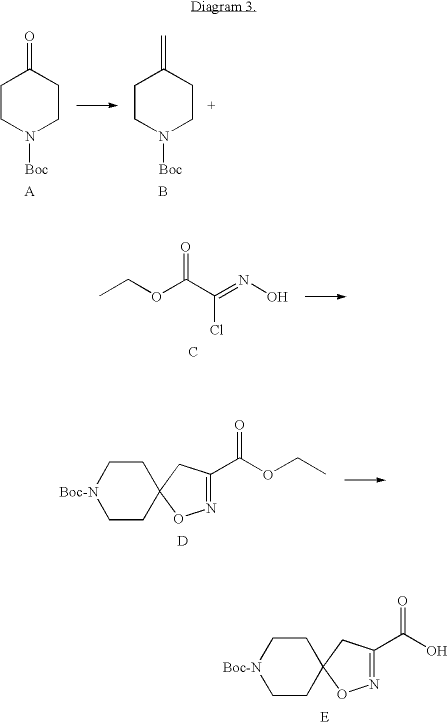

### Synthesis of 4-methylene piperidine-1-carboxylic acid tert-butyl ester (B)

1.6 g (14 mmol) potassium tert-butylate were added to a suspension of 5.34 g (15 mmol) methyltriphenylphosphonium bromide in 50 ml diethyl ether while stirring at 0° C. (ice bath). After stirring for 15 min, a solution of 2.00 g (10 mmol) 1-Boc-4-piperidone (A) in 15 ml diethyl ether was added slowly. The suspension was stirred for a further 30 min at 0° C. After the addition of 60 ml 10% aq. NH₄Cl solution, the organic phase was separated off, dried over magnesium sulphate and desolventised under vacuum. After chromatography on silica gel (hexane:EtOAc=5:1), 1.71 g (89%) 4-methylene piperidine-1-carboxylic acid tert-butyl ester (B) were obtained as a colourless liquid.

¹H-NMR spectrum (d₆-DMSO): δ=1.47 ppm (s, 9H, C(CH₃)₃); 2.16-2.19 ppm (m, 4H, CH₂); 3.40-3.44 ppm (m, 4H, CH₂); 4.74 (s, 2H, C═CH₂).

### Synthesis of 1-oxa-2,8-diazaspiro[4.5]dec-2-ene-3,8-dicarboxylic acid-8-tert-butyl ester 3-ethyl ester (D)

0.55 ml (3.9 mmol) of triethylamine which had been freshly distilled beforehand were slowly added to a mixture of 0.50 g (2.6 mmol) 4-methylene piperidine-1-carboxylic acid tert-butyl ester (B) and 0.60 g (3.9 mmol) 2-chloro-2-hydroxyiminoacetic acid ethyl ester (C) in 10 ml DCM at 0° C. (ice bath). After stirring for 12 h at RT, 0.79 g (5.1 mmol) of 2-chloro-2-hydroxyiminoacetic acid ethyl ester and 0.72 ml (5.1 mmol) of triethylamine were again added at 0° C. and the reaction mixture was stirred for a further 24 h. After washing with 10% aqueous citric acid and sat. aq. NaCl solution, a yellow oil was obtained after drying of the organic phase (MgSO₄) and removal of the solvent under vacuum. After column chromatography on silica gel (hexane:diethyl ether=4:1), 320 mg (39%) of 1-oxa-2,8-diazaspiro[4.5]dec-2-ene-3,8-dicarboxylic acid-8-tert-butyl ester 3-ethyl ester (D) were obtained in the form of a yellowish oil.

¹H-NMR spectrum (d₆-DMSO): δ=1.37 ppm (t, J=6.0 Hz, 3H, CH₃); 1.46 ppm (s, 9H, C(CH₃)₃); 1.67-1.75 ppm (m, 2H, CH₂); 1.85-1.92 ppm (m, 2H, CH₂); 2.96 ppm (s, 2H, CH₂); 3.39-3.49 ppm (m, 2H, CH₂); 3.60-3.70 ppm (m, 2H, CH₂); 4.35 (q, J=6.0 Hz, 2H, CH₂).

### Synthesis of 1-oxa-2,8-diazaspiro[4.5]dec-2-ene-3,8-dicarboxylic acid-8-tert-butyl ester (E)

A mixture of 320 mg (1 mmol) 4-methylene piperidine-1-carboxylic acid tert-butyl ester-3-ethyl ester (D) in 2 ml MeOH and 70 mg (1.5 mmol) lithium hydroxide monohydrate in 1.3 ml H₂O was stirred for 1.5 h at RT. After removal of the solvent under vacuum, the residue was taken up in water and EtOAc and separated, wherein the aq. phase was adjusted to pH=4 using citric acid. The organic phase was dried (MgSO₄) and desolventised under vacuum. 280 mg (98%) of the free acid 1-oxa-2,8-diazaspiro[4.5]dec-2-ene-3,8-dicarboxylic acid-8-tert-butyl ester (E) were obtained in the form of a colourless solid.

### Preparation of Substituted Spiro Compounds According to the Invention

The synthesis of substituted Spiro compounds according to the invention is illustrated in Diagram 4.

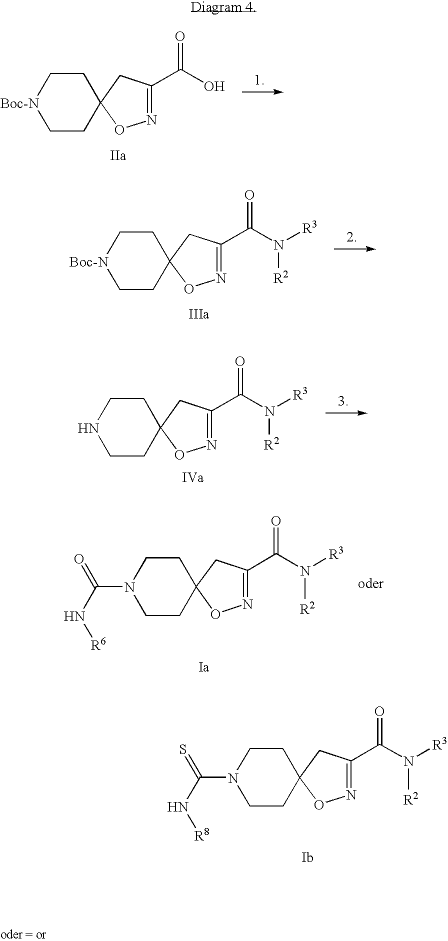

### Step 1: General Directions for Reacting 1-oxa-2,8-diazaspiro[4.5]dec-2-ene-3,8-dicarboxylic acid-8-tert-butyl ester of General Formula IIa with Primary or Secondary Amines of General Formula HNR²R³

A mixture of 1 equivalent of 1-oxa-2,8-diazaspiro[4.5]dec-2-ene-3,8-dicarboxylic acid-8-tert-butyl ester (E), 1 equivalent of the respective amine HNR²R³, 2.7 equivalents of N-methylmorpholine and 1.8 equivalents BOP in DMF was stirred for 12 h at RT. After removal of the DMF under vacuum, the residue was mixed with H₂O and EtOAc and separated. The organic phase was washed with H₂O, 10% aq. citric acid solution, sat. aq. Na₂CO₃ solution and sat. aq. NaCl solution, dried (MgSO₄) and desolventised under vacuum. After column chromatography (silica gel, diethyl ether:hexane=10:1), the respective coupling products were obtained.

In some cases, the coupling was carried out with 1 equivalent of 1-oxa-2,8-diazaspiro[4.5]dec-2-ene-3,8-dicarboxylic acid-8-tert-butyl ester (E), 1.05 equivalent of the respective amine and 1.05 equivalent N,N′-carbonyldiimidazole in THF at RT. For this purpose, the reaction mixture was stirred first for 90 minutes in the presence of the acid and of N,N′-carbonyldiimidazole and for a further 21 hours after the addition of the amine. For working up, the solvent was removed, the residue taken up in EtOAc and 10% aq. citric acid solution added. The organic phase was separated off, extracted with 10% citric acid solution, washed with sat. aq. NaHCO₃ solution and with sat. aq. NaCl solution. The organic phase was dried and the solvent removed under vacuum.

### Synthesis of 3-(3,4-dimethoxybenzylcarbamoyl)-1-oxa-2,8-diazaspiro[4.5]dec-2-ene-8-carboxylic acid tert-butyl ester

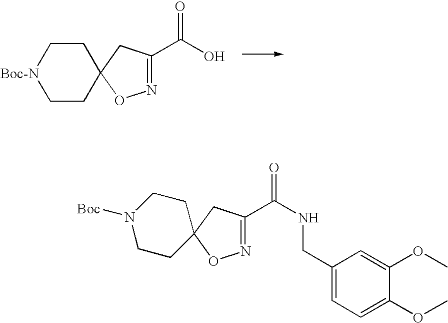

N,N′-carbonyldiimidazole (4.2 g, 26.06 mmol) was added to a solution of 1-oxa-2,8-diazaspiro[4.5]dec-2-ene-3,8-dicarboxylic acid-8-tert-butyl ester (7 g, 24.6 mmol) in abs. THF (100 ml) and stirred for 1.5 h at RT. After the addition of 3,4-dimethoxybenzylamine (4.35 g, 3.92 ml, 26.06 mmol), the mixture was stirred for a further 21 h. For working up, the mixture was concentrated, taken up in EtOAc (70 ml) and mixed with 10% aq. citric acid solution (40 ml). The organic phase was separated off, extracted with 10% aq. citric acid solution (40 ml), washed with sat. aq. NaHCO₃ solution (2×40 ml) and with sat. aq. NaCl solution (40 ml). The organic phase was dried and the solvent removed under vacuum. The amide 3-(3,4-dimethoxybenzylcarbamoyl)-1-oxa-2,8-diazaspiro[4.5]dec-2-ene-8-carboxylic acid tert-butyl ester was isolated as a beige-coloured solid in a yield of 80% (8.49 g) having a melting point of 118-120° C.

### Synthesis of 3-(4-tert-butylbenzylcarbamoyl)-1-oxa-2,8-diazaspiro[4.5]dec-2-ene-8-carboxylic acid tert-butyl ester

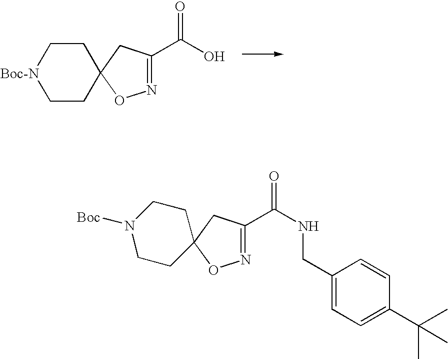

4-Methylmorpholine (1.16 mL, 10.5 mmol) and 4-(tert-butyl)benzylamine (0.574 g, 3.5 mmol) were added to a solution of 1-oxa-2,8-diazaspiro[4.5]dec-2-ene-3,8-dicarboxylic acid-8-tert-butyl ester (1 g, 3.5 mmol) in abs. DMF (20 mL) and the reaction mixture was stirred. BOP (2.02 g, 4.57 mmol) was added and the mixture stirred overnight. For working up, the mixture was concentrated, taken up in EtOAc (70 ml) and sat. aq. NaHCO₃ solution (2×40 ml). The phases were separated and the aqueous phase was washed with acetic ester. The combined organic phases were washed with sat. aq. NaCl solution (40 ml), dried and the solvent was removed under vacuum. The amide 3-(4-tert-butylbenzylcarbamoyl)-1-oxa-2,8-diazaspiro[4.5]dec-2-ene-8-carboxylic acid tert-butyl ester was isolated after column chromatography (SiO₂, EE/hexane 1:2) as a solid in a yield of 84% (1.27 g).

### Synthesis of 3-(4-trifluoromethylbenzylcarbamoyl)-1-oxa-2,8-diazaspiro[4.5]dec-2-ene-8-carboxylic acid tert-butyl ester

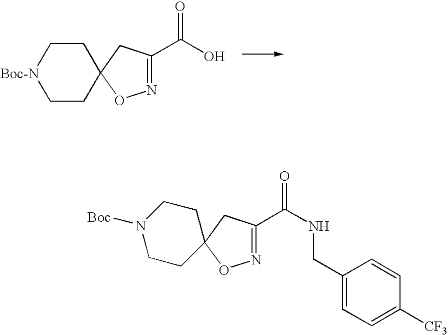

4-Methylmorpholine (1.16 mL), 10.5 mmol) and 4-(trifluoromethyl)benzylamine (0.574 g, 3.5 mmol) were added to a solution of 1-oxa-2,8-diazaspiro[4.5]dec-2-ene-3,8-dicarboxylic acid-8-tert-butyl ester (1 g, 3.5 mmol) in abs. DMF (20 mL) and the reaction mixture was stirred. BOP (2.02 g, 4.57 mmol) was added and the mixture stirred overnight. For working up, the mixture was concentrated, taken up in EtOAc (70 ml) and sat. aq. NaHCO₃ solution (2×40 ml). The phases were separated and the aqueous phase washed with acetic ester. The combined organic phases were washed with sat. aq. NaCl solution (40 ml), dried and the solvent was removed under vacuum. The amide 3-(4-trifluoromethylbenzylcarbamoyl)-1-oxa-2,8-diazaspiro[4.5]dec-2-ene-8-carboxylic acid tert-butyl ester was isolated after column chromatography (SiO₂, ether/hexane 10:1) as a solid in a yield of 84% (1.31 g).

### Synthesis of 3-(5-chloropyridin-2-ylcarbamoyl)-1-oxa-2,8-diazaspiro[4.5]dec-2-ene-8-carboxylic acid tert-butyl ester

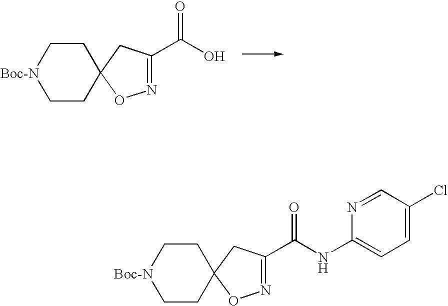

N,N′-carbonyldiimidazole (5.8 g, 36.9 mmol) was added to a solution of 1-oxa-2,8-diazaspiro[4.5]dec-2-ene-3,8-dicarboxylic acid-8-tert-butyl ester (7 g, 24.6 mmol) in abs. THF (100 ml) and the mixture stirred for 2 h at RT. After the addition of 2-amino-5-chloropyridine (4.74 g, 36.9 mmol), the reaction mixture was stirred for 45 h. For working up, the mixture was concentrated, taken up in EtOAc (120 ml) and mixed with 10% aq. citric acid solution (100 ml). Between the phases there remained undissolved a secondary product which was separated off by filtration. The organic phase of the filtrate was separated off, extracted with 10% aq. citric acid solution (50 ml), washed with sat. aq. NaHCO₃ solution (2×50 ml) and sat. aq. NaCl solution (50 ml). The organic phase was dried and the solvent removed under vacuum. The amide 3-(5-chloropyridin-2-ylcarbamoyl)-1-oxa-2,8-diazaspiro[4.5]dec-2-ene-8-carboxylic acid tert-butyl ester was isolated as a brown solid crude product (7.62 g). After chromatographic purification [silica gel 60 (150 g); EtOAc/cyclohexane 1:3 (1.8 I)], the amide was isolated in a yield of 70% (6.8 g) having a melting point of 171-172° C.

### Synthesis of 3-[4-(3-chloropyridin-2-yl)piperazine-1-carbonyl]-1-oxa-2,8-diazaspiro[4.5]dec-2-ene-8-carboxylic acid tert-butyl ester

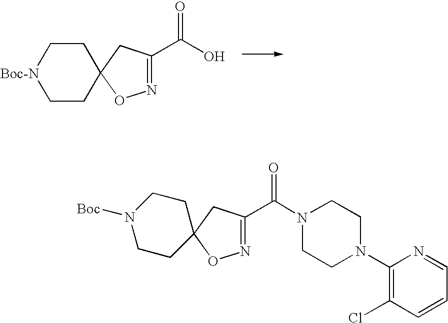

N,N′-carbonyldiimidazole (4.39 g, 27.1 mmol) was added to a solution of 1-oxa-2,8-diazaspiro[4.5]dec-2-ene-3,8-dicarboxylic acid-8-tert-butyl ester (7 g, 24.6 mmol) in abs. THF (80 ml). The mixture was stirred for 1.5 h at RT. After the addition of 1-(3-chloro-2-pyridinyl)piperazine (5.36 g, 27.1 mmol) in abs. THF (20 ml), the mixture was stirred for 6 days at RT. For working up, the solvent was removed under vacuum. The brown oily residue was taken up in EtOAc (80 ml) and stirred with 10% aq. citric acid solution (80 ml) for 20 min. The phases were separated. The organic phase was washed with sat. aq. NaHCO₃ solution (2×40 ml) and sat. aq. NaCl solution (40 ml), dried and the solvent removed under vacuum. The amide 3-[4-(3-chloropyridin-2-yl)piperazine-1-carbonyl]-1-oxa-2,8-diazaspiro[4.5]dec-2-ene-8-carboxylic acid tert-butyl ester was obtained as a viscous brown oil in a yield of 72% (8.25 g).

### Synthesis of 3-[4-(3-chloropyridin-2-yl)-2-methylpiperazine-1-carbonyl]-1-oxa-2,8-diazaspiro[4.5]dec-2-ene-8-carboxylic acid tert-butyl ester

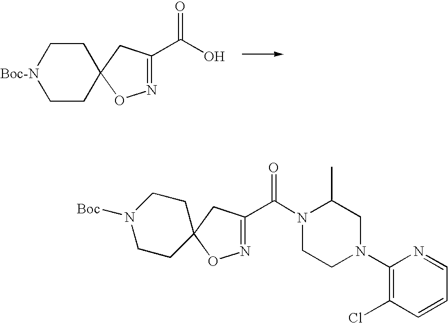

TBTU (2.8 g, 8.8 mmol), HOBt (1.18 g, 8.8 mmol), diisopropylethylamine (5 mL, 26.4 mmol) and 1-(3-trifluoromethyl-2-pyridinyl)-3-methylpiperazine (1.86 g, 8.8 mmol) were added to a solution of 1-oxa-2,8-diazaspiro[4.5]dec-2-ene-3,8-dicarboxylic acid-8-tert-butyl ester (2.5 g, 8.8 mmol) in abs. THF (67 ml). The reaction mixture was stirred overnight at RT, DMF (20 mL) added and the mixture stirred for a further 6 hours. For working up, the solvent was removed under vacuum. The brown oily residue was taken up in EtOAc (200 mL) and washed with sat. aq. NaHCO₃ solution (200 mL), sat. aq. NaCl solution (200 ml), sat. aq. NaCl solution (200 mL) and sat. aq. NH₄HSO₄ solution (200 mL), dried and the solvent removed under vacuum. The amide 3-[4-(3-chloropyridin-2-yl)-2-methylpiperazine-1-carbonyl]-1-oxa-2,8-diazaspiro[4.5]dec-2-ene-8-carboxylic acid tert-butyl ester was obtained in a yield of 60% (2.5 g).

### Synthesis of 3-[(5-methylfuran-2-ylmethyl)carbamoyl]-1-oxa-2,8-diazaspiro[4.5]dec-2-ene-8-carboxylic acid tert-butyl ester

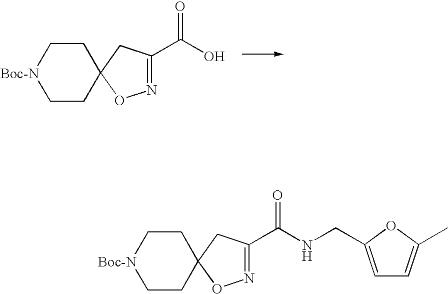

N,N′-carbonyldiimidazole (4.39 g, 27.1 mmol) was added to a solution of 1-oxa-2,8-diazaspiro[4.5]dec-2-ene-3,8-dicarboxylic acid-8-tert-butyl ester (7 g, 24.6 mmol) in abs. THF (80 ml) and the mixture stirred for 2.5 h at RT. After the addition of 5-methylfuranyl-2-methylamine (3.01 g, 27.1 mmol) in abs. THF (20 ml), the reaction mixture was stirred for 4 days at RT. For working up, the solvent was removed under vacuum. The light brown oily residue was taken up in EtOAc (80 ml) and stirred with 10% aq. citric acid solution (80 ml) for 20 min. The phases were separated. The organic phase was washed with sat. aq. NaHCO₃ solution (2×40 ml) and sat. aq. NaCl solution (40 ml), dried and the solvent removed under vacuum. The amide 3-[(5-methylfuran-2-ylmethyl)carbamoyl]-1-oxa-2,8-diazaspiro[4.5]dec-2-ene-8-carboxylic acid tert-butyl ester was obtained as a light yellow solid in a yield of 98% (9.1 g).

### Synthesis of 3-(4-chlorobenzylcarbamoyl)-1-oxa-2,8-diazaspiro[4.5]dec-2-ene-8-carboxylic acid tert-butyl ester

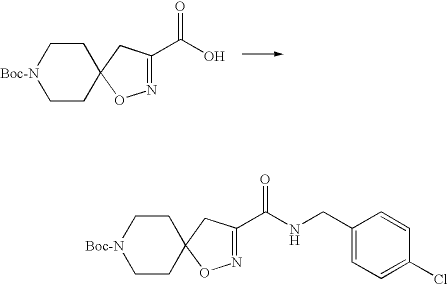

N,N′-carbonyldiimidazole (4.39 g, 27.1 mmol) was added to a solution of 1-oxa-2,8-diazaspiro[4.5]dec-2-ene-3,8-dicarboxylic acid-8-tert-butyl ester (7 g, 24.6 mmol) in abs. THF (100 ml). The reaction mixture was stirred for 1.5 h at RT. After the addition of 4-chlorobenzylamine (4.22 g, 29.8 mmol), the reaction mixture was stirred for 3 days at RT. For working up, the solvent was removed under vacuum. The brown oily residue was taken up in EtOAc (80 ml) and stirred with 10% aq. citric acid solution (80 ml) 20 min. The phases were separated. The organic phase was washed with sat. aq. NaHCO₃ solution (2×40 ml) and sat. aq. NaCl solution (40 ml), dried and the solvent removed under vacuum. The amide 3-(4-chlorobenzylcarbamoyl)-1-oxa-2,8-diazaspiro[4.5]dec-2-ene-8-carboxylic acid tert-butyl ester was obtained as a light brown solid in a yield of 97% (9.74 g) having a melting point of 136-138° C.

### Synthesis of 3-(4-hydroxy-3-methoxybenzylcarbamoyl)-1-oxa-2,8-diazaspiro[4.5]dec-2-ene-8-carboxylic acid tert-butyl ester

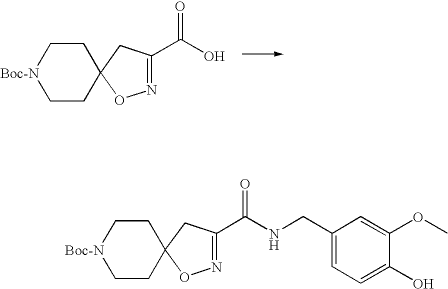

BOP (11.72 g, 26.53 mmol) and N-methylmorpholine (6.73 g, 61.23 mmol) were added to a solution of 1-oxa-2,8-diazaspiro[4.5]dec-2-ene-3,8-dicarboxylic acid-8-tert-butyl ester (5.8 g, 20.4 mmol) in anhydrous DMF (60 ml). The mixture was stirred for 1.5 h at RT under argon. After the addition of 4-hydroxy-3-methoxybenzylamine (4.69 g, 30.6 mmol), the reaction mixture was stirred for 22.5 h at RT. For working up, the solvent was removed under vacuum. The brown oily residue was taken up in water (100 ml), sat. aq. NaHCO₃ solution (60 ml) and DCM (100 ml). The phases were separated and the aqueous phase extracted with DCM (3×100 ml). The organic phase was dried and the solvent removed under vacuum. The brown oily residue was taken up again in DCM (70 ml) and washed with water (2×35 ml). The phases were separated. The organic phase was dried and the solvent removed under vacuum. Subsequently, the residue was taken up in water (150 ml) and stirred and the precipitated solid removed by suction-filtration. The amide 3-(4-hydroxy-3-methoxybenzylcarbamoyl)-1-oxa-2,8-diazaspiro[4.5]dec-2-ene-8-carboxylic acid tert-butyl ester was obtained as a light brown solid in a yield of 99% (8.47 g) having a melting point of 151-157° C.

### Synthesis of 3-[4-(3-trifluoromethylpyridin-2-yl)piperazine-1-carbonyl]-1-oxa-2,8-diazaspiro[4.5]dec-2-ene-8-carboxylic acid tert-butyl ester

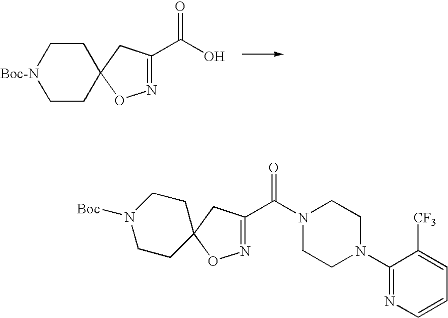

4-Methylmorpholine (1.75 mL, 15.81 mmol), BOP (3.03 g, 6.9 mmol) and 1-(3-trifluoromethyl-2-pyridinyl)piperazine were added to a solution of 1-oxa-2,8-diazaspiro[4.5]dec-2-ene-3,8-dicarboxylic acid-8-tert-butyl ester (1.5 g, 5.2 mmol) in abs. DMF (25 ml) and the reaction mixture was stirred overnight at RT. For working up, the solvent was removed under vacuum. The brown oily residue was taken up in EtOAc (40 ml) and sat. aq. NaHCO₃ solution (2×20 ml). The phases were separated. The aqueous phase was washed with EtOAc and the combined organic phase was washed with sat. aq. NH₄Cl solution (2×20 ml) and sat. aq. NaCl solution (20 ml), dried and the solvent removed under vacuum. The residue was taken up in acetic ester and purified by column chromatography (SiO₂, EE/hexane 1:1). The desired product was obtained in a yield of 2.18 g.

### Step 2: General Directions for Cleavage of the Boc Group from Compounds of General Formula IIIa

The corresponding N-Boc-piperidine of general formula IIIa in MeOH was mixed with an excess of a 5 N solution of HCl in isopropanol at RT and stirred. Once the reaction had been completed, the solution was concentrated until the mixture started to turn cloudy, then mixed with diethyl ether and stored overnight until completion of the precipitation of the desired product as a hydrochloride at 4° C. The precipitated solid was filtered out, washed with small portions of diethyl ether and dried under vacuum.

### Synthesis of 1-oxa-2,8-diazaspiro[4.5]dec-2-ene-3-carboxylic acid-3,4-dimethoxybenzylamide

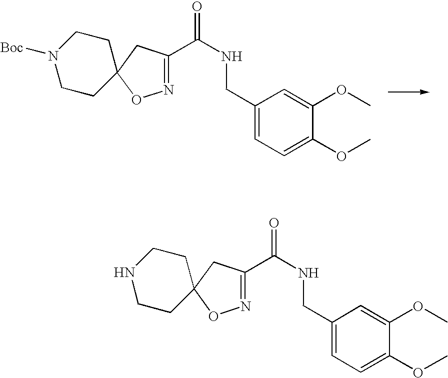

A solution of 3-(3,4-dimethoxybenzylcarbamoyl)-1-oxa-2,8-diazaspiro[4.5]dec-2-ene-8-carboxylic acid tert-butyl ester (8.4 g, 19.38 mmol) in MeOH (300 ml) was mixed with 5 N HCl (80 ml, 400 mmol) in isopropanol and stirred for 3 d at RT. There formed a portion of the hydrochloride of the amine 1-oxa-2,8-diazaspiro[4.5]dec-2-ene-3-carboxylic acid-3,4-dimethoxybenzylamide as a colourless solid (4.2 g). The filtrate was concentrated to 80 ml and further hydrochloride isolated (2.29 g). The two fractions were combined and the hydrochloride was obtained in a yield of 92% (mp. 132-136° C.). To liberate the base, the hydrochloride was taken up in DCM (100 ml) and sat. aq. NaHCO₃ solution and stirred for 1 h at RT. The aqueous phase was separated off and extracted with DCM (4×50 ml). The combined organic phases were dried and the solvent was removed under vacuum. The amine 1-oxa-2,8-diazaspiro[4.5]dec-2-ene-3-carboxylic acid-3,4-dimethoxybenzylamide was isolated as a colourless solid having a melting point of 121-123° C. in a yield of 89% (5.68 g).

### Synthesis of 1-oxa-2,8-diazaspiro[4.5]dec-2-ene-3-carboxylic acid-4-trifluoromethylbenzylamide hydrochloride

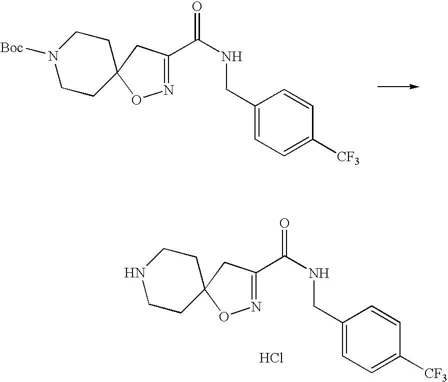

A solution of 3-(4-trifluoromethylbenzylcarbamoyl)-1-oxa-2,8-diazaspiro[4.5]dec-2-ene-8-carboxylic acid tert-butyl ester (1 g, 3.6 mmol) in methanolic HCl (4 N, 5 mL) was stirred at RT. The mixture was made up twice with conc. aq. HCl (1 mL) and once with MeOH (5 mL). The solvent was removed under vacuum and the residue taken up in ether. The hydrochloride of the amine 1-oxa-2,8-diazaspiro[4.5]dec-2-ene-3-carboxylic acid-4-trifluoromethylbenzylamide formed as a colourless solid (1.31 g, 85%).

### Synthesis of 1-oxa-2,8-diazaspiro[4.5]dec-2-ene-3-carboxylic acid-4-tert-butylbenzylamide hydrochloride

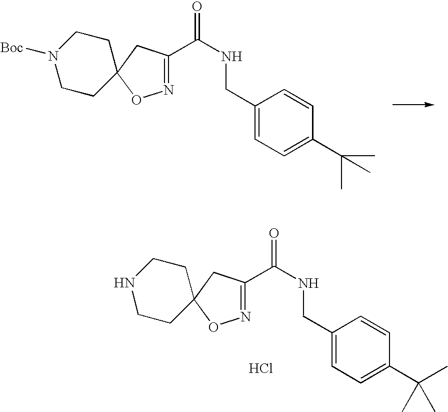

A solution of 3-(4-tert-butylbenzylcarbamoyl)-1-oxa-2,8-diazaspiro[4.5]dec-2-ene-8-carboxylic acid tert-butyl ester (0.55 g, 1.28 mmol) in methanol (10 mL) was stirred at RT for 48 hours after the addition of conc. aq. HCl (2.2 mL). The solvent was removed under vacuum and the residue taken up in ether. The hydrochloride of the amine 1-oxa-2,8-diazaspiro[4.5]dec-2-ene-3-carboxylic acid-4-tert-butylbenzylamide formed as a colourless solid (0.32 g, 69%).

### Synthesis of 1-oxa-2,8-diazaspiro[4.5]dec-2-ene-3-carboxylic acid-4-chlorobenzylamide hydrochloride and N-(5-chloropyridin-2-yl)-1-oxa-2,8-diazaspiro[4.5]dec-2-ene-3-imido acid methyl ester dihydrochloride

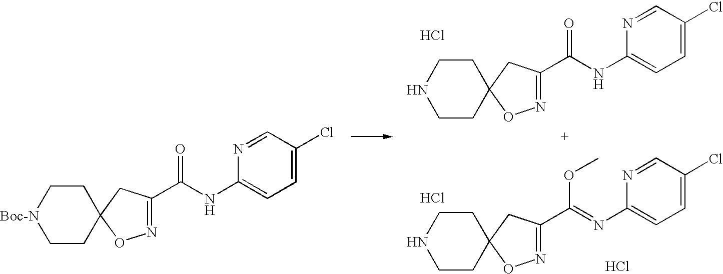

A solution of 3-(5-chloropyridin-2-ylcarbamoyl)-1-oxa-2,8-diazaspiro[4.5]dec-2-ene-8-carboxylic acid tert-butyl ester (6.8 g, 17.2 mmol) in abs. MeOH (600 ml) was mixed with 5 N hydrochloric acid (69 ml, 344 mmol) in isopropanol and stirred for 3 d at RT. The mixture was concentrated to 50 ml, wherein 1-oxa-2,8-diazaspiro[4.5]dec-2-ene-3-carboxylic acid-4-chlorobenzylamide hydrochloride formed as a colourless solid (1.9 g, 33%, mp. 280-284° C.). The filtrate was concentrated and the oily residue taken up in MeOH (30 ml). After the addition of diethyl ether (200 ml), the mixture was stirred for 16 h at RT. The imido ester N-(5-chloropyridin-2-yl)-1-oxa-2,8-diazaspiro[4.5]dec-2-ene-3-imido acid methyl ester dihydrochloride formed as a colourless solid and could be obtained in a yield of 63% (4.12 g).

### Synthesis of 1-oxa-2,8-diazaspiro[4.5]dec-2-ene-3-carboxylic acid-4-chlorobenzylamide trifluoroacetate

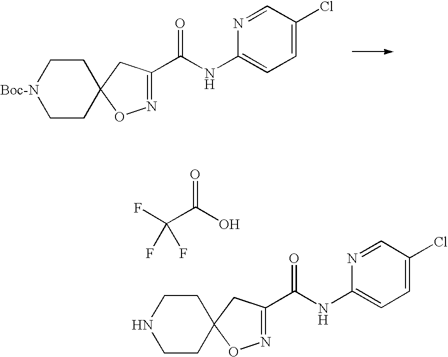

Trifluoroacetic acid (20 ml) was slowly added to a solution of 3-(5-chloropyridin-2-ylcarbamoyl)-1-oxa-2,8-diazaspiro[4.5]dec-2-ene-8-carboxylic acid tert-butyl ester (4.59 g, 11.6 mmol) in DCM (30 ml). The reaction solution was stirred for 3 d at RT. The mixture was concentrated, the oily residue mixed with diethyl ether (100 ml) and stirred for 2 h. 1-Oxa-2,8-diazaspiro[4.5]dec-2-ene-3-carboxylic acid-4-chlorobenzylamide trifluoroacetate was isolated as a colourless solid in a yield of 99% (4.65 g) having a melting point of 232-234° C.

### Synthesis of [4-(3-chloropyridin-2-yl)piperazin-1-yl]-(1-oxa-2,8-diazaspiro[4.5]dec-2-en-3-yl)methanone hydrochloride

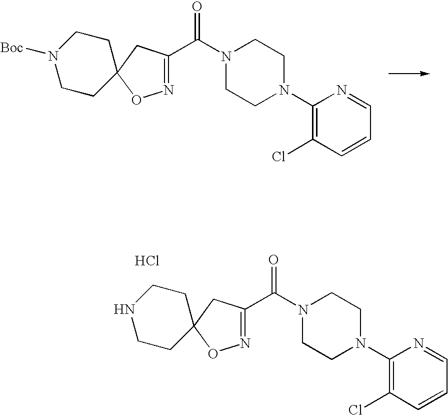

A solution of 3-[4-(3-chloropyridin-2-yl)piperazine-1-carbonyl]-1-oxa-2,8-diazaspiro[4.5]dec-2-ene-8-carboxylic acid tert-butyl ester (8.25 g, 17.8 mmol) in abs. MeOH (300 ml) was mixed with 5.5 N HCl in isopropanol (64.7 ml, 356 mmol) and stirred for 22 h at RT. The mixture was removed under vacuum on the addition of 10 ml of solvent. Diethyl ether (10 ml) was slowly added to the cooled solution until the mixture turned cloudy. The solution was stirred for 1.5 h at RT, wherein the [4-(3-chloropyridin-2-yl)piperazin-1-yl]-(1-oxa-2,8-diazaspiro[4.5]dec-2-en-3-yl)methanone hydrochloride formed as a colourless solid (6.22 g, 88%, mp 135-138° C.).

### Synthesis of [4-(3-chloropyridin-2-yl)-piperazin-1-yl]-(1-oxa-2,8-diazaspiro[4.5]dec-2-en-3-yl)methanone hydrochloride

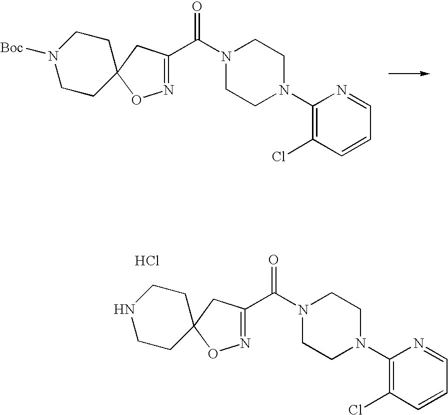

A solution of 3-[4-(3-chloropyridin-2-yl)piperazine-1-carbonyl]-1-oxa-2,8-diazaspiro[4.5]dec-2-ene-8-carboxylic acid tert-butyl ester (2.5 g, 5.23 mmol) in abs. MeOH (50 mL) was mixed with 1.25 N HCl in isopropanol (34.3 ml, 43 mmol) while being cooled with ice and stirred for 5 h at RT. The solvent was removed under vacuum. Diethyl ether (10 ml) was slowly added to the cooled solution until the mixture turned cloudy. The solution was stirred for 1.5 h at RT, wherein the [4-(3-chloropyridin-2-yl)piperazin-1-yl]-(1-oxa-2,8-diazaspiro[4.5]dec-2-en-3-yl)methanone hydrochloride formed as a colourless solid (2.4 g).

### Synthesis of [4-(3-trifluoromethylpyridin-2-yl)piperazin-1-yl]-(1-oxa-2,8-diazaspiro[4.5]dec-2-en-3-yl)methanone hydrochloride

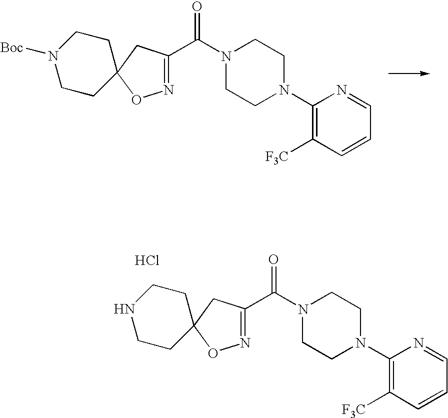

A solution of 3-[4-(3-trifluoromethylpyridin-2-yl)piperazine-1-carbonyl]-1-oxa-2,8-diazaspiro[4.5]dec-2-ene-8-carboxylic acid tert-butyl ester (2.18 g, 4.3 mmol) in abs. MeOH (40 ml) was mixed with 1.25 N HCl in isopropanol (29 mL, 36 mmol) while being cooled with ice and stirred overnight at RT. The solvent was removed under vacuum, the residue taken up in ether (80 mL) and stirred for 20 min at RT, wherein the [4-(3-trifluoromethylpyridin-2-yl)piperazin-1-yl]-(1-oxa-2,8-diazaspiro[4.5]dec-2-en-3-yl)methanone hydrochloride formed as a colourless solid (1.8 g, 97%).

### Synthesis of 1-oxa-2,8-diazaspiro[4.5]dec-2-ene-3-carboxylic acid-(5-methylfuran-2-ylmethyl)amide as a hydrochloride salt

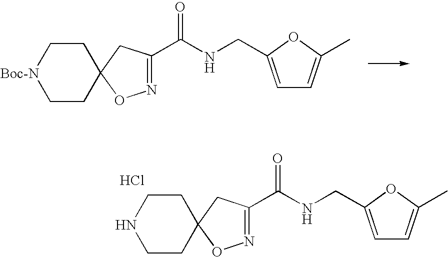

A solution of 3-[(5-methylfuran-2-ylmethyl)carbamoyl]-1-oxa-2,8-diazaspiro[4.5]dec-2-ene-8-carboxylic acid tert-butyl ester (9.2 g, 24.3 mmol) in abs. MeOH (300 ml) was mixed with 5.5 N HCl in isopropanol (88.6 ml, 487.5 mmol) and stirred for 24 h at RT. The mixture was concentrated to half its original volume, wherein a colourless solid formed. The suspension was subsequently stirred for 30 min at RT and the solid which formed was then removed by suction-filtration. The hydrochloride of 1-oxa-2,8-diazaspiro[4.5]dec-2-ene-3-carboxylic acid-(5-methylfuran-2-ylmethyl)amide was obtained as a colourless solid (5.3 g, 70%) having a melting point of 218-220° C.

### Synthesis of 1-oxa-2,8-diazaspiro[4.5]dec-2-ene-3-carboxylic acid-4-chlorobenzylamide as a hydrochloride salt

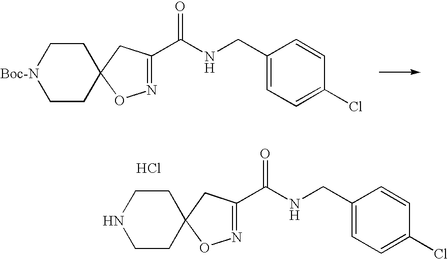

A solution of 3-(4-chlorobenzylcarbamoyl)-1-oxa-2,8-diazaspiro[4.5]dec-2-ene-8-carboxylic acid tert-butyl ester (9.7 g, 23.7 mmol) in abs. MeOH (350 ml) was mixed with 5,5 N HCl in isopropanol (86.2 ml, 474 mmol) and stirred for 20 h at RT. The mixture was concentrated to half its original volume, wherein a colourless solid formed. The suspension was subsequently stirred for 30 min at RT and then removed by suction-filtration. The hydrochloride of 1-oxa-2,8-diazaspiro[4.5]dec-2-ene-3-carboxylic acid-4-chlorobenzylamide could be obtained as a colourless solid (5.95 g, 73%) having a melting point of 269-273° C.

### Synthesis of 1-oxa-2,8-diazaspiro[4.5]dec-2-ene-3-carboxylic acid-4-hydroxy-3-methoxybenzylamide as a hydrochloride salt

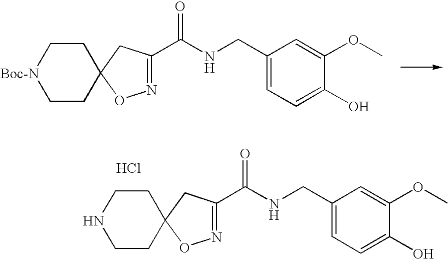

A solution of 3-(4-hydroxy-3-methoxybenzylcarbamoyl)-1-oxa-2,8-diazaspiro[4.5]dec-2-ene-8-carboxylic acid tert-butyl ester (8.27 g, 19.7 mmol) in abs. MeOH (650 ml) was mixed with 5.5 N HCl in isopropanol (71.7 ml, 394.3 mmol) and stirred for 18 h at RT. A colourless solid formed and was removed by suction-filtration. The mixture was concentrated, wherein a colourless solid again formed. The hydrochloride of 1-oxa-2,8-diazaspiro[4.5]dec-2-ene-3-carboxylic acid-4-hydroxy-3-methoxybenzylamide was obtained in a yield of 68% (4.75 g) having a melting point of 264-269° C.

### Step 3: General Directions for Reacting Amines of General Formula IVa with Isocyanates or Thioisocyanates of General Formula R⁶—N═C═O or R⁸—N═C═S

a. Manual Synthesis

The amines of general formula IVa (1 equivalent) were dissolved in toluene (150 equivalents) or DMF (25 equivalents). The isocyanate or thioisocyanate of general formula R⁶—N═C═O or R⁸—N═C═S (1 equivalent) and triethylamine (1 equivalent) were added and the reaction mixture was heated to boiling point. After 3 hours, EtOAc and sat. aq. NaHCO₃ solution were added and the phases separated. The aqueous phase was extracted several times with EtOAc. The combined organic phases were washed with water and sat. aq. NaCl solution, dried over magnesium sulphate and the solvent was removed under vacuum. The residue was separated by column chromatography (silica gel, hexane/EtOAc).

The following substituted spiro compound according to the invention was prepared as described under 3a.

### Preparation of 3-[4-(3-chloropyridin-2-yl)piperazine-1-carbonyl]-1-oxa-2,8-diazaspiro[4.5]dec-2-ene-8-carboxylic acid-(4-tert-butylphenyl)amide

The compound [4-(3-chloropyridin-2-yl)piperazin-1-yl]-(1-oxa-2,8-diazaspiro[4.5]dec-2-en-3-yl)methanone hydrochloride (1 g, 2.50 mmol) was dissolved in toluene (40 ml) and mixed with tert-butylphenyl isocyanate (438 mg, 2.50 mmol) and triethylamine (350 μl). The reaction mixture was heated to boiling point for 3 hours. The precipitate formed was removed by suction-filtration, dried and taken up in EtOAc and sat. aq. NaHCO₃ solution. The phases were separated and the aqueous phase was extracted with EtOAc. The combined organic phases were dried over magnesium sulphate and the solvent was removed under vacuum. The residue was dried and purified by column chromatography (silica gel, hexane/EtOAc 1:1).

b. Automated Synthesis

There were first produced the following parent solutions:
Solution I: 0.05 M solution of the amine of general formula IVa in toluene
Solution II: 0.1 M solution of the isocyanate of general formula R⁶—N═C═O or the isothiocyanate of general formula R⁸—N═C═S in toluene

Solution I (2 mL) was placed in a dry threaded glass container with a septum cap at RT and mixed with solution II (1 mL). The reaction mixture was stirred under reflux 6 h in a strike reactor. The reaction mixture was transferred to vials and the solvent removed using GeneVac equipment.

The following isocyanates of general formula R⁶—N═C═O were used for synthesis: phenyl isocyanate, 2-methylphenyl isocyanate, m-tolyl isocyanate, p-tolyl isocyanate, 2-ethylphenyl isocyanate, 3-ethylphenyl isocyanate, 4-ethylphenyl isocyanate, 2-propylphenyl isocyanate, 2-fluorophenyl isocyanate, 3-fluorophenyl isocyanate, 4-fluorophenyl isocyanate, 2-chlorophenyl isocyanate, 3-chlorophenyl isocyanate, 4-chlorophenyl isocyanate, 2-bromophenyl isocyanate, 3-bromophenyl isocyanate, 4-bromophenyl isocyanate, 3-iodophenyl isocyanate, 4-iodophenyl isocyanate, 2-methoxyphenyl isocyanate, 3-methoxyphenyl isocyanate, 4-methoxyphenyl isocyanate, 2-ethoxyphenyl isocyanate, 4-ethoxyphenyl isocyanate, 2-(methylthio)phenyl isocyanate, 3-(methylthio)phenyl isocyanate, 4-(methylthio)phenyl isocyanate, 2-isopropylphenyl isocyanate, 4-isopropylphenyl isocyanate, 4-butylphenyl isocyanate, 3-cyanophenyl isocyanate, 2-methoxycarbonylphenyl isocyanate, 3-methoxycarbonylphenyl isocyanate, ethyl-2-isocyanatobenzoate, 3-ethoxycarbonylphenyl isocyanate, 4-ethoxycarbonylphenyl isocyanate, 2-(trifluoromethyl)phenyl isocyanate, 3-(trifluoromethyl)phenyl isocyanate, 4-(trifluoromethyl)phenyl isocyanate, 1-naphthyl isocyanate, 2-biphenyl isocyanate, 4-biphenyl isocyanate, 2-phenoxyphenyl isocyanate, 4-phenoxyphenyl isocyanate, 4-benzyloxyphenyl isocyanate, 4-(dimethylamino)phenyl isocyanate, 2,6-difluorophenyl isocyanate, 2,5-difluorophenyl isocyanate, 2,4-difluorophenyl isocyanate, 3,4-difluorophenyl isocyanate, 2,6-dichlorophenyl isocyanate, 2,3-dichlorophenyl isocyanate, 2,5-dichlorophenyl isocyanate, 3,5-dichlorophenyl isocyanate, 2,4-dichlorophenyl isocyanate, 3,4-dichlorophenyl isocyanate, 2,4-dibromophenyl isocyanate, 2-chloro-5-(trifluoromethyl)phenyl isocyanate, 4-chloro-2-(trifluoromethyl)phenyl isocyanate, 4-chloro-3-(trifluoromethyl)phenyl isocyanate, 4-bromo-2-(trifluoromethyl)phenyl isocyanate, 3,5-bis-(trifluoromethyl)phenyl isocyanate, 2-(trifluoromethoxy)phenyl isocyanate, 2,4-dimethoxyphenyl isocyanate, 2,5-dimethoxyphenyl isocyanate, 3,5-dimethoxyphenyl isocyanate, 2-fluoro-5-methylphenyl isocyanate, 3-fluoro-4-methylphenyl isocyanate, 3-chloro-2-methylphenyl isocyanate, 4-chloro-2-methylphenyl isocyanate, 5-chloro-2-methylphenyl isocyanate, 3-chloro-4-methylphenyl isocyanate, 4-bromo-2-methylphenyl isocyanate, 3-chloro-4-fluorophenyl isocyanate, 4-bromo-2-fluorophenyl isocyanate, 3,5-dimethylphenyl isocyanate, 2,6-dimethylphenyl isocyanate, 3,4-dimethylphenyl isocyanate, 2,5-dimethylphenyl isocyanate, 2,4-dimethylphenyl isocyanate, 2-ethyl-6-methylphenyl isocyanate, 2-isopropyl-6-methylphenyl isocyanate, 2-tert-butyl-6-methylphenyl isocyanate, 2,6-diethylphenyl isocyanate, 2-ethyl-6-isopropylphenyl isocyanate, 2,6-diisopropylphenyl isocyanate, 4-methoxy-2-methylphenyl isocyanate, 2-methoxy-5-methylphenyl isocyanate, 5-chloro-2-methoxyphenyl isocyanate, 4-bromo-2,6-dimethylphenyl isocyanate, 2,4,5-trichlorophenyl isocyanate, 2,4-dibromo-6-fluorophenyl isocyanate, 2-bromo-4,6-difluorophenyl isocyanate, 5-chloro-2,4-dimethoxyphenyl isocyanate, 3,4,5-trimethoxyphenyl isocyanate, 2,4,5-trimethylphenyl isocyanate, 2,4,6-trimethylphenyl isocyanate, 4-trifluoromethoxyphenyl isocyanate, n-propyl isocyanate, n-butyl isocyanate, cyclohexyl isocyanate, 2-chlorethyl isocyanate, 3-chlorpropyl isocyanate, 2-bromoethyl isocyanate, benzyl isocyanate, phenylethyl isocyanate, 3-methylbenzyl isocyanate, 4-methylbenzyl isocyanate, 2-ethylbenzyl isocyanate, 3-ethylbenzyl isocyanate, 4-ethylbenzyl isocyanate, 4-fluorobenzyl isocyanate, 2-chlorobenzyl isocyanate, 1-(4-bromophenyl)ethyl isocyanate, 2,4-dichlorobenzyl isocyanate, 3,4-dichlorobenzyl isocyanate, 4-methoxybenzyl isocyanate, 1-(1-naphthyl)ethyl isocyanate, 2-methylbenzyl isocyanate, n-pentyl isocyanate, 4-tert-butylphenyl isocyanate.

The following isothiocyanates of general formula R⁸—N═C═S were used for synthesis: methyl isothiocyanate, ethyl isothiocyanate, n-propyl isothiocyanate, n-butyl isothiocyanate, n-pentyl isothiocyanate, n-hexyl isothiocyanate, n-heptyl isothiocyanate, n-octyl isothiocyanate, n-nonyl isothiocyanate, n-decyl isothiocyanate, n-dodecyl isothiocyanate, n-tetradecyl isothiocyanate, n-octadecyl isothiocyanate, isopropyl isothiocyanate, isobutyl isothiocyanate, tert-butyl isothiocyanate, tert-amyl isothiocyanate, allyl isothiocyanate, methallyl isothiocyanate, chloromethyl isothiocyanate, 2-chlorethyl isothiocyanate, 2-methoxyethyl isothiocyanate, 3-ethoxypropyl isothiocyanate, isothiocyanatoacetic acid methyl ester, 2-isothiocyanatopropionic acid methyl ester, ethyl-3-isothiocyanatopropionate, ethyl-2-isothiocyanatopropionate, ethyl-3-isothiocyanatobutyrate, 3-(diethylamino)propyl isothiocyanate, cyclopropyl isothiocyanate, cyclopentyl isothiocyanate, cyclohexyl isothiocyanate, cyclohexylmethyll isothiocyanate, cyclooctyl isothiocyanate, cyclododecyl isothiocyanate, 1-adamantyl isothiocyanate, 2-(isothiocyanatomethyl)tetrahydrofuran, 2-(4-morpholino)ethyl isothiocyanate, 3-(4-morpholino)propyl isothiocyanate, 2-furylmethyl isothiocyanate, phenyl isothiocyanate, 2-methylphenyl isothiocyanate, 3-methylphenyl isothiocyanate, p-tolyl isothiocyanate, 2-ethylphenyl isothiocyanate, benzyl isothiocyanate, 2-phenylethyl isothiocyanate, alpha-methylbenzyl isothiocyanate, 3-phenylpropyl isothiocyanate, 2-isopropylphenyl isothiocyanate, 4-isopropylphenyl isothiocyanate, 4-butylphenyl isothiocyanate, 4-pentafluorosulphanyl isothiocyanate, 2-fluorophenyl isothiocyanate, 3-fluorophenyl isothiocyanate, 4-fluorophenyl isothiocyanate, 2-chlorophenyl isothiocyanate, 3-chlorophenyl isothiocyanate, 4-chlorophenyl isothiocyanate, 2-bromophenyl isothiocyanate, 3-bromophenyl isothiocyanate, 4-bromophenyl isothiocyanate, 4-pentafluorosulphanyl isothiocyanate, 2-iodophenyl isothiocyanate, 4-iodophenyl isothiocyanate, 4-fluorobenzyl isothiocyanate, 1-(4-fluorophenyl)ethyl isothiocyanate, 2-chlorobenzyl isothiocyanate, 4-chlorobenzyl isothiocyanate, 2-(4-chlorophenyl)ethyl isothiocyanate, 2-(trifluoromethyl)phenyl isothiocyanate, 3-(trifluoromethyl)phenyl isothiocyanate, 4-(trifluoromethyl)phenyl isothiocyanate, 2-(methylthio)phenyl isothiocyanate, 3-(methylthio)phenyl isothiocyanate, 4-(methylthio)phenyl isothiocyanate, 2-methoxyphenyl isothiocyanate, 3-methoxyphenyl isothiocyanate, 4-methoxyphenyl isothiocyanate, 4-methoxybenzyl isothiocyanate, 2-nitrophenyl isothiocyanate, 3-nitrophenyl isothiocyanate, 4-nitrophenyl isothiocyanate, 3-pyridyl isothiocyanate, 4-cyanophenyl isothiocyanate, 3-cyanophenyl isothiocyanate, (4-isothiocyanato phenyl)dimethylamine, 4-diethylaminophenyl isothiocyanate, 4-acetylphenyl isothiocyanate, 3-carbonylphenyl isothiocyanate, 4-ethoxyphenyl isothiocyanate, 4-isothiocyanatophenylacetate, 4-benzyloxyphenyl isothiocyanate, 2-methoxycarbonylphenyl isothiocyanate, 3-methoxycarbonylphenyl isothiocyanate, 4-methoxycarbonylphenyl isothiocyanate, ethyl-2-isothiocyanatobenzoate, 4-ethoxycarbonylphenyl isothiocyanate, 2,4-dimethylphenyl isothiocyanate, 2,6-dimethylphenyl isothiocyanate, 3,5-dimethylphenyl isothiocyanate, 2-ethyl-6-methylphenyl isothiocyanate, 2-ethyl-6-isopropylphenyl isothiocyanate, 2,6-diethylphenyl isothiocyanate, 2,6-diisopropylphenyl isothiocyanate, 2-chloro-6-methylphenyl isothiocyanate, 5-chloro-2-methylphenyl isothiocyanate, 3-chloro-4-methylphenyl isothiocyanate, 4-chloro-2-methylphenyl isothiocyanate, 4-bromo-2-methylphenyl isothiocyanate, 2-bromo-4-methylphenyl isothiocyanate, 2,4-difluorophenyl isothiocyanate, 2,6-difluorophenyl isothiocyanate, 2,5-difluorophenyl isothiocyanate, 2,3-dichlorophenyl isothiocyanate, 2,6-dichlorophenyl isothiocyanate, 2,5-dichlorophenyl isothiocyanate, 3,4-dichlorophenyl isothiocyanate, 2,4-dichlorophenyl isothiocyanate, 3,5-dichlorophenyl isothiocyanate, 3,4-dichlorobenzyl isothiocyanate, 4-bromo-2-chlorophenyl isothiocyanate, 4-chloro-3-nitrophenyl isothiocyanate, 2-chloro-4-nitrophenyl isothiocyanate, 5-chloro-2-methoxyphenyl isothiocyanate, 2-chloro-5-(trifluoromethyl)phenyl isothiocyanate, 4-chloro-3-(trifluoromethyl)phenyl isothiocyanate, 4-bromo-2-(trifluoromethyl) isothiocyanate, 3,5-bis-(trifluoromethyl)phenyl isothiocyanate, 2-methoxy-5-methylphenyl isothiocyanate, 2,5-dimethoxyphenyl isothiocyanate, 2,4-dimethoxyphenyl isothiocyanate, 3,5-dimethoxyphenyl isothiocyanate, 3,4-dimethoxyphenyl isothiocyanate, 4-methoxy-2-nitrophenyl isothiocyanate, 2-methoxy-4-nitrophenyl isothiocyanate, 4-methyl-2-nitrophenyl isothiocyanate, (2-methoxy-5-phenyl)phenyl isothiocyanate, 2,4,6-trifluorophenyl isothiocyanate, 2,4,5-trichlorophenyl isothiocyanate, 2,4,6-trichlorophenyl isothiocyanate, 2,3,4-trichlorophenyl isothiocyanate, 2,4,6-tribromphenyl isothiocyanate, 2,4,6-trimethylphenyl isothiocyanate, 4-bromo-2,6-dimethylphenyl isothiocyanate, 3,4,5-trimethoxyphenyl isothiocyanate, 2,3,5,6-tetrafluorophenyl isothiocyanate, 2,3,4,5-tetrachlorophenyl isothiocyanate, pentafluorophenyl isothiocyanate, 1-naphthyl isothiocyanate, 3,4-methylendioxobenzyl isothiocyanate, triphenylmethyl isothiocyanate, 4-(trans-4-propylcyclohexyl)phenyl isothiocyanate, 4-tert-butylphenylisothiocyanat.

The following substituted spiro compounds according to the invention were prepared as described under 3b.

| | Name | [M + H] |
|---|---|---|
| 1 | 3-[4-(3-chloropyridin-2-yl)piperazine-1-carbonyl]-1-oxa-2,8-diazaspiro[4.5]dec-2-ene-8-carboxylic acid-(2- | 502.0 |
| | fluorophenyl)amide | |
| 2 | 1-oxa-2,8-diazaspiro[4.5]dec-2-ene-3,8-dicarboxylic acid-3-[(5-chloropyridin-2-yl)amide]-8-[(2-fluorophenyl)amide] | 432.9 |
| 3 | 1-oxa-2,8-diazaspiro[4.5]dec-2-ene-3,8-dicarboxylic acid-3-(4-chlorobenzylamide)-8-[(3-fluorophenyl)amide] | 445.9 |
| 4 | 3-[4-(3-chloropyridin-2-yl)piperazine-1-carbonyl]-1-oxa-2,8-diazaspiro[4.5]dec-2-ene-8-carboxylic acid-(3- | 502.0 |
| | fluorophenyl)amide | |
| 5 | 1-oxa-2,8-diazaspiro[4.5]dec-2-ene-3,8-dicarboxylic acid-3-[(5-chloropyridin-2-yl)amide]-8-[(3-fluorophenyl)amide] | 432.9 |
| 6 | 1-oxa-2,8-diazaspiro[4.5]dec-2-ene-3,8-dicarboxylic acid-8-[(2-chlorophenyl)amide]-3-(3,4-dimethoxybenzylamide) | 488.0 |
| 7 | 1-oxa-2,8-diazaspiro[4.5]dec-2-ene-3,8-dicarboxylic acid-3-(4-chlorobenzylamide)-8-[(2-chlorophenyl)amide] | 462.3 |
| 8 | 3-[4-(3-chloropyridin-2-yl)piperazine-1-carbonyl]-1-oxa-2,8-diazaspiro[4.5]dec-2-ene-8-carboxylic acid-(2- | 518.4 |
| | chlorophenyl)amide | |
| 9 | 1-oxa-2,8-diazaspiro[4.5]dec-2-ene-3,8-dicarboxylic acid-8-[(2-chlorophenyl)amide]-3-[(5-chloropyridin-2-yl)amide] | 449.3 |
| 10 | 1-oxa-2,8-diazaspiro[4.5]dec-2-ene-3,8-dicarboxylic acid-8-[(3-bromophenyl)amide]-3-(3,4-dimethoxybenzylamide) | 532.4 |
| 11 | 3-[4-(3-chloropyridin-2-yl)piperazine-1-carbonyl]-1-oxa-2,8-diazaspiro[4.5]dec-2-ene-8-carboxylic acid-(3- | 562.9 |
| | bromophenyl)amide | |
| 12 | 1-oxa-2,8-diazaspiro[4.5]dec-2-ene-3,8-dicarboxylic acid-8-[(3-bromophenyl)amide]-3-[(5-chloropyridin-2-yl)amide] | 493.8 |
| 13 | 1-oxa-2,8-diazaspiro[4.5]dec-2-ene-3,8-dicarboxylic acid-3-(3,4-dimethoxybenzylamide)-8-[(2-methoxyphenyl)amide] | 483.5 |
| 14 | 1-oxa-2,8-diazaspiro[4.5]dec-2-ene-3,8-dicarboxylic acid-3-(4-chlorobenzylamide)-8-[(2-methoxyphenyl)amide] | 457.9 |
| 15 | 3-[4-(3-chloropyridin-2-yl)piperazine-1-carbonyl]-1-oxa-2,8-diazaspiro[4.5]dec-2-ene-8-carboxylic acid-(2- | 514.0 |
| | methoxyphenyl)amide | |
| 16 | 1-oxa-2,8-diazaspiro[4.5]dec-2-ene-3,8-dicarboxylic acid-3-(3,4-dimethoxybenzylamide)-8-[(3-methoxyphenyl)amide] | 483.5 |
| 17 | 3-[4-(3-chloropyridin-2-yl)piperazine-1-carbonyl]-1-oxa-2,8-diazaspiro[4.5]dec-2-ene-8-carboxylic acid-(3- | 514.0 |
| | methoxyphenyl)amide | |
| 18 | 1-oxa-2,8-diazaspiro[4.5]dec-2-ene-3,8-dicarboxylic acid-3-[(5-chloropyridin-2-yl)amide]-8-[(3-methoxyphenyl)amide] | 444.9 |
| 19 | 3-[4-(3-chloropyridin-2-yl)piperazine-1-carbonyl]-1-oxa-2,8-diazaspiro[4.5]dec-2-ene-8-carboxylic acid-(4- | 576.1 |
| | phenoxyphenyl)amide | |
| 20 | 1-oxa-2,8-diazaspiro[4.5]dec-2-ene-3,8-dicarboxylic acid-8-[(2-chloro-5-trifluoromethylphenyl)amide]-3-(3,4- | 556.0 |
| | dimethoxybenzylamide) | |
| 21 | 1-oxa-2,8-diazaspiro[4.5]dec-2-ene-3,8-dicarboxylic acid-3-(4-chlorobenzylamide)-8-[(2-chloro-5- | 530.3 |
| | trifluoromethylphenyl)amide] | |
| 22 | 3-[4-(3-chloropyridin-2-yl)piperazine-1-carbonyl]-1-oxa-2,8-diazaspiro[4.5]dec-2-ene-8-carboxylic acid-(2-chloro-5- | 586.4 |
| | trifluoromethylphenyl)amide | |
| 23 | 1-oxa-2,8-diazaspiro[4.5]dec-2-ene-3,8-dicarboxylic acid-3-[(5-chloropyridin-2-yl)amide]-8-[(2-chloro-5- | 517.3 |
| | trifluoromethylphenyl)amide] | |
| 24 | 1-oxa-2,8-diazaspiro[4.5]dec-2-ene-3,8-dicarboxylic acid-8-[(4-chloro-2-trifluoromethylphenyl)amide]-3-(3,4- | 556.0 |
| | dimethoxybenzylamide) | |
| 25 | 3-[4-(3-chloropyridin-2-yl)piperazine-1-carbonyl]-1-oxa-2,8-diazaspiro[4.5]dec-2-ene-8-carboxylic acid-(4-chloro-2- | 586.4 |
| | trifluoromethylphenyl)amide | |
| 26 | 1-oxa-2,8-diazaspiro[4.5]dec-2-ene-3,8-dicarboxylic acid-3-[(5-chloropyridin-2-yl)amide]-8-[(4-chloro-2- | 517.3 |
| | trifluoromethylphenyl)amide] | |
| 27 | 1-oxa-2,8-diazaspiro[4.5]dec-2-ene-3,8-dicarboxylic acid-8-[(4-chloro-3-trifluoromethylphenyl)amide]-3-(3,4- | 556.0 |
| | dimethoxybenzylamide) | |
| 28 | 3-[4-(3-chloropyridin-2-yl)piperazine-1-carbonyl]-1-oxa-2,8-diazaspiro[4.5]dec-2-ene-8-carboxylic acid-(4-chloro-3- | 586.4 |
| | trifluoromethylphenyl)amide | |
| 29 | 1-oxa-2,8-diazaspiro[4.5]dec-2-ene-3,8-dicarboxylic acid-3-[(5-chloropyridin-2-yl)amide]-8-[(4-chloro-3- | 517.3 |
| | trifluoromethylphenyl)amide] | |
| 30 | 1-oxa-2,8-diazaspiro[4.5]dec-2-ene-3,8-dicarboxylic acid-3-[(2-tert-butyl-6-methylphenyl)amide]-8-(4- | 498.0 |
| | chlorobenzylamide) | |
| 31 | 3-[4-(3-chloropyridin-2-yl)piperazine-1-carbonyl]-1-oxa-2,8-diazaspiro[4.5]dec-2-ene-8-carboxylic acid-(2-tert-butyl-6- | 554.1 |
| | methylphenyl)amide | |
| 32 | 1-oxa-2,8-diazaspiro[4.5]dec-2-ene-3,8-dicarboxylic acid-3-(3,4-dimethoxybenzylamide)-8-[(4- | 537.5 |
| | trifluoromethoxyphenyl)amide] | |
| 33 | 1-oxa-2,8-diazaspiro[4.5]dec-2-ene-3,8-dicarboxylic acid-3-(4-chlorobenzylamide)-8-[(4-trifluoromethoxyphenyl)amide] | 511.9 |
| 34 | 3-[4-(3-chloropyridin-2-yl)piperazine-1-carbonyl]-1-oxa-2,8-diazaspiro[4.5]dec-2-ene-8-carboxylic acid-(4- | 568.0 |
| | trifluoromethoxyphenyl)amide | |
| 35 | 1-oxa-2,8-diazaspiro[4.5]dec-2-ene-3,8-dicarboxylic acid-3-[(5-chloropyridin-2-yl)amide]-8-[(4- | 498.9 |
| | trifluoromethoxyphenyl)amide] | |
| 36 | 1-oxa-2,8-diazaspiro[4.5]dec-2-ene-3,8-dicarboxylic acid-3-(3,4-dimethoxybenzylamide)-8-(phenethylamide) | 481.6 |
| 37 | 1-oxa-2,8-diazaspiro[4.5]dec-2-ene-3,8-dicarboxylic acid-3-(4-chlorobenzylamide)-8-(phenethylamide) | 456.0 |
| 38 | 3-[4-(3-chloropyridin-2-yl)piperazine-1-carbonyl]-1-oxa-2,8-diazaspiro[4.5]dec-2-ene-8-carboxylic acid phenethylamide | 512.0 |
| 39 | 1-oxa-2,8-diazaspiro[4.5]dec-2-ene-3,8-dicarboxylic acid-3-(3,4-dimethoxybenzylamide)-8-phenylamide | 453.5 |
| 40 | 3-[4-(3-chloropyridin-2-yl)piperazine-1-carbonyl]-1-oxa-2,8-diazaspiro[4.5]dec-2-ene-8-carboxylic acid phenylamide | 484.0 |
| 41 | 1-oxa-2,8-diazaspiro[4.5]dec-2-ene-3,8-dicarboxylic acid-3-[(5-chloropyridin-2-yl)amide]-8-phenylamide | 414.9 |
| 42 | 1-oxa-2,8-diazaspiro[4.5]dec-2-ene-3,8-dicarboxylic acid-3-(4-chlorobenzylamide)-8-m-tolylamide | 441.9 |
| 43 | 3-[4-(3-chloropyridin-2-yl)piperazine-1-carbonyl]-1-oxa-2,8-diazaspiro[4.5]dec-2-ene-8-carboxylic acid-m-tolylamide | 498.0 |
| 44 | 1-oxa-2,8-diazaspiro[4.5]dec-2-ene-3,8-dicarboxylic acid-3-(3,4-dimethoxybenzylamide)-8-[(4-fluorophenyl)amide] | 471.5 |
| 45 | 3-[4-(3-chloropyridin-2-yl)piperazine-1-carbonyl]-1-oxa-2,8-diazaspiro[4.5]dec-2-ene-8-carboxylic acid-(4- | 502.0 |
| | fluorophenyl)amide | |
| 46 | 1-oxa-2,8-diazaspiro[4.5]dec-2-ene-3,8-dicarboxylic acid-8-[(3-chlorophenyl)amide]-3-(3,4-dimethoxybenzylamide) | 488.0 |
| 47 | 3-[4-(3-chloropyridin-2-yl)piperazine-1-carbonyl]-1-oxa-2,8-diazaspiro[4.5]dec-2-ene-8-carboxylic acid-(3- | 518.4 |
| | chlorophenyl)amide | |
| 48 | 1-oxa-2,8-diazaspiro[4.5]dec-2-ene-3,8-dicarboxylic acid-8-[(3-chlorophenyl)amide]-3-[(5-chloropyridin-2-yl)amide] | 449.3 |
| 49 | 1-oxa-2,8-diazaspiro[4.5]dec-2-ene-3,8-dicarboxylic acid-8-[(4-chlorophenyl)amide]-3-(3,4-dimethoxybenzylamide) | 488.0 |
| 50 | 1-oxa-2,8-diazaspiro[4.5]dec-2-ene-3,8-dicarboxylic acid-3-(4-chlorobenzylamide)-8-[(4-chlorophenyl)amide] | 462.3 |
| 51 | 3-[4-(3-chloropyridin-2-yl)piperazine-1-carbonyl]-1-oxa-2,8-diazaspiro[4.5]dec-2-ene-8-carboxylic acid-(4- | 518.4 |
| | chlorophenyl)amide | |
| 52 | 3-[4-(3-chloropyridin-2-yl)piperazine-1-carbonyl]-1-oxa-2,8-diazaspiro[4.5]dec-2-ene-8-carboxylic acid-(4- | 514.0 |
| | methoxyphenyl)amide | |
| 53 | 1-oxa-2,8-diazaspiro[4.5]dec-2-ene-3,8-dicarboxylic acid-3-(3,4-dimethoxybenzylamide)-8-[(2- | 499.6 |
| | methylsulphanylphenyl)amide] | |
| 54 | 3-[4-(3-chloropyridin-2-yl)piperazine-1-carbonyl]-1-oxa-2,8-diazaspiro[4.5]dec-2-ene-8-carboxylic acid-(2- | 530.1 |
| | methylsulphanylphenyl)amide | |
| 55 | 1-oxa-2,8-diazaspiro[4.5]dec-2-ene-3,8-dicarboxylic acid-3-(3,4-dimethoxybenzylamide)-8-[(3- | 499.6 |
| | methylsulphanylphenyl)amide] | |
| 56 | 3-[4-(3-chloropyridin-2-yl)piperazine-1-carbonyl]-1-oxa-2,8-diazaspiro[4.5]dec-2-ene-8-carboxylic acid-(3- | 530.1 |
| | methylsulphanylphenyl)amide | |
| 57 | 1-oxa-2,8-diazaspiro[4.5]dec-2-ene-3,8-dicarboxylic acid-3-(3,4-dimethoxybenzylamide)-8-[(4- | 499.6 |
| | methylsulphanylphenyl)amide] | |
| 58 | 3-[4-(3-chloropyridin-2-yl)piperazine-1-carbonyl]-1-oxa-2,8-diazaspiro[4.5]dec-2-ene-8-carboxylic acid-(4- | 530.1 |
| | methylsulphanylphenyl)amide | |
| 59 | 1-oxa-2,8-diazaspiro[4.5]dec-2-ene-3,8-dicarboxylic acid-3-[(5-chloropyridin-2-yl)amide]-8-[(4- | 461.0 |
| | methylsulphanylphenyl)amide] | |
| 60 | 1-oxa-2,8-diazaspiro[4.5]dec-2-ene-3,8-dicarboxylic acid-3-(3,4-dimethoxybenzylamide)-8-[(2-isopropylphenyl)amide] | 495.6 |
| 61 | 3-[4-(3-chloropyridin-2-yl)piperazine-1-carbonyl]-1-oxa-2,8-diazaspiro[4.5]dec-2-ene-8-carboxylic acid-(2- | 526.1 |
| | isopropylphenyl)amide | |
| 62 | 1-oxa-2,8-diazaspiro[4.5]dec-2-ene-3,8-dicarboxylic acid-3-[(5-chloropyridin-2-yl)amide]-8-[(2-isopropylphenyl)amide] | 456.9 |
| 63 | 3-[4-(3-chloropyridin-2-yl)piperazine-1-carbonyl]-1-oxa-2,8-diazaspiro[4.5]dec-2-ene-8-carboxylic acid-(4- | 526.1 |
| | isopropylphenyl)amide | |
| 64 | 1-oxa-2,8-diazaspiro[4.5]dec-2-ene-3,8-dicarboxylic acid-3-(3,4-dimethoxybenzylamide)-8-[(2- | 521.5 |
| | trifluoromethylphenyl)amide] | |
| 65 | 3-[4-(3-chloropyridin-2-yl)piperazine-1-carbonyl]-1-oxa-2,8-diazaspiro[4.5]dec-2-ene-8-carboxylic acid-(2- | 552.0 |
| | trifluoromethylphenyl)amide | |
| 66 | 1-oxa-2,8-diazaspiro[4.5]dec-2-ene-3,8-dicarboxylic acid-3-[(5-chloropyridin-2-yl)amide]-8-[(2- | 482.9 |
| | trifluoromethylphenyl)amide] | |
| 67 | 1-oxa-2,8-diazaspiro[4.5]dec-2-ene-3,8-dicarboxylic acid-3-(3,4-dimethoxybenzylamide)-8-[(3- | 521.5 |
| | trifluoromethylphenyl)amide] | |
| 68 | 3-[4-(3-chloropyridin-2-yl)piperazine-1-carbonyl]-1-oxa-2,8-diazaspiro[4.5]dec-2-ene-8-carboxylic acid-(3- | 552.0 |
| | trifluoromethylphenyl)amide | |
| 69 | 1-oxa-2,8-diazaspiro[4.5]dec-2-ene-3,8-dicarboxylic acid-3-[(5-chloropyridin-2-yl)amide]-8-[(3- | 482.9 |
| | trifluoromethylphenyl)amide] | |
| 70 | 1-oxa-2,8-diazaspiro[4.5]dec-2-ene-3,8-dicarboxylic acid-3-(3,4-dimethoxybenzylamide)-8-[(4- | 521.5 |
| | trifluoromethylphenyl)amide] | |
| 71 | 3-[4-(3-chloropyridin-2-yl)piperazine-1-carbonyl]-1-oxa-2,8-diazaspiro[4.5]dec-2-ene-8-carboxylic acid-(4- | 552.0 |
| | trifluoromethylphenyl)amide | |
| 72 | 1-oxa-2,8-diazaspiro[4.5]dec-2-ene-3,8-dicarboxylic acid-8-cyclohexylamide-3-(3,4-dimethoxybenzylamide) | 459.6 |
| 73 | 1-oxa-2,8-diazaspiro[4.5]dec-2-ene-3,8-dicarboxylic acid-3-(4-chlorobenzylamide)-8-cyclohexylamide | 434.0 |
| 74 | 1-oxa-2,8-diazaspiro[4.5]dec-2-ene-3,8-dicarboxylic acid-8-benzylamide-3-(3,4-dimethoxybenzylamide) | 467.5 |
| 75 | 1-oxa-2,8-diazaspiro[4.5]dec-2-ene-3,8-dicarboxylic acid-3-(3,4-dimethoxybenzylamide)-8-o-tolylamide | 467.5 |
| 76 | 1-oxa-2,8-diazaspiro[4.5]dec-2-ene-3,8-dicarboxylic acid-3-(4-chlorobenzylamide)-8-o-tolylamide | 441.9 |
| 77 | 1-oxa-2,8-diazaspiro[4.5]dec-2-ene-3,8-dicarboxylic acid-3-[(5-chloropyridin-2-yl)amide]-8-o-tolylamide | 428.9 |
| 78 | 1-oxa-2,8-diazaspiro[4.5]dec-2-ene-3,8-dicarboxylic acid-3-(3,4-dimethoxybenzylamide)-8-[(2-ethylphenyl)amide] | 481.6 |
| 79 | 3-[4-(3-chloropyridin-2-yl)piperazine-1-carbonyl]-1-oxa-2,8-diazaspiro[4.5]dec-2-ene-8-carboxylic acid-(2- | 512.0 |
| | ethylphenyl)amide | |
| 80 | 1-oxa-2,8-diazaspiro[4.5]dec-2-ene-3,8-dicarboxylic acid-3-[(5-chloropyridin-2-yl)amide]-8-[(2-ethylphenyl)amide] | |
| 81 | 1-oxa-2,8-diazaspiro[4.5]dec-2-ene-3,8-dicarboxylic acid-3-(4-chlorobenzylamide)-8-[(4-ethylphenyl)amide] | 456.0 |
| 82 | 1-oxa-2,8-diazaspiro[4.5]dec-2-ene-3,8-dicarboxylic acid-3-[(5-chloropyridin-2-yl)amide]-8-[(4-ethylphenyl)amide] | 442.9 |
| 83 | 1-oxa-2,8-diazaspiro[4.5]dec-2-ene-3,8-dicarboxylic acid-3-(3,4-dimethoxybenzylamide)-8-[(2-fluorophenyl)amide] | 471.5 |
| 84 | 1-oxa-2,8-diazaspiro[4.5]dec-2-ene-3,8-dicarboxylic acid-3-(3,4-dimethoxybenzylamide)-8-p-tolylamide | 467.5 |
| 85 | 1-oxa-2,8-diazaspiro[4.5]dec-2-ene-3,8-dicarboxylic acid-3-(4-chlorobenzylamide)-8-p-tolylamide | 441.9 |
| 86 | 3-[4-(3-chloropyridin-2-yl)piperazine-1-carbonyl]-1-oxa-2,8-diazaspiro[4.5]dec-2-ene-8-carboxylic acid p-tolylamide | 498.0 |
| 87 | 1-oxa-2,8-diazaspiro[4.5]dec-2-ene-3,8-dicarboxylic acid-3-[(5-chloropyridin-2-yl)amide]-8-p-tolylamide | 428.9 |
| 88 | 1-oxa-2,8-diazaspiro[4.5]dec-2-ene-3,8-dicarboxylic acid-3-(4-chlorobenzylamide)-8-[(3-ethylphenyl)amide] | 456.0 |
| 89 | 3-[4-(3-chloropyridin-2-yl)piperazine-1-carbonyl]-1-oxa-2,8-diazaspiro[4.5]dec-2-ene-8-carboxylic acid-(3- | 512.0 |
| | ethylphenyl)amide | |
| 90 | 1-oxa-2,8-diazaspiro[4.5]dec-2-ene-3,8-dicarboxylic acid-3-[(5-chloropyridin-2-yl)amide]-8-[(3-ethylphenyl)amide] | 442.9 |
| 91 | 1-oxa-2,8-diazaspiro[4.5]dec-2-ene-3,8-dicarboxylic acid-3-(3,4-dimethoxybenzylamide)-8-[(2-propylphenyl)amide] | 495.6 |
| 92 | 1-oxa-2,8-diazaspiro[4.5]dec-2-ene-3,8-dicarboxylic acid-3-(4-chlorobenzylamide)-8-[(2-propylphenyl)amide] | 470.0 |
| 93 | 1-oxa-2,8-diazaspiro[4.5]dec-2-ene-3,8-dicarboxylic acid-3-(4-chlorobenzylamide)-8-[(2-propylphenyl)amide] | 470.0 |
| 94 | 3-[4-(3-chloropyridin-2-yl)piperazine-1-carbonyl]-1-oxa-2,8-diazaspiro[4.5]dec-2-ene-8-carboxylic acid-(2- | 526.1 |
| | propylphenyl)amide | |
| 95 | 1-oxa-2,8-diazaspiro[4.5]dec-2-ene-3,8-dicarboxylic acid-3-[(5-chloropyridin-2-yl)amide]-8-[(2-propylphenyl)amide] | 456.9 |
| 96 | 1-oxa-2,8-diazaspiro[4.5]dec-2-ene-3,8-dicarboxylic acid-8-[(2-bromophenyl)amide]-3-(3,4-dimethoxybenzylamide) | 532.4 |
| 97 | 1-oxa-2,8-diazaspiro[4.5]dec-2-ene-3,8-dicarboxylic acid-8-[(2-bromophenyl)amide]-3-(4-chlorobenzylamide) | 506.8 |
| 98 | 3-[4-(3-chloropyridin-2-yl)piperazine-1-carbonyl]-1-oxa-2,8-diazaspiro[4.5]dec-2-ene-8-carboxylic acid-(2- | 562.9 |
| | bromophenyl)amide | |
| 99 | 3-[4-(3-chloropyridin-2-yl)piperazine-1-carbonyl]-1-oxa-2,8-diazaspiro[4.5]dec-2-ene-8-carboxylic acid-(4- | 562.9 |
| | bromophenyl)amide | |
| 100 | 3-[4-(3-chloropyridin-2-yl)piperazine-1-carbonyl]-1-oxa-2,8-diazaspiro[4.5]dec-2-ene-8-carboxylic acid-biphenyl-4- | 560.1 |
| | ylamide | |
| 101 | 1-oxa-2,8-diazaspiro[4.5]dec-2-ene-3,8-dicarboxylic acid-3-(3,4-dimethoxybenzylamide)-8-[(2-phenoxyphenyl)amide] | 545.6 |
| 102 | 3-[4-(3-chloropyridin-2-yl)piperazine-1-carbonyl]-1-oxa-2,8-diazaspiro[4.5]dec-2-ene-8-carboxylic acid-(2- | 576.1 |
| | phenoxyphenyl)amide | |
| 103 | 1-oxa-2,8-diazaspiro[4.5]dec-2-ene-3,8-dicarboxylic acid-3-[(5-chloropyridin-2-yl)amide]-8-[(2-phenoxyphenyl)amide] | 507.0 |
| 104 | 1-oxa-2,8-diazaspiro[4.5]dec-2-ene-3,8-dicarboxylic acid-3-(4-chlorobenzylamide)-8-[(2-trifluoromethoxyphenyl)amide] | 511.9 |
| 105 | 3-[4-(3-chloropyridin-2-yl)piperazine-1-carbonyl]-1-oxa-2,8-diazaspiro[4.5]dec-2-ene-8-carboxylic acid-(2- | 568.0 |
| | trifluoromethoxyphenyl)amide | |
| 106 | 1-oxa-2,8-diazaspiro[4.5]dec-2-ene-3,8-dicarboxylic acid-3-[(5-chloropyridin-2-yl)amide]-8-[(2- | 498.9 |
| | trifluoromethoxyphenyl)amide] | |
| 107 | 1-oxa-2,8-diazaspiro[4.5]dec-2-ene-3,8-dicarboxylic acid-3-(3,4-dimethoxybenzylamide)-8-(4-methylbenzylamide) | 481.6 |
| 108 | 1-oxa-2,8-diazaspiro[4.5]dec-2-ene-3,8-dicarboxylic acid-3-(4-chlorobenzylamide)-8-(4-methylbenzylamide) | 456.0 |
| 109 | 3-[4-(3-chloropyridin-2-yl)piperazine-1-carbonyl]-1-oxa-2,8-diazaspiro[4.5]dec-2-ene-8-carboxylic acid-4- | 512.0 |
| | methylbenzylamide | |
| 110 | 1-oxa-2,8-diazaspiro[4.5]dec-2-ene-3,8-dicarboxylic acid-3-[(5-chloropyridin-2-yl)amide]-8-(4-methylbenzylamide) | 442.9 |
| 111 | 1-oxa-2,8-diazaspiro[4.5]dec-2-ene-3,8-dicarboxylic acid-3-(3,4-dimethoxybenzylamide)-8-(4-methoxybenzylamide) | 497.6 |
| 112 | 1-oxa-2,8-diazaspiro[4.5]dec-2-ene-3,8-dicarboxylic acid-3-(4-chlorobenzylamide)-8-(4-methoxybenzylamide) | 472.0 |
| 113 | 3-[4-(3-chloropyridin-2-yl)piperazine-1-carbonyl]-1-oxa-2,8-diazaspiro[4.5]dec-2-ene-8-carboxylic acid-4- | 528.0 |
| | methoxybenzylamide | |
| 114 | 1-oxa-2,8-diazaspiro[4.5]dec-2-ene-3,8-dicarboxylic acid-8-[(4-tert-butylphenyl)amide]-3-(3,4-dimethoxybenzylamide) | 509.6 |
| 115 | 1-oxa-2,8-diazaspiro[4.5]dec-2-ene-3,8-dicarboxylic acid-8-[(4-tert-butylphenyl)amide]-3-(4-chlorobenzylamide) | 484.0 |
| 116 | 3-[4-(3-chloropyridin-2-yl)piperazine-1-carbonyl]-1-oxa-2,8-diazaspiro[4.5]dec-2-ene-8-carboxylic acid-(4-tert- | 540.1 |
| | butylphenyl)amide | |
| 117 | 1-oxa-2,8-diazaspiro[4.5]dec-2-ene-3,8-dicarboxylic acid-8-[(4-tert-butylphenyl)amide]-3-[(5-chloropyridin-2-yl)amide] | 471.0 |
| 118 | 8-(2-methoxyphenylthiocarbamoyl)-1-oxa-2,8-diazaspiro[4.5]dec-2-ene-3-carboxylic acid-3,4-dimethoxybenzylamide | 499.6 |
| 119 | 3-[4-(3-chloropyridin-2-yl)piperazine-1-carbonyl]-1-oxa-2,8-diazaspiro[4.5]dec-2-ene-8-thiocarboxylic acid-(2- | 530.1 |
| | methoxyphenyl)amide | |
| 120 | 8-(cyclohexylmethylthiocarbamoyl)-1-oxa-2,8-diazaspiro[4.5]dec-2-ene-3-carboxylic acid-3,4-dimethoxybenzylamide | 489.7 |
| 121 | 8-(cyclohexylmethylthiocarbamoyl)-1-oxa-2,8-diazaspiro[4.5]dec-2-ene-3-carboxylic acid-4-chlorobenzylamide | 464.0 |
| 122 | 3-[4-(3-chloropyridin-2-yl)piperazine-1-carbonyl]-1-oxa-2,8-diazaspiro[4.5]dec-2-ene-8-thiocarboxylic acid | 520.1 |
| | cyclohexylmethylamide | |
| 123 | 8-cyclooctylthiocarbamoyl-1-oxa-2,8-diazaspiro[4.5]dec-2-ene-3-carboxylic acid-3,4-dimethoxybenzylamide | 503.7 |
| 124 | 8-cyclooctylthiocarbamoyl-1-oxa-2,8-diazaspiro[4.5]dec-2-ene-3-carboxylic acid-4-chlorobenzylamide | 478.1 |
| 125 | 8-(3-morpholin-4-yl-propylthiocarbamoyl)-1-oxa-2,8-diazaspiro[4.5]dec-2-ene-3-carboxylic acid-4-chlorobenzylamide | 495.1 |
| 126 | 8-(3-morpholin-4-yl-propylthiocarbamoyl)-1-oxa-2,8-diazaspiro[4.5]dec-2-ene-3-carboxylic acid-(5-chloropyridin-2- | 482.0 |
| | yl)amide | |
| 127 | 8-p-tolylthiocarbamoyl-1-oxa-2,8-diazaspiro[4.5]dec-2-ene-3-carboxylic acid-4-chlorobenzylamide | 458.0 |
| 128 | 8-phenethylthiocarbamoyl-1-oxa-2,8-diazaspiro[4.5]dec-2-ene-3-carboxylic acid-3,4-dimethoxybenzylamide | 497.6 |
| 129 | 8-phenethylthiocarbamoyl-1-oxa-2,8-diazaspiro[4.5]dec-2-ene-3-carboxylic acid-4-chlorobenzylamide | 472.0 |
| 130 | 8-(3-phenyl-propylthiocarbamoyl)-1-oxa-2,8-diazaspiro[4.5]dec-2-ene-3-carboxylic acid-(5-chloropyridin-2-yl)amide | 473.0 |
| 131 | 8-(2-fluorophenylthiocarbamoyl)-1-oxa-2,8-diazaspiro[4.5]dec-2-ene-3-carboxylic acid-3,4-dimethoxybenzylamide | 487.6 |
| 132 | 8-(4-fluorophenylthiocarbamoyl)-1-oxa-2,8-diazaspiro[4.5]dec-2-ene-3-carboxylic acid-3,4-dimethoxybenzylamide | 487.6 |
| 133 | 8-(4-fluorophenylthiocarbamoyl)-1-oxa-2,8-diazaspiro[4.5]dec-2-ene-3-carboxylic acid-4-chlorobenzylamide | 462.0 |
| 134 | 8-(2-chlorophenylthiocarbamoyl)-1-oxa-2,8-diazaspiro[4.5]dec-2-ene-3-carboxylic acid-3,4-dimethoxybenzylamide | 504.0 |
| 135 | 8-(2-chlorophenylthiocarbamoyl)-1-oxa-2,8-diazaspiro[4.5]dec-2-ene-3-carboxylic acid-4-chlorobenzylamide | 478.4 |
| 136 | 8-(3-trifluoromethylphenylthiocarbamoyl)-1-oxa-2,8-diazaspiro[4.5]dec-2-ene-3-carboxylic acid-4-chlorobenzylamide | 512.0 |
| 137 | 8-(naphthalen-1-ylthiocarbamoyl)-1-oxa-2,8-diazaspiro[4.5]dec-2-ene-3-carboxylic acid-3,4-dimethoxybenzylamide | 519.6 |
| 138 | 8-(naphthalen-1-ylthiocarbamoyl)-1-oxa-2,8-diazaspiro[4.5]dec-2-ene-3-carboxylic acid-3,4-dimethoxybenzylamide | 519.6 |
| 139 | 8-cyclopentylthiocarbamoyl-1-oxa-2,8-diazaspiro[4.5]dec-2-ene-3-carboxylic acid-3,4-dimethoxybenzylamide | 461.6 |
| 140 | 3-[4-(3-chloropyridin-2-yl)piperazine-1-carbonyl]-1-oxa-2 8-diazaspiro[4.5]dec-2-ene-8-thiocarboxylic acid | 492.1 |
| | cyclopentylamide | |
| 141 | 8-(2-morpholin-4-ylethylthiocarbamoyl)-1-oxa-2,8-diazaspiro[4.5]dec-2-ene-3-carboxylic acid-3,4- | 506.6 |
| | dimethoxybenzylamide | |
| 142 | 8-(2-morpholin-4-ylethylthiocarbamoyl)-1-oxa-2,8-diazaspiro[4.5]dec-2-ene-3-carboxylic acid-4-chlorobenzylamide | 481.0 |
| 143 | 8-(3-chlorophenylthiocarbamoyl)-1-oxa-2,8-diazaspiro[4.5]dec-2-ene-3-carboxylic acid-3,4-dimethoxybenzylamide | 504.0 |
| 144 | 3-[4-(3-chloropyridin-2-yl)piperazine-1-carbonyl]-1-oxa-2,8-diazaspiro[4.5]dec-2-ene-8-thiocarboxylic acid-(4- | 568.0 |
| | trifluoromethylphenyl)amide | |
| 145 | 8-(2-methylsulphanylphenylthiocarbamoyl)-1-oxa-2,8-diazaspiro[4.5]dec-2-ene-3-carboxylic acid-3,4- | 515.7 |
| | dimethoxybenzylamide | |
| 146 | 8-(2-methylsulphanylphenylthiocarbamoyl)-1-oxa-2,8-diazaspiro[4.5]dec-2-ene-3-carboxylic acid-4-chlorobenzylamide | 490.1 |
| 147 | 3-[4-(3-chloropyridin-2-yl)piperazine-1-carbonyl]-1-oxa-2,8-diazaspiro[4.5]dec-2-ene-8-thiocarboxylic acid-(2- | 546.1 |
| | methylsulphanylphenyl)amide | |
| 148 | 8-(4-isopropylphenylthiocarbamoyl)-1-oxa-2,8-diazaspiro[4.5]dec-2-ene-3-carboxylic acid-3,4-dimethoxybenzylamide | 511.7 |
| 149 | 8-(4-isopropylphenylthiocarbamoyl)-1-oxa-2,8-diazaspiro[4.5]dec-2-ene-3-carboxylic acid-4-chlorobenzylamide | 486.0 |
| 150 | 8-(2-iodophenylthiocarbamoyl)-1-oxa-2,8-diazaspiro[4.5]dec-2-ene-3-carboxylic acid-3,4-dimethoxybenzylamide | 595.5 |
| 151 | 8-(2-iodophenylthiocarbamoyl)-1-oxa-2,8-diazaspiro[4.5]dec-2-ene-3-carboxylic acid-4-chlorobenzylamide | 569.9 |
| 152 | 8-(2-trifluoromethylphenylthiocarbamoyl)-1-oxa-2,8-diazaspiro[4.5]dec-2-ene-3-carboxylic acid-3,4- | 537.6 |
| | dimethoxybenzylamide | |
| 153 | 8-(2-trifluoromethylphenylthiocarbamoyl)-1-oxa-2,8-diazaspiro[4.5]dec-2-ene-3-carboxylic acid-4-chlorobenzylamide | 512.0 |
| 154 | 3-[4-(3-chloropyridin-2-yl)piperazine-1-carbonyl]-1-oxa-2,8-diazaspiro[4.5]dec-2-ene-8-thiocarboxylic acid-(2- | 568.0 |
| | trifluoromethylphenyl)amide | |
| 155 | 8-[(benzo[1,3]dioxol-5-ylmethyl)thiocarbamoyl]-1-oxa-2,8-diazaspiro[4.5]dec-2-ene-3-carboxylic acid-3,4- | 527.6 |
| | dimethoxybenzylamide | |
| 156 | 8-[(benzo[1,3]dioxol-5-ylmethyl)thiocarbamoyl]-1-oxa-2,8-diazaspiro[4.5]dec-2-ene-3-carboxylic acid-4- | 502.0 |
| | chlorobenzylamide | |
| 157 | 3-[4-(3-chloropyridin-2-yl)piperazine-1-carbonyl]-1-oxa-2,8-diazaspiro[4.5]dec-2-ene-8-thiocarboxylic acid- | 558.1 |
| | (benzo[1,3]dioxol-5-ylmethyl)amide | |
| 158 | 3-[4-(3-chloropyridin-2-yl)piperazine-1-carbonyl]-1-oxa-2,8-diazaspiro[4.5]dec-2-ene-8-carboxylic acid-(3- | 509.0 |
| | cyanophenyl)amide | |
| 159 | 1-oxa-2,8-diazaspiro[4.5]dec-2-ene-3,8-dicarboxylic acid-3-(3,4-dimethoxybenzylamide)-8-[(2,5- | 513.6 |
| | dimethoxyphenyl)amide] | |
| 160 | 1-oxa-2,8-diazaspiro[4.5]dec-2-ene-3,8-dicarboxylic acid-3-(4-chlorobenzylamide)-8-[(2,5-dimethoxyphenyl)amide] | 488.0 |
| 161 | 3-[4-(3-chloropyridin-2-yl)piperazine-1-carbonyl]-1-oxa-2,8-diazaspiro[4.5]dec-2-ene-8-carboxylic acid-(2,5- | 544.0 |
| | dimethoxyphenyl)amide | |
| 162 | 1-oxa-2,8-diazaspiro[4.5]dec-2-ene-3,8-dicarboxylic acid-3-(3,4-dimethoxybenzylamide)-8-[(2,4- | 481.6 |
| | dimethylphenyl)amide] | |
| 163 | 1-oxa-2,8-diazaspiro[4.5]dec-2-ene-3,8-dicarboxylic acid-3-(4-chlorobenzylamide)-8-[(2,4-dimethylphenyl)amide] | 456.0 |
| 164 | 3-[4-(3-chloropyridin-2-yl)piperazine-1-carbonyl]-1-oxa-2,8-diazaspiro[4.5]dec-2-ene-8-carboxylic acid-(2,4- | 512.0 |
| | dimethylphenyl)amide | |
| 165 | 1-oxa-2,8-diazaspiro[4.5]dec-2-ene-3,8-dicarboxylic acid-8-butylamide-3-(3,4-dimethoxybenzylamide) | 433.5 |
| 166 | 1-oxa-2,8-diazaspiro[4.5]dec-2-ene-3,8-dicarboxylic acid-8-butylamide-3-(4-chlorobenzylamide) | 407.9 |
| 167 | 3-[4-(3-chloropyridin-2-yl)piperazine-1-carbonyl]-1-oxa-2,8-diazaspiro[4.5]dec-2-ene-8-carboxylic acid butylamide | 464.0 |
| 168 | 1-oxa-2,8-diazaspiro[4.5]dec-2-ene-3,8-dicarboxylic acid-3-(3,4-dimethoxybenzylamide)-8-pentylamide | 447.5 |
| 169 | 1-oxa-2,8-diazaspiro[4.5]dec-2-ene-3,8-dicarboxylic acid-3-(4-chlorobenzylamide)-8-pentylamide | 421.9 |
| 170 | 3-[4-(3-chloropyridin-2-yl)piperazine-1-carbonyl]-1-oxa-2,8-diazaspiro[4.5]dec-2-ene-8-carboxylic acid pentylamide | 478.0 |
| 171 | 1-oxa-2,8-diazaspiro[4.5]dec-2-ene-3,8-dicarboxylic acid-3-(3,4-dimethoxybenzylamide)-8-[(2-ethoxyphenyl)amide] | 497.6 |
| 172 | 1-oxa-2,8-diazaspiro[4.5]dec-2-ene-3,8-dicarboxylic acid-3-(4-chlorobenzylamide)-8-[(2-ethoxyphenyl)amide] | 472.0 |
| 173 | 1-oxa-2,8-diazaspiro[4.5]dec-2-ene-3,8-dicarboxylic acid-3-(3,4-dimethoxybenzylamide)-8-[(4-ethoxyphenyl)amide] | 497.6 |
| 174 | 1-oxa-2,8-diazaspiro[4.5]dec-2-ene-3,8-dicarboxylic acid-8-[(2,4-difluorophenyl)amide]-3-(3,4-dimethoxybenzylamide) | 489.5 |
| 175 | 1-oxa-2,8-diazaspiro[4.5]dec-2-ene-3,8-dicarboxylic acid-3-(4-chlorobenzylamide)-8-[(2,4-difluorophenyl)amide] | 463.9 |
| 176 | 1-oxa-2,8-diazaspiro[4.5]dec-2-ene-3,8-dicarboxylic acid-8-[(3-chloro-2-methylphenyl)amide]-3-(3,4- | 502.0 |
| | dimethoxybenzylamide) | |
| 177 | 1-oxa-2,8-diazaspiro[4.5]dec-2-ene-3,8-dicarboxylic acid-3-(4-chlorobenzylamide)-8-[(3-chloro-2-methylphenyl)amide] | 476.4 |
| 178 | 1-oxa-2,8-diazaspiro[4.5]dec-2-ene-3,8-dicarboxylic acid-8-[(4-chloro-2-methylphenyl)amide]-3-(3,4- | 502.0 |
| | dimethoxybenzylamide) | |
| 179 | 1-oxa-2,8-diazaspiro[4.5]dec-2-ene-3,8-dicarboxylic acid-3-(4-chlorobenzylamide)-8-[(4-chloro-2-methylphenyl)amide] | 476.4 |
| 180 | 1-oxa-2,8-diazaspiro[4.5]dec-2-ene-3,8-dicarboxylic acid-8-[(3-chloro-4-methylphenyl)amide]-3-(3,4- | 502.0 |
| | dimethoxybenzylamide) | |
| 181 | 1-oxa-2,8-diazaspiro[4.5]dec-2-ene-3,8-dicarboxylic acid-3-(4-chlorobenzylamide)-8-[(3-chloro-4-methylphenyl)amide] | 476.4 |
| 182 | 1-oxa-2,8-diazaspiro[4.5]dec-2-ene-3,8-dicarboxylic acid-8-[(3-chloro-4-fluorophenyl)amide]-3-(3,4- | 505.9 |
| | dimethoxybenzylamide) | |
| 183 | 1-oxa-2,8-diazaspiro[4.5]dec-2-ene-3,8-dicarboxylic acid-3-(4-chlorobenzylamide)-8-[(3-chloro-4-fluorophenyl)amide] | 480.3 |
| 184 | 1-oxa-2,8-diazaspiro[4.5]dec-2-ene-3,8-dicarboxylic acid-8-[(2,6-diisopropylphenyl)amide]-3-(3,4- | 537.7 |
| | dimethoxybenzylamide) | |
| 185 | 1-oxa-2,8-diazaspiro[4.5]dec-2-ene-3,8-dicarboxylic acid-3-(4-chlorobenzylamide)-8-[(2,6-diisopropylphenyl)amide] | 512.1 |
| 186 | 1-oxa-2,8-diazaspiro[4.5]dec-2-ene-3,8-dicarboxylic acid-8-[(5-chloro-2-methoxyphenyl)amide]-3-(3,4- | 518.0 |
| | dimethoxybenzylamide) | |
| 187 | 1-oxa-2,8-diazaspiro[4.5]dec-2-ene-3,8-dicarboxylic acid-3-(4-chlorobenzylamide)-8-[(5-chloro-2- | 492.4 |
| | methoxyphenyl)amide] | |
| 188 | 1-oxa-2,8-diazaspiro[4.5]dec-2-ene-3,8-dicarboxylic acid-3-(4-chlorobenzylamide)-8-[(3,4,5-trimethoxyphenyl)amide] | 518.0 |
| 189 | 1-oxa-2,8-diazaspiro[4.5]dec-2-ene-3,8-dicarboxylic acid-3-(3,4-dimethoxybenzylamide)-8-[(3,5- | 481.6 |
| | dimethylphenyl)amide] | |
| 190 | 1-oxa-2,8-diazaspiro[4.5]dec-2-ene-3,8-dicarboxylic acid-3-(4-chlorobenzylamide)-8-[(3,5-dimethylphenyl)amide] | 456.0 |
| 191 | 1-oxa-2,8-diazaspiro[4.5]dec-2-ene-3,8-dicarboxylic acid-3-(3,4-dimethoxybenzylamide)-8-[(2,6- | 481.6 |
| | dimethylphenyl)amide] | |
| 192 | 1-oxa-2,8-diazaspiro[4.5]dec-2-ene-3,8-dicarboxylic acid-3-(4-chlorobenzylamide)-8-[(2,6-dimethylphenyl)amide] | 456.0 |
| 193 | 1-oxa-2,8-diazaspiro[4.5]dec-2-ene-3,8-dicarboxylic acid-3-(3,4-dimethoxybenzylamide)-8-[(3,4- | 481.6 |
| | dimethylphenyl)amide] | |
| 194 | 1-oxa-2,8-diazaspiro[4.5]dec-2-ene-3,8-dicarboxylic acid-3-(3,4-dimethoxybenzylamide)-8-[(2,5- | 481.6 |
| | dimethylphenyl)amide] | |
| 195 | 1-oxa-2,8-diazaspiro[4.5]dec-2-ene-3,8-dicarboxylic acid-3-(4-chlorobenzylamide)-8-[(2,5-dimethylphenyl)amide] | 456.0 |
| 196 | 1-oxa-2,8-diazaspiro[4.5]dec-2-ene-3,8-dicarboxylic acid-3-(3,4-dimethoxybenzylamide)-8-[(2-ethyl-6- | 495.6 |
| | methylphenyl)amide] | |
| 197 | 1-oxa-2,8-diazaspiro[4.5]dec-2-ene-3,8-dicarboxylic acid-3-(4-chlorobenzylamide)-8-[(2-ethyl-6-methylphenyl)amide] | 470.0 |
| 198 | 1-oxa-2,8-diazaspiro[4.5]dec-2-ene-3,8-dicarboxylic acid-3-(3,4-dimethoxybenzylamide)-8-[(4-methoxy-2- | 497.6 |
| | methylphenyl)amide] | |
| 199 | 1-oxa-2,8-diazaspiro[4.5]dec-2-ene-3,8-dicarboxylic acid-3-(4-chlorobenzylamide)-8-[(4-methoxy-2- | 472.0 |
| | methylphenyl)amide] | |
| 200 | 1-oxa-2,8-diazaspiro[4.5]dec-2-ene-3,8-dicarboxylic acid-3-(3,4-dimethoxybenzylamide)-8-[(2-methoxy-5- | 497.6 |
| | methylphenyl)amide] | |
| 201 | 1-oxa-2,8-diazaspiro[4.5]dec-2-ene-3,8-dicarboxylic acid-3-(4-chlorobenzylamide)-8-[(2-methoxy-5- | 472.0 |
| | methylphenyl)amide] | |
| 202 | 1-oxa-2,8-diazaspiro[4.5]dec-2-ene-3,8-dicarboxylic acid-3-(3,4-dimethoxybenzylamide)-8-[(2,4,5- | 495.6 |
| | trimethylphenyl)amide] | |
| 203 | 1-oxa-2,8-diazaspiro[4.5]dec-2-ene-3,8-dicarboxylic acid-3-(4-chlorobenzylamide)-8-[(2,4,5-trimethylphenyl)amide] | 470.0 |
| 204 | 1-oxa-2,8-diazaspiro[4.5]dec-2-ene-3,8-dicarboxylic acid-3-(3,4-dimethoxybenzylamide)-8-[(2,4,6- | 495.6 |
| | trimethylphenyl)amide] | |
| 205 | 1-oxa-2,8-diazaspiro[4.5]dec-2-ene-3,8-dicarboxylic acid-3-(4-chlorobenzylamide)-8-[(2,4,6-trimethylphenyl)amide] | 470.0 |
| 206 | 1-oxa-2,8-diazaspiro[4.5]dec-2-ene-3,8-dicarboxylic acid-8-[(2-bromoethyl)amide]-3-(3,4-dimethoxybenzylamide) | 484.4 |
| 207 | 1-oxa-2,8-diazaspiro[4.5]dec-2-ene-3,8-dicarboxylic acid-8-[(2-bromoethyl)amide]-3-(4-chlorobenzylamide) | 458.8 |
| 208 | 1-oxa-2,8-diazaspiro[4.5]dec-2-ene-3,8-dicarboxylic acid-8-[(4-butylphenyl)amide]-3-(3,4-dimethoxybenzylamide) | 509.6 |
| 209 | 1-oxa-2,8-diazaspiro[4.5]dec-2-ene-3,8-dicarboxylic acid-8-[(4-butylphenyl)amide]-3-(4-chlorobenzylamide) | 484.0 |
| 210 | 2-{[3-(4-chlorobenzylcarbamoyl)-1-oxa-2,8-diazaspiro[4.5]dec-2-ene-8-carbonyl]amino}benzoic acid methyl ester | 486.1 |
| 211 | 1-oxa-2,8-diazaspiro[4.5]dec-2-ene-3,8-dicarboxylic acid-8-biphenyl-2-ylamide-3-(3,4-dimethoxybenzylamide) | 529.6 |
| 212 | 1-oxa-2,8-diazaspiro[4.5]dec-2-ene-3,8-dicarboxylic acid-8-biphenyl-2-ylamide-3-(4-chlorobenzylamide) | 504.0 |
| 213 | 1-oxa-2,8-diazaspiro[4.5]dec-2-ene-3,8-dicarboxylic acid-8-[(2,6-dichlorophenyl)amide]-3-(3,4-dimethoxybenzylamide) | 522.4 |
| 214 | 1-oxa-2,8-diazaspiro[4.5]dec-2-ene-3,8-dicarboxylic acid-3-(4-chlorobenzylamide)-8-[(2,6-dichlorophenyl)amide] | 496.8 |
| 215 | 3-{[3-(3,4-dimethoxybenzylcarbamoyl)-1-oxa-2,8-diazaspiro[4.5]dec-2-ene-8-carbonyl]amino}benzoic acid ethyl ester | 525.6 |
| 216 | 3-{[3-(4-chlorobenzylcarbamoyl)-1-oxa-2,8-diazaspiro[4.5]dec-2-ene-8-carbonyl]amino}benzoic acid ethyl ester | 500.0 |
| 217 | 4-{[3-(3,4-dimethoxybenzylcarbamoyl)-1-oxa-2,8-diazaspiro[4.5]dec-2-ene-8-carbonyl]amino}benzoic acid ethyl ester | 525.6 |
| 218 | 4-{[3-(4-chlorobenzylcarbamoyl)-1-oxa-2,8-diazaspiro[4.5]dec-2-ene-8-carbonyl]amino}benzoic acid ethyl ester | 500.0 |
| 219 | 3-[4-(3-chloropyridin-2-yl)piperazine-1-carbonyl]-1-oxa-2,8-diazaspiro[4.5]dec-2-ene-8-carboxylic acid-(2- | 478.0 |
| | fluorophenyl)amide | |
| 220 | 1-oxa-2,8-diazaspiro[4.5]dec-2-ene-3,8-dicarboxylic acid-3-[(5-chloropyridin-2-yl)amide]-8-[(2-fluorophenyl)amide] | 602.5 |
| 221 | 1-oxa-2,8-diazaspiro[4.5]dec-2-ene-3,8-dicarboxylic acid-3-(4-chlorobenzylamide)-8-[(3-fluorophenyl)amide] | 572.0 |
| 222 | 3-[4-(3-chloropyridin-2-yl)piperazine-1-carbonyl]-1-oxa-2,8-diazaspiro[4.5]dec-2-ene-8-carboxylic acid-(3- | 605.6 |
| | fluorophenyl)amide | |
| 223 | 1-oxa-2,8-diazaspiro[4.5]dec-2-ene-3,8-dicarboxylic acid-3-[(5-chloropyridin-2-yl)amide]-8-[(3-fluorophenyl)amide] | 580.0 |
| 224 | 1-oxa-2,8-diazaspiro[4.5]dec-2-ene-3,8-dicarboxylic acid-8-[(2-chlorophenyl)amide]-3-(3,4-dimethoxybenzylamide) | 636.0 |
| 225 | 1-oxa-2,8-diazaspiro[4.5]dec-2-ene-3,8-dicarboxylic acid-3-(4-chlorobenzylamide)-8-[(2-chlorophenyl)amide] | 559.8 |
| 226 | 3-[4-(3-chloropyridin-2-yl)piperazine-1-carbonyl]-1-oxa-2,8-diazaspiro[4.5]dec-2-ene-8-carboxylic acid-(2- | 483.6 |
| | chlorophenyl)amide | |
| 227 | 1-oxa-2,8-diazaspiro[4.5]dec-2-ene-3,8-dicarboxylic acid-8-[(2-chlorophenyl)amide]-3-[(5-chloropyridin-2-yl)amide] | 458.0 |
| 228 | 1-oxa-2,8-diazaspiro[4.5]dec-2-ene-3,8-dicarboxylic acid-8-[(3-bromophenyl)amide]-3-(3,4-dimethoxybenzylamide) | 573.6 |
| 229 | 3-[4-(3-chloropyridin-2-yl)piperazine-1-carbonyl]-1-oxa-2,8-diazaspiro[4.5]dec-2-ene-8-carboxylic acid-(4- | |
| | pentafluorosulphanylphenyl)amide | |
| 230 | N3-((5-methylfuran-2-yl)methyl)-N8-(4-(trifluoromethyl)phenyl)-1-oxa-2,8-diazaspiro[4.5]dec-2-ene-3,8-dicarboxamide | 465.1 |
| 231 | N8-(4-methoxyphenyl)-N3-((5-methylfuran-2-yl)methyl)-1-oxa-2,8-diazaspiro[4.5]dec-2-ene-3,8-dicarboxamide | 427.7 |
| 232 | N8-(4-chlorophenyl)-N3-((5-methylfuran-2-yl)methyl)-1-oxa-2,8-diazaspiro[4.5]dec-2-ene-3,8-dicarboxamide | 431.2 |
| 233 | N3-(4-(3-chloropyridin-2-yl)piperazin-1-yl)-N8-(3,4-dichlorophenyl)-1-oxa-2,8-diazaspiro[4.5]dec-2-ene-3,8- | 566.1 |
| | dicarboxamide | |
| 234 | N3-(4-(3-chloropyridin-2-yl)piperazin-1-yl)-N8-(4-methoxyphenyl)-1-oxa-2,8-diazaspiro[4.5]dec-2-ene-3,8- | 528.4 |
| | dicarboxamide | |
| 235 | N8-(4-chlorobenzyl)-N3-((5-methylfuran-2-yl)methyl)-1-oxa-2,8-diazaspiro[4.5]dec-2-ene-3,8-dicarboxamide | 445.2 |
| 236 | N8-(3,4-dichlorobenzyl)-N3-((5-methylfuran-2-yl)methyl)-1-oxa-2,8-diazaspiro[4.5]dec-2-ene-3,8-dicarboxamide | 479.2 |
| 237 | N3-(4-chlorobenzyl)-N8-(3,4-dichlorobenzyl)-1-oxa-2,8-diazaspiro[4.5]dec-2-ene-3,8-dicarboxamide | 509.3 |
| 238 | N8-(3,4-dichlorobenzyl)-N3-(4-hydroxy-3-methoxybenzyl)-1-oxa-2,8-diazaspiro[4.5]dec-2-ene-3,8-dicarboxamide | 521.3 |
| 239 | N3-(4-tert-butylbenzyl)-N8-(3,4-dichlorobenzyl)-1-oxa-2,8-diazaspiro[4.5]dec-2-ene-3,8-dicarboxamide | 531.2 |
| 240 | N8-(3,4-dichlorobenzyl)-N3-(4-(trifluoromethyl)benzyl)-1-oxa-2,8-diazaspiro[4.5]dec-2-ene-3,8-dicarboxamide | 543.1 |
| 241 | 3-(4-(3-chloropyridin-2-yl)piperazine-1-carbonyl)-N-(3,4-dichlorobenzyl)-1-oxa-2,8-diazaspiro[4.5]dec-2-ene-8- | 565.4 |
| | carboxamide | |
| 242 | N3-(4-(3-chloropyridin-2-yl)piperazin-1-yl)-N8-(3,4-dichlorobenzyl)-1-oxa-2,8-diazaspiro[4.5]dec-2-ene-3,8- | 580.9 |
| | dicarboxamide | |
| 243 | N8-(3,4-dichlorophenyl)-N3-((5-methylfuran-2-yl)methyl)-1-oxa-2,8-diazaspiro[4.5]dec-2-ene-3,8-dicarboxamide | 465.2 |
| 244 | N8-(4-chlorophenyl)-N3-(4-hydroxy-3-methoxybenzyl)-1-oxa-2,8-diazaspiro[4.5]dec-2-ene-3,8-dicarboxamide | 473.5 |
| 245 | N8-(3,4-dichlorophenyl)-N3-(4-hydroxy-3-methoxybenzyl)-1-oxa-2,8-diazaspiro[4.5]dec-2-ene-3,8-dicarboxamide | 507.6 |
| 246 | N3-(4-chlorobenzyl)-N8-(3,4-dichlorophenyl)-1-oxa-2,8-diazaspiro[4.5]dec-2-ene-3,8-dicarboxamide | 495.6 |
| 247 | N3-(4-chlorobenzyl)-N8-(4-chlorophenyl)-1-oxa-2,8-diazaspiro[4.5]dec-2-ene-3,8-dicarboxamide | 461.2 |
| 248 | N3-(4-hydroxy-3-methoxybenzyl)-N8-(4-(trifluoromethyl)phenyl)-1-oxa-2,8-diazaspiro[4.5]dec-2-ene-3,8-dicarboxamide | 507.3 |
| 249 | N3-(4-hydroxy-3-methoxybenzyl)-N8-(4-methoxyphenyl)-1-oxa-2,8-diazaspiro[4.5]dec-2-ene-3,8-dicarboxamide | 469.8 |
| 250 | N3-(4-chlorobenzyl)-N8-(4-(trifluoromethyl)phenyl)-1-oxa-2,8-diazaspiro[4.5]dec-2-ene-3,8-dicarboxamide | 495.3 |
| 251 | N8-(3,4-dichlorobenzyl)-N3-(3,4-dimethoxybenzyl)-1-oxa-2,8-diazaspiro[4.5]dec-2-ene-3,8-dicarboxamide | 535.6 |
| 252 | N3-(3,4-dimethoxybenzyl)-N8-(4-(trifluoromethyl)phenyl)-1-oxa-2,8-diazaspiro[4.5]dec-2-ene-3,8-dicarboxamide | 521.3 |
| 253 | 3-[4-(3-trifluoromethylpyridin-2-yl)piperazine-1-carbonyl]-1-oxa-2,8-diazaspiro[4.5]dec-2-ene-8-carboxylic acid-(4- | 643.3 |
| | pentafluorosulphanylphenyl)amide | |
| 254 | N-(4-tert-butylphenyl)-3-(4-(3-chloropyridin-2-yl)-2-methylpiperazine-1-carbonyl)-1-oxa-2,8-diazaspiro[4.5]dec-2-ene-8- | 553.2 |
| | carboxamide | |
| 255 | N-(4-tert-butylbenzyl)-3-(4-(3-chloropyridin-2-yl)piperazine-1-carbonyl)-1-oxa-2,8-diazaspiro[4.5]dec-2-ene-8- | 553.4 |
| | carboxamide | |

### Step 3: General directions for reacting amines of general formula IVa with aldehydes of general formula R¹—C(═O)—H

The respective aldehyde of general formula R¹—C(═O)—H (120 μmol in 0.5 mL MeOH) and then borane-pyridine complex (BH₃.C₅H₅N, 100 μmol in 0.5 mL MeOH) were added to a solution of the compound of general formula IVa (120 μmol in 0.5 mL MeOH) while stirring at RT. The reaction mixture was stirred for at least 16 hours at 64° C. and then mixed with 5% (percent by weight) hydrochloric acid solution in water (0.5 mL) while stirring. 10% (percent by weight) sodium hydroxide solution in water (1 mL) was added to the reaction mixture and the mixture was extracted three times with DCM (2 mL in each case). The combined organic phases were dried over MgSO₄ cartridges, the solvent was removed under vacuum and the residue purified by preparative HPLC in order to obtain the desired product of general formula I.

### Pharmacological Data

The affinity of the Spiro compounds according to the invention for the vanilloid receptor 1 (VR1/TRPV1 receptor) was determined as described hereinbefore.

| | | VR1 | | |
|---|---|---|---|---|
| | VR1 (human) | (human) | VR1 (rat) (% | VR1 (rat) |
| Compound | (% stimulation | (% inhibition | stimulation | (% inhibition |
| according to | compared to | compared to | compared to | compared to |
| the example | 10 μM CP) | 10 μM CP) | 10 μM CP) | 10 μM CP) |
| 1 | 4.85 | 33.56 | 6.37 | 34.23 |
| 9 | −0.48 | 26.23 | 0.23 | 45.63 |
| 11 | −0.15 | 37.28 | 11.45 | 41.27 |
| 16 | 0.40 | 0.77 | 0.00 | 8.65 |
| 17 | 19.54 | 53.69 | 19.83 | 46.89 |
| 18 | 0.67 | 11.17 | 4.68 | 34.24 |
| 22 | −0.66 | 8.37 | 2.79 | 43.32 |
| 28 | 0.08 | 27.56 | 0.63 | 45.91 |
| 34 | −0.13 | 51.91 | 6.21 | 86.21 |
| 51 | 13.15 | 63.38 | 18.63 | 98.80 |
| 52 | 47.48 | 45.14 | 38.53 | 55.84 |
| 56 | 5.26 | 41.94 | 30.25 | 63.33 |
| 58 | 72.92 | 84.90 | 19.54 | 72.54 |
| 61 | 0.08 | −15.08 | 0.47 | 47.05 |
| 63 | 2.59 | 15.81 | 24.47 | 78.90 |
| 68 | 16.35 | 24.87 | 6.55 | 58.09 |
| 70 | −0.18 | −0.43 | 0.35 | 67.41 |
| 71 | 34.68 | 42.03 | 31.24 | 57.20 |
| 116 | 42 | 77 | 59 | 95 |
| 228 | 7.07 | 48.14 | 17.25 | 100.28 |
| 229 | 10 | 37 | 45 | 94 |
| 259 | 2 | 14 | 8 | 94 |
| 261 | 19 | 66 | 70 | 94 |

1-40. (canceled)

41. A substituted spiro compound corresponding to formula I 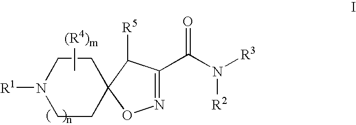
wherein
m is 0, 1, 2, 3 or 4,
n is 0, 1 or 2,
R¹ represents
a linear or branched, saturated or unsaturated, unsubstituted or mono- or polysubstituted aliphatic group optionally having at least one heteroatom as chain member;
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system;
an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, or
a —C(═S)—NR⁸R⁹ group, or
if m is not 0 and/or n is not 1 and/or R⁵ is not H,
can additionally represent a —C(═O)—NR⁶R⁷ group,
R² represents a linear or branched, saturated or unsaturated, unsubstituted or mono- or polysubstituted aliphatic group optionally having at least one heteroatom as chain member;
wherein the substituents of the aliphatic group can be independently selected from the group consisting of F, Cl, Br, I, —CN, —NO₂, —OH, —SH and —NH₂;
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, or
an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system;
R³ represents hydrogen,
a linear or branched, saturated or unsaturated, unsubstituted or mono- or polysubstituted aliphatic group optionally having at least one heteroatom as chain member,
wherein the substituents of the aliphatic group can be independently selected from the group consisting of F, Cl, Br, I, —CN, —NO₂, —OH, —SH and —NH₂;
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, or
an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system; or
R² and R³ form together with the nitrogen atom connecting them an unsubstituted or mono- or polysubstituted, unsaturated or saturated cycloaliphatic group which optionally has at least one further heteroatom as ring member and can be condensed with an unsubstituted or mono- or polysubstituted mono- or polycyclic ring system;
R⁴ represents a halogen group, a nitro group, a cyano group, an amino group, a hydroxy group, a thiol group, a carboxy group, a formyl group, an —NH—C(═O)—H group, an oxo group (═O), an —NH—R¹⁰ group, an —NR¹¹R¹² group, a —C(═O)—R¹³ group, a —C(═O)—O—R¹⁴ group, an —O—C(═O)—R¹⁵ group, an —NH—C(═O)—R¹⁶ group, an —NR¹⁷—C(═O)—R¹⁸ group, a —C(═O)—NH₂ group, a —C(═O)—NH—R¹⁹ group, a —C(═O)—NR²⁰R²¹ group, an —O—R²² group, an —S—R²³ group, or a linear or branched, saturated or unsaturated, unsubstituted or mono- or polysubstituted aliphatic group;
R⁵ represents hydrogen, a halogen group, a nitro group, a cyano group, an amino group, a hydroxy group, a thiol group, a carboxy group, a formyl group, an —NH—C(═O)—H group, an oxo group (═O), an —NH—R¹⁰ group, an —NR¹¹R¹² group, a —C(═O)—R¹³ group, a —C(═O)—O—R¹⁴ group, an —O—C(═O)—R¹⁵ group, an —NH—C(═O)—R¹⁶ group, an —NR¹⁷—C(═O)—R¹⁸ group, a —C(═O)—NH₂ group, a —C(═O)—NH—R¹⁹ group, a —C(═O)—NR²⁰R²¹ group, an —O—R²² group, an —S—R²³ group, a linear or branched, saturated or unsaturated, unsubstituted or mono- or polysubstituted aliphatic group, or
an unsubstituted or mono- or polysubstituted aryl or heteroaryl group which can be bound via a linear or branched, unsubstituted or mono- or polysubstituted alkylene, alkenylene or alkinylene group;
R⁶ and R⁸ each independently represent
a linear or branched, saturated or unsaturated, unsubstituted or mono- or polysubstituted aliphatic group optionally having at least one heteroatom as chain member,
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system and/or bridged with at least one linear or branched, unsubstituted or mono- or polysubstituted alkylene group, an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, a —C(═O)—R²⁴ group which can be bound via a linear or branched, unsubstituted or mono- or polysubstituted alkylene group, or a —C(═O)—O—R²⁵ group which can be bound via a linear or branched, unsubstituted or mono- or polysubstituted alkylene group;
R⁷ and R⁹ each independently represent
hydrogen,
a linear or branched, saturated or unsaturated, unsubstituted or mono- or polysubstituted aliphatic group optionally having at least one heteroatom as chain member,
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system and/or bridged with at least one linear or branched, unsubstituted or mono- or polysubstituted alkylene group, an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, a —C(═O)—R²⁴ group which can be bound via a linear or branched, unsubstituted or mono- or polysubstituted alkylene group, or a —C(═O)—O—R²⁵ group which can be bound via a linear or branched, unsubstituted or mono- or polysubstituted alkylene group; and
R¹⁰ to R²⁵ each independently represent
a linear or branched, saturated or unsaturated, unsubstituted or mono- or polysubstituted aliphatic group optionally having at least one heteroatom as chain member;
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, or
an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member;
or a salt or solvate thereof.

42. A compound according to claim 41, wherein
m is 0, 1, 2, 3 or 4,
n is 0, 1 or 2,
R¹ represents
a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system,
a —C(═S)—NR⁸R⁹ group, or
—(CHR²⁶)—X_{q}—(CHR²⁷)ᵣ—Yₛ—(CHR²⁸)ₜ-Zᵤ-R²⁹ wherein q=0 or 1, r=0 or 1, s=0 or 1, t=0 or 1, u=0 or 1, wherein X, Y and Z, independently of one another, can each represent O, S, NH, N(CH₃), N(C₂H₅) or N[CH(CH₃)₂], or if m is not 0 and/or n unequal to 1 and/or R⁵ unequal to H, additionally a —C(═O)—NR⁶R⁷ group,
R² represents
a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system, or
—(CHR³⁰)—Xᵥ—(CHR³¹)_{w}—Yₓ—(CHR³²)_{y}-Z_{z}-R³³ wherein v=0 or 1, w=0 or 1, x=0 or 1, y=0 or 1, z=0 or 1, wherein X, Y and Z each independently represent O, S, NH, N(CH₃), N(C₂H₅) or N[CH(CH₃)₂];
R³ represents
hydrogen;
a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system, or
—(CHR³⁰)—Xᵥ—(CHR³¹)_{w}—Yₓ—(CHR³²)_{y}-Z_{z}-R³³ wherein v=0 or 1, w=0 or 1, x=0 or 1, y=0 or 1, z=0 or 1, wherein X, Y and Z each independently represent O, S, NH, N(CH₃), N(C₂H₅) or N[CH(CH₃)₂]; or
R² and R³ together with the nitrogen atom to which they are bound form a saturated or unsaturated, optionally substituted 4, 5, 6, 7, 8 or 9-membered heterocycloaliphatic ring which optionally may be condensed with a saturated, unsaturated or aromatic, optionally substituted mono- or polycyclic ring system,
R⁴ represents a halogen group, a nitro group, a cyano group, an amino group, a hydroxy group, a thiol group, a carboxy group, a formyl group, an —NH—C(═O)—H group, an oxo group (═O), an —NH—R¹⁰ group, an —NR¹¹R¹² group, a —C(═O)—R¹³ group, a —C(═O)—O—R¹⁴ group, an —O—C(═O)—R¹⁵ group, an —NH—C(═O)—R¹⁶ group, an —NR¹⁷—C(═O)—R¹⁸ group, a —C(═O)—NH₂ group, a —C(═O)—NH—R¹⁹ group, a —C(═O)—NR²⁰R²¹ group, an —O—R²² group, an —S—R²³ group, or a linear or branched, saturated or unsaturated, optionally substituted C₁₋₁₀ aliphatic group;
R⁵ represents hydrogen, a halogen group, a nitro group, a cyano group, an amino group, a hydroxy group, a thiol group, a carboxy group, a formyl group, an —NH—C(═O)—H group, an oxo group (═O), an —NH—R¹⁰ group, an —NR¹¹R¹² group, a —C(═O)—R¹³ group, a —C(═O)—O—R¹⁴ group, an —O—C(═O)—R¹⁵ group, an —NH—C(═O)—R¹⁶ group, an —NR¹⁷—C(═O)—R¹⁸ group, a —C(═O)—NH₂ group, a —C(═O)—NH—R¹⁹ group, a —C(═O)—NR²⁰R²¹ group, an —O—R²² group, an —S—R²³ group, a linear or branched, saturated or unsaturated, optionally substituted C₁₋₁₀ aliphatic group;
an optionally substituted 5 to 14-membered aryl or heteroaryl group, or —(CH₂)ₐₐ—R³⁴ wherein aa=1, 2, 3 or 4;
R⁶ and R⁸ each independently represent
a linear or branched, saturated or unsaturated, optionally substituted C₁₋₂₀ aliphatic group;
an unsaturated or saturated, optionally substituted 3, 4, 5, 6, 7, 8, 9, 10, 11 or 12-membered cycloaliphatic group which optionally may be condensed with a saturated, unsaturated or aromatic, optionally substituted mono- or polycyclic ring system and/or bridged with one or two linear or branched, optionally substituted C₁₋₅ alkylene groups;
an optionally substituted 5 to 14-membered aryl or heteroaryl group which optionally may be condensed with a saturated or unsaturated, optionally substituted mono- or polycyclic ring system;
—(CHR³⁵)—(CH₂)_{bb}—(CH₂)_{cc}—C(═O)—R²⁴ wherein bb=0 or 1 and cc=0 or 1;
—(CHR³⁶)—(CH₂)_{dd}—(CH₂)ₑₑ—C(═O)—O—R²⁵ wherein dd=0 or 1 and ee=0 or 1; or
(CR³⁷R³⁸)—X_{ff}—(CHR³⁹)_{gg}—Yₕₕ—(CHR⁴⁰)ⱼⱼ-Zₖₖ-R⁴¹
wherein ff=0 or 1, gg=0 or 1, hh=0 or 1, ii=0 or 1, kk=0 or 1, wherein X, Y and Z each independently represent O, S, NH, N(CH₃), N(C₂H₅) or N[CH(CH₃)₂];
R⁷ and R⁹ each independently represent
hydrogen;
a linear or branched, saturated or unsaturated, optionally substituted C₁₋₂₀ aliphatic group;
an unsaturated or saturated, optionally substituted 3, 4, 5, 6, 7, 8, 9, 10, 11 or 12-membered cycloaliphatic group which optionally may be condensed with a saturated, unsaturated or aromatic, optionally substituted mono- or polycyclic ring system and/or bridged with one or two linear or branched, optionally substituted C₁₋₅ alkylene groups;
an optionally substituted 5 to 14-membered aryl or heteroaryl group which optionally may be condensed with a saturated or unsaturated, optionally substituted mono- or polycyclic ring system;
—(CHR³⁵)—(CH₂)_{bb}—(CH₂)_{cc}—C(═O)—R²⁴ wherein bb=0 or 1 and cc=0 or 1;
—(CHR³⁶)—(CH₂)_{dd}—(CH₂)ₑₑ—C(═O)—O—R²⁵ wherein dd=0 or 1 and ee=0 or 1; or
—(CR³⁷R³⁸)—X_{ff}—(CHR³⁹)_{gg}—Yₕₕ—(CHR⁴⁰)ⱼⱼ-Zₖₖ-R⁴¹
wherein ff=0 or 1, gg=0 or 1, hh=0 or 1, jj=0 or 1, kk=0 or 1, wherein X, Y and Z each independently represent O, S, NH, N(CH₃), N(C₂H₅) or N[CH(CH₃)₂];
R¹⁰ to R²⁵ each independently represent a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system, or
—(CH₂)ₘₘ—R⁴² wherein mm is 1, 2 or 3;
R²⁶, R²⁷, R²⁸, R³⁰, R³¹, R³², R³⁵, R³⁶, R³⁷, R³⁸, R³⁹ and R⁴⁰ each independently represent
hydrogen;
a linear or branched, saturated or unsaturated, optionally substituted C₁₋₁₀ aliphatic group, or
an optionally substituted 5 to 14-membered aryl or heteroaryl group which optionally may be condensed with a saturated or unsaturated, optionally substituted mono- or polycyclic ring system;
R²⁹, R³³ and R⁴¹ each independently represent a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which can be bridged with 1, 2, 3, 4 or 5 linear or branched, optionally substituted C₁₋₅ alkylene groups and/or condensed with a saturated, unsaturated or aromatic, optionally substituted mono- or polycyclic ring system; or
an optionally substituted 5 to 14-membered aryl or heteroaryl group which optionally may be condensed with a saturated or unsaturated, optionally substituted mono- or polycyclic ring system; and
R³⁴ and R⁴² each independently represent an optionally substituted 5 to 14-membered aryl or heteroaryl group;
wherein
the above-mentioned C₁₋₁₀ aliphatic groups and C₁₋₂₀ aliphatic groups can in each case optionally be substituted with 1, 2, 3, 4, 5, 6, 7, 8 or 9 substituents independently selected from the group consisting of F, Cl, Br, I, —CN, —NO₂, —OH, —SH and —NH₂;
the above-mentioned cycloaliphatic groups and heterocycloaliphatic groups can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents independently selected from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl;
the above-mentioned cycloaliphatic groups can in each case optionally have 1, 2, 3, 4 or 5 heteroatom(s) independently selected from the group consisting of oxygen, nitrogen and sulphur as ring member(s);
the above-mentioned heterocycloaliphatic groups can in each case optionally have 1, 2 or 3 additional heteroatom(s) independently selected from the group consisting of oxygen, nitrogen and sulphur as ring member(s);
the rings of the above-mentioned mono- or polycyclic ring systems can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl;
the rings of the above-mentioned mono- or polycyclic ring systems each have 5, 6 or 7 members and can in each case optionally have 1, 2, 3, 4 or 5 heteroatom(s) as ring member(s) which are independently selected from the group consisting of oxygen, nitrogen and sulphur;
the above-mentioned aryl or heteroaryl groups can in each case optionally be substituted with 1, 2, 3, 4 or 5 substituents independently selected from the group consisting of F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents independently selected from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl;
the above-mentioned heteroaryl groups can in each case optionally have 1, 2, 3, 4 or 5 heteroatom(s) independently selected from the group consisting of oxygen, nitrogen and sulphur as ring member(s); and
the above-mentioned C₁₋₅ alkylene groups can in each case optionally be substituted with 1, 2, 3, 4 or 5 substituents independently selected from the group consisting of F, Cl, Br, —OH, —SH, —NH₂, —CN and NO₂,

43. A compound according to claim 41, wherein R¹ represents
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a cyclic group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl and (1,2,3,4)-tetrahydroquinazolinyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
an aryl group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N— (CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a —C(═S)—NR⁸R⁹ group; or
—(CHR²⁶)—R²⁹; —(CHR²⁶)—(CHR²⁷)—R²⁹; —(CHR²⁶)— (CHR²⁷)—O—R²⁹; —(CHR²⁶)—(CHR²⁷)—(CHR²⁸)—R²⁹; —(CHR²⁶)—(CHR²⁷)—S—(CHR²⁸)—R²⁹; —(CHR²⁶)—(CHR²⁷)—(CHR²⁸)—N(CH₃)—R²⁹, or
if m is not 0 and/or n is not 1 and/or R⁵ is not H, R¹ may also represent a —C(═O)—NR⁶R⁷ group.

44. A compound according to claim 41, wherein R² represents
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl and (1,2,3,4)-tetrahydroquinazolinyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl; or
—(CHR³⁰)—R³³; —(CHR³⁰)—(CHR³¹)—R³³; —(CHR³⁰)—(CHR³¹)—O—R³³; —(CHR³⁰)—(CHR³¹)—(CHR³²)—R³³; —(CHR³⁰)—(CHR³¹)—S—(CHR³²)—R³³; or —(CHR³⁰)—(CHR³¹)—(CHR³²)—N(CH₃)—R³³.

45. A compound according to claim 41, wherein R³ represents hydrogen.

46. A compound according to claim 41, wherein R² and R³ together with the nitrogen atom to which they are bound form a ring selected from the group consisting of pyrrolidinyl, piperidinyl, (1,3,4,5)-tetrahydropyrido[4,3-b]indolyl, (3,4)-dihydro-1H-isoquinolinyl, (1,3,4,9)-tetrahydro-[b]-carbolinyl, imidazolidinyl, (1,3)-thiazolidinyl, piperazinyl, morpholinyl, azepanyl, diazepanyl and thiomorpholinyl, wherein the ring can in each case be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₁₁, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl.

47. A compound according to claim 41, wherein R⁴ represents a group selected from the group consisting of methyl, ethyl, n-propyl, isopropyl, n-butyl, sec-butyl, isobutyl, tert-butyl, n-pentyl, sec-pentyl, n-hexyl and n-heptyl, wherein the group can in each case optionally be substituted with 1, 2, 3, 4 or 5 substituents independently selected from the group consisting of F, Cl, Br, I, —CN, —NO₂, —OH, —SH and —NH₂.

48. A compound according to claim 41, wherein R⁵ represents
hydrogen;
a group selected from the group consisting of methyl, ethyl, n-propyl, isopropyl, n-butyl, sec-butyl, isobutyl, tert-butyl, n-pentyl, sec-pentyl, n-hexyl and n-heptyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of phenyl, naphthyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂ and —C(═O)—N—(C₂H₅)₂; or
—(CH₂)—R³⁴, —(CH₂)—(CH₂)—R³⁴ or —(CH₂)—(CH₂)—(CH₂)—R³⁴.

49. A compound according to claim 41, wherein R⁶ and R⁸ each independently represent
a group selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl, 2-methyl-1-propenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)—benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
—(CHR³⁵)—C(═O)—R²⁴ or —(CHR³⁵)—(CH₂)—C(═O)—R²⁴;
—(CHR³⁶)—C(═O)—O—R²⁵ or —(CHR³⁶)—(CH₂)—C(═O)—O—R²⁵; or
—(CR³⁷R³⁸)—R⁴¹, —(CR³⁷R³⁸)—(CHR³⁹)—R⁴¹, —(CR³⁷R³⁸)— (CHR³⁹)—O—R⁴¹, —(CR³⁷R³⁸)—(CHR³⁹)—(CHR⁴⁰)—R⁴¹, —(CR³⁷R³⁸)— (CHR³⁹)—(CHR⁴⁰)—O—R⁴¹ (CR³⁷R³⁸)—(CHR³⁹)—(CHR⁴⁰)—N(CH₃)—R⁴¹ or —(CR³⁷R³⁸)—(CHR³⁹)—(CHR⁴⁰)—N(C₂H₅)—R⁴¹.

50. A compound according to claim 41, wherein R⁷ and R⁹ each represent hydrogen.

51. A compound according to claim 41, wherein R¹⁰ to R²⁵ each independently represent
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl, wherein the group can in each case be substituted with 1 or 2 substituents independently selected from the group consisting of —C(═O)—O—CH₃, —C(═O)—O—C₂H₅, —C(═O)—O—C(CH₃)₃, —C(═O)—CH₃, —C(═O)—C₂H₅, —C(═O)—C(CH₃)₃, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, pyridinyl, phenyl and benzyl, wherein in each case the cyclic portion of the groups pyridinyl, —S(═O)₂ phenyl, phenyl and benzyl can be substituted with 1, 2, 3, 4 or 5 substituents independently selected from the group consisting of F, Cl, Br, methyl, ethyl, n-propyl, isopropyl, n-butyl, sec-butyl, isobutyl, tert-butyl, —O—CH₃ and —O—C₂H₅;
a group selected from the group consisting of phenyl, naphthyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl and pyrimidinyl, wherein the group can in each case optionally be substituted with 1, 2, 3, 4 or 5 substituents independently selected from the group consisting of F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl; or
—(CH₂)—R⁴² or —(CH₂)—(CH₂)—R⁴².

52. A compound according to claim 41, wherein R²⁶, R²⁷, R²⁸, R³⁰, R³¹, R³², R³⁵, R³⁶, R³⁷, R³⁸, R³⁹ and R⁴⁰ each independently represent
hydrogen;
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl; or
a phenyl group which optionally may be substituted with 1, 2, 3, 4 or 5 substituents independently selected from the group consisting of F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl.

53. A compound according to claim 41, wherein R²⁹, R³³ and R⁴¹ each independently represent
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl and (1,2,3,4)-tetrahydroquinazolinyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl; or
a group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl; wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl.

54. A compound according to claim 41, wherein R³⁴ and R⁴² each independently represent a group selected from the group consisting of phenyl, naphthyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl and pyrimidinyl;
wherein the group optionally may be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, and —C(═O)—C(CH₃)₃.

55. A compound according to claim 41, wherein m is 0.

56. A compound according to claim 41, wherein n is 1.

57. A compound according to claim 41, wherein
m is 0, 1, 2, 3 or 4;
n is 1;
R¹ represents
a group selected from the group consisting of methyl, ethyl, n-propyl, isopropyl, n-butyl, sec-butyl, isobutyl, tert-butyl, n-pentyl, sec-pentyl, n-hexyl, n-heptyl and n-octyl;
a (hetero)cycloaliphatic group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl and thiomorpholinyl;
a group selected from the group consisting of indolyl, isoindolyl, benzo[b]furanyl, benzo[b]thiophenyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —NH—S(═O)₂—CH₃, —NH—S(═O)₂—C₂H₅, —S(═O)₂—NH—CH₃, —S(═O)₂—NH—C₂H₅;
a —C(═S)—NR⁸R⁹ group; —(CHR²⁶)—R²⁹; —(CHR²⁶)—(CHR²⁷)—R²⁹; —(CHR²⁶)— (CHR²⁷)—(CHR²⁸)—R²⁹; or
if m is not 0 and/or n is not 1 and/or R⁵ is not H, R¹ can also represent a —C(═O)—NR⁶R⁷ group,
R² represents
a group selected from the group consisting of methyl, ethyl, n-propyl, isopropyl, n-butyl, sec-butyl, isobutyl, tert-butyl, n-pentyl, sec-pentyl n-hexyl and n-heptyl;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl and thiomorpholinyl, wherein the group can in each case be substituted with 1 or 2 substituents independently selected from the group consisting of pyridinyl, pyridazinyl, phenyl and benzyl, wherein in each case the cyclic portion of the groups pyridinyl, pyridazinyl, phenyl and benzyl can be substituted with 1, 2, 3, 4 or 5 substituents independently selected from the group consisting of —CF₃, F, Cl, Br, methyl, ethyl, n-propyl, isopropyl, n-butyl, sec-butyl, isobutyl, tert-butyl, —O—CH₃ and —O—C₂H₅;
a group selected from the group consisting of phenyl, naphthyl, thiophenyl, furanyl, pyrrolyl, pyrazolyl, pyrazinyl and pyridinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —S(═O)₂—CH₃, —S(═O)₂—C₂H₅, —NH—S(═O)₂—CH₃, —NH—S(═O)₂—C₂H₅, —S(═O)₂—NH—CH₃ and —S(═O)₂—NH—C₂H₅; or
—(CHR³⁰)—R³³; —(CHR³⁰)—(CHR³¹)—R³³ or —(CHR³⁰)—(CHR³¹)—(CHR³²)—R³³;
R³ represents hydrogen; or
R² and R³ together with the nitrogen atom to which they are bound form a ring selected from the group consisting of pyrrolidinyl, piperidinyl, piperazinyl, 3-methylpiperazinyl, 2-methylpiperazinyl, (3,5)-dimethylpiperazinyl, (2,6)-dimethylpiperazinyl, morpholinyl and thiomorpholinyl, wherein the ring can in each case be substituted with 1 or 2 substituents independently selected from the group consisting of —C(═O)—O—CH₃, —C(═O)—O—C₂H₅, —C(═O)—O—C(CH₃)₃, —C(═O)—CH₃, —C(═O)—C₂H₅, —C(═O)—C(CH₃)₃, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, pyridinyl, pyridazinyl, phenyl and benzyl, wherein in each case the cyclic portion of the groups pyridinyl, —S(═O)₂ phenyl, pyridazinyl, phenyl and benzyl can be substituted with 1, 2, 3, 4 or 5 substituents independently selected from the group consisting of —CF₃, F, Cl, Br, methyl, ethyl, n-propyl, isopropyl, n-butyl, sec-butyl, isobutyl, tert-butyl, —O—CH₃ and —O—C₂H₅;
R⁴ represents a group selected from the group consisting of methyl, ethyl, n-propyl, isopropyl, n-butyl, sec-butyl, isobutyl, tert-butyl, n-pentyl, sec-pentyl, n-hexyl and n-heptyl;
R⁵ represents
hydrogen;
a group selected from the group consisting of methyl, ethyl, n-propyl, isopropyl, n-butyl, sec-butyl, isobutyl, tert-butyl, n-pentyl, sec-pentyl, n-hexyl and n-heptyl;
a phenyl group which can in each case be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl and tert-butyl; or
—(CH₂)—R³⁴;
R⁶ and R⁸ each independently represent
a group selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl, 2-methyl-1-propenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl and Br;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl;
a group selected from the group consisting of phenyl, naphthyl, thiophenyl, furanyl, pyrrolyl, pyrazolyl, pyranyl, triazolyl, pyridinyl, imidazolyl, thiazolyl, oxazolyl, isoxazolyl, pyridazinyl, pyrazinyl and pyrimidinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
—(CHR³⁶)—C(═O)—O—R²⁵ or —(CHR³⁶)—(CH₂)—C(═O)—O—R²⁵; or
(CR³⁷R³⁸)—R⁴¹, —(CR³⁷R³⁸)—(CHR³⁹)—R⁴¹, —(CR³⁷R³⁸) (CHR³⁹)—O—R⁴¹, —
(CR³⁷R³⁸)—(CHR³⁹)—(CHR⁴⁰)—R⁴¹, —(CR³⁷R³⁸)— (CHR³⁹)—(CHR⁴⁰)—O—R⁴¹, —(CR³⁷R³⁸)—(CHR³⁹)—(CHR⁴⁰)—N(CH₃)—R⁴¹ or —(CR³⁷R³⁸)—(CHR³⁹)—(CHR⁴⁰)—N(C₂H₅)—R⁴¹;
R⁷ and R⁹ each represent hydrogen;
R²⁵ represents a group selected from the group consisting of methyl, ethyl, n-propyl, isopropyl, n-butyl, sec-butyl, isobutyl and tert-butyl;
R²⁶, R²⁷, R²⁸, R³⁰, R³¹, R³², R³⁶, R³⁷, R³⁸, R³⁹ and R⁴⁰ each independently represent hydrogen;
a group selected from the group consisting of methyl, ethyl, n-propyl, isopropyl, n-butyl, sec-butyl, isobutyl and tert-butyl; or
a phenyl group which can be substituted with 1, 2, 3, 4 or 5 substituents independently selected from the group consisting of F, Cl, Br, I, —CN, —CF₃, —O—CH₃, —O—C₂H₅, —NO₂, —O—CF₃, methyl, ethyl, n-propyl, isopropyl, n-butyl, sec-butyl, isobutyl and tert-butyl;
R²⁹ and R³³ each independently represent a group selected from the group consisting of phenyl, naphthyl, thiophenyl, furanyl, pyrrolyl, pyrazolyl, pyrazinyl and pyridinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —S(═O)₂—CH₃, —S(═O)₂—C₂H₅, —NH—S(═O)₂—CH₃, —NH—S(═O)₂—C₂H₅, —S(═O)₂—NH—CH₃ and —S(═O)₂—NH—C₂H₅;
R³⁴ represents a phenyl group which can be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl and tert-butyl; and
R⁴¹ represents
a group selected from the group consisting of methyl, ethyl, n-propyl, isopropyl, n-butyl, sec-butyl, isobutyl and tert-butyl;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl and dithiolanyl; or
a group selected from the group consisting of phenyl, naphthyl, (1,3)-benzodioxolyl, (1,4)-benzodioxanyl, thiophenyl and furanyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅ and —C(═O)—C(CH₃)₃.

58. A compound according to claim 41, wherein
m is 0;
n is 1;
R¹ represents
a group selected from the group consisting of benzimidazolinyl, benzoxazolyl, benzisoxazolyl and benzothiazolyl, wherein the group can in each case be substituted with 1, 2, 3, 4 or 5 substituents independently selected from the group consisting of F, Cl, Br, —CF₃, —O—CF₃, —S—CF₃, methyl, ethyl, n-propyl, isopropyl, n-butyl, sec-butyl, isobutyl and tert-butyl; or
a —C(═S)—NR⁸R⁹ group;
R² represents
a group selected from the group consisting of phenyl, naphthyl and pyridinyl, wherein the group can in each case be substituted with 1, 2 or 3 substituents independently selected from the group consisting of F, Cl, Br, —OH, —O—CH₃, —O—C₂H₅, —NH—S(═O)₂—CH₃ and —NH—S(═O)₂—C₂H₅;
a piperazinyl group which on a nitrogen atom can be substituted with a substituent selected from the group consisting of pyridinyl, pyridazinyl, phenyl and benzyl, wherein in each case the cyclic portion of the substituents pyridinyl, pyridazinyl, phenyl and benzyl can be substituted with 1, 2 or 3 substituents independently selected from the group consisting of —CF₃, F, Cl, Br, methyl, ethyl, n-propyl, isopropyl, n-butyl, sec-butyl, isobutyl and tert-butyl; or
—(CHR³⁰)—R³³; —(CHR³⁰)—(CHR³¹)—R³³ or —(CHR³⁰)—(CHR³¹)—(CHR³²)—R³³;
R³ represents hydrogen; or
R² and R³ together with the nitrogen atom to which they are bound form a ring selected from the group consisting of 3-methylpiperazinyl, 2-methylpiperazinyl, (3,5)-dimethylpiperazinyl, (2,6)-dimethylpiperazinyl and piperazinyl which on a nitrogen atom can be substituted with a substituent selected from the group consisting of pyridinyl, pyridazinyl, phenyl and benzyl, wherein in each case the cyclic portion of the substituents pyridinyl, pyridazinyl, phenyl and benzyl can be substituted with 1, 2 or 3 substituents independently selected from the group consisting of —CF₃, F, Cl, Br, methyl, ethyl, n-propyl, isopropyl, n-butyl, sec-butyl, isobutyl and tert-butyl;
R⁵ represents hydrogen,
R⁶ and R⁸ each independently represent
a group selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl and 2-methyl-1-propenyl, wherein the group can in each case optionally be substituted with 1, 2, 3, 4 or 5 substituents independently selected from the group consisting of F, Cl and Br;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl and adamantyl;
a group selected from the group consisting of phenyl, naphthyl and pyridinyl, wherein the group can in each case optionally be substituted
with 1, 2, 3, 4 or 5 substituents independently selected from the group consisting of —SF₅, F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl, wherein in each case the cyclic portion of the groups cyclopentyl, cyclohexyl, —O-phenyl, —O-benzyl and phenyl can be substituted with 1, 2, 3, 4 or 5 substituents independently selected from the group consisting of F, Cl, Br, methyl, ethyl, n-propyl, isopropyl, n-butyl, sec-butyl, isobutyl and tert-butyl;
—(CHR³⁶)—C(═O)—O—R²⁵ or —(CHR³⁶)—(CH₂)—C(═O)—O—R²⁵; or
—(CR³⁷R³⁸)—R⁴¹, —(CR³⁷R³⁸)—(CHR³⁹)—R⁴¹, —(CR³⁷R³⁸)— (CHR³⁹)—O—R⁴¹, —(CR³⁷R³⁸)—(CHR³⁹)—(CHR⁴⁰)—R⁴¹, —(CR³⁷R³⁸)—(CHR³⁹)—(CHR⁴⁰)—O—R⁴¹, —(CR³⁷R³⁸)—(CHR³⁹)—(CHR⁴⁰)—N(CH₃)—R⁴¹ or —(CR³⁷R³⁸)—(CHR³⁹)— (CHR⁴⁰)—N(C₂H₅)—R⁴¹;
R⁷ and R⁹ each represent hydrogen;
R²⁵ represents a group selected from the group consisting of methyl, ethyl, n-propyl, isopropyl, n-butyl, sec-butyl, isobutyl and tert-butyl;
R³⁰, R³¹, R³², R³⁹ and R⁴⁰ each represent hydrogen;
R³³ represents a group selected from the group consisting of phenyl, naphthyl, thiophenyl and furanyl, wherein the group can in each case optionally be substituted with 1, 2, 3, 4 or 5 substituents independently selected from the group consisting of —CF₃, F, Cl, Br, —OH, —O—CH₃, —O—C₂H₅, methyl, ethyl, n-propyl, isopropyl, n-butyl, sec-butyl, isobutyl, tert-butyl, —NH—S(═O)₂—CH₃ and —NH—S(═O)₂—C₂H₅;
R³⁶ represents hydrogen or a group selected from the group consisting of methyl, ethyl, n-propyl, isopropyl, n-butyl, sec-butyl, isobutyl and tert-butyl;
R³⁷ and R³⁸ each independently represent
hydrogen;
a group selected from the group consisting of methyl, ethyl, n-propyl, isopropyl, n-butyl, sec-butyl, isobutyl and tert-butyl; or
a phenyl group;
and
R⁴¹ represents
a group selected from the group consisting of methyl, ethyl, n-propyl, isopropyl, n-butyl, sec-butyl, isobutyl and tert-butyl;
a group selected from the group consisting of tetrahydrofuranyl, tetrahydrothiophenyl, pyrrolidinyl, morpholinyl, piperidinyl and piperazinyl; or
a group selected from the group consisting of phenyl, naphthyl, (1,3)-benzodioxolyl, (1,4)-benzodioxanyl, thiophenyl and furanyl, wherein the group can in each case optionally be substituted with 1, 2, 3, 4 or 5 substituents independently selected from the group consisting of F, Cl, Br, —OH, —O—CH₃, —O—C₂H₅, methyl, ethyl, n-propyl, isopropyl, n-butyl, sec-butyl, isobutyl and tert-butyl.

59. A compound according to claim 41, wherein in a FLIPR assay at a concentration of 10 μM said compound exhibits inhibition of the Ca²⁺ ion inflow in dorsal root ganglia of rats of at least 10%, preferably of at least 30%, particularly preferably of at least 50%, most particularly preferably of at least 70%, even more preferably of at least 90%, compared to the maximum achievable inhibition of the Ca²⁺ ion inflow with capsaicin in a concentration of 10 μM.

60. A process for preparing a substituted spiro compound of formula I according to claim 41, wherein a compound of formula II, 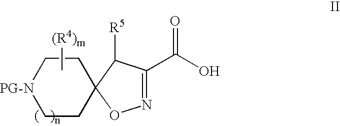
wherein R⁴, R⁵, m and n are as defined in claim 41 and PG represents a protective group, preferably a benzyloxycarbonyl or tert-butyloxycarbonyl group, is reacted in a reaction medium in the presence of at least one coupling reagent, optionally in the presence of at least one base, with a compound of formula HNR²R³, wherein R² and R³ are as defined in claim 41, to form at least one compound of formula III, 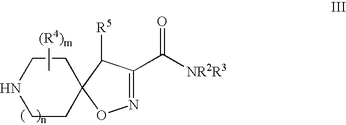
wherein R², R³, R⁴, R⁵, m, n and PG are as defined above, and the at least one compound of formula III is optionally purified and/or isolated and
at least one compound of formula III is reacted in a reaction medium in the presence of at least one acid, preferably in the presence of at least one inorganic or organic acid selected from the group consisting of hydrochloric acid, sulphuric acid, acetic acid and trifluoroacetic acid, or in the presence of hydrogen and a catalyst, preferably a catalyst based on palladium or platinum, to form at least one compound of formula IV, 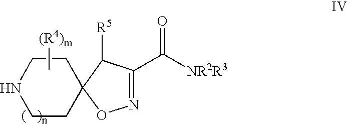
wherein R², R³, R⁴, R⁵, m and n are as defined above, and the at least one compound of formula IV is optionally purified and/or isolated and
at least one compound of formula IV is reacted in a reaction medium with at least one isocyanate of formula R⁶—N═C═O, wherein R⁶ is as defined in claim 41, optionally in the presence of at least one base, preferably in the presence of at least one base selected from the group consisting of triethylamine, 4,4-dimethylaminopyridine and diisopropylethylamine, to form at least one compound of formula I, wherein R², R³, R⁴, R⁵, m and n are as defined above and R¹ represents —C(═O)—NR⁶R⁷, wherein R⁶ is as defined above and R⁷ represents hydrogen, and the at least one compound of formula I is optionally purified and/or isolated or
at least one compound of formula IV is reacted in a reaction medium with at least one isothiocyanate of formula S═C═N—R⁸, wherein R⁸ is as defined in claim 41, optionally in the presence of at least one base, preferably in the presence of at least one base selected from the group consisting of triethylamine, 4,4-dimethylaminopyridine and diisopropylethylamine, to form at least one compound of formula I,
wherein R², R³, R⁴, R⁵, m and n are as defined above and R¹ represents —C(═S)—N—R⁸R⁹, wherein R⁸ is as defined above and R⁹ represents hydrogen, and the at least one compound of formula I is optionally purified and/or isolated
and optionally at least one compound of formula I, wherein R², R³, R⁴, R⁵, m and n are as defined above and R¹ represents —C(═O)—NR⁶R⁷ or —C(═S)—N—R⁸R⁹, wherein R⁷ and R⁹ each represent hydrogen, is reacted in a reaction medium, in the presence of at least one base, preferably in the presence of at least one metal hydride salt or a metal alcoholate salt, particularly preferably in the presence of a metal hydride salt or a metal alcoholate salt selected from the group consisting of sodium hydride, potassium hydride, potassium tert-butanolate, sodium tert-butanolate, potassium methanolate, sodium methanolate, sodium ethanolate and potassium ethanolate, with at least one compound of formula LG-R⁷ or of formula LG-R⁹, wherein LG represents a leaving group, preferably a halogen atom, particularly preferably a chlorine atom, and R⁷ and R⁹ are as defined above except for hydrogen, to form at least one compound of formula I, wherein R¹ to R⁵, m and n are as defined above, and the at least one compound of formula I is optionally purified and/or isolated, or
at least one compound of formula IV is reacted in a reaction medium, in the presence of at least one base, preferably in the presence of at least one metal hydride salt, particularly preferably in the presence of sodium and/or potassium hydride, with at least one compound of formula LG-R¹, wherein R¹ is as defined in claim 41, except for —C(═O)—NR⁶R⁷ and —C(═S)—NR⁸R⁹, and LG represents a leaving group, preferably a halogen atom, particularly preferably a chlorine atom, to form at least one compound of formula I, wherein R¹ to R⁵, m and n are as defined above, and the at least one compound of formula I is optionally purified and/or isolated or
at least one compound of formula IV is reacted in a reaction medium, in the presence of at least one reducing agent, with at least one compound of formula R¹—C(═O)—H, wherein R¹ is as defined in claim 41, except for —C(═O)—NR⁶R⁷ and —C(═S)—NR⁸R⁹, to form at least one compound of formula I, wherein R¹ to R⁵, m and n are as defined above, and the at least one compound of formula I is optionally purified and/or isolated.

61. A pharmaceutical composition comprising a compound according to claim 41, and at least one physiologically compatible excipient.

62. A method of treating or inhibiting a disorder or disease state at least partially mediated by vanilloid receptor 1 (VR1/TRPV1) in a subject in need thereof, said method comprising administering to said subject an effective amount of a compound according to claim 41.

63. A method according to claim 62, wherein said disorder or disease state is selected from the group consisting of pain, preferably of pain selected from the group consisting of acute pain, chronic pain, neuropathic pain and visceral pain; arthralgia; migraine; depression; neuropathy; nerve injuries; neurodegenerative diseases, preferably selected from the group consisting of multiple sclerosis, Alzheimer's disease, Parkinson's disease and Huntington's disease; cognitive dysfunctions, preferably cognitive deficiencies, particularly preferably paramnesia; epilepsy; respiratory diseases, preferably selected from the group consisting of asthma and pneumonia; coughing; urinary incontinence; OAB (overactive bladder); stomach ulcers; irritable bowel syndrome; strokes; irritations of the eyes; irritations of the skin; neurotic skin diseases; inflammatory diseases, preferably intestinal inflammations; diarrhoea; pruritus; eating disorders, preferably selected from the group consisting of bulimia, cachexia, anorexia and obesity; medication dependency; medication abuse; withdrawal symptoms in medication dependency; development of tolerance to medication, preferably to natural or synthetic opioids; drug addiction; drug abuse; withdrawal symptoms in drug addiction; alcohol addiction; alcohol abuse and withdrawal symptoms in alcohol addiction; for diuresis; for antinatriuresis; for influencing the cardiovascular system; for increasing vigilance; for increasing libido; for modulating motor activity; for anxiolysis; for local anaesthetics and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil (DA-5018), in a subject in need thereof, said method comprising administering to said subject a therapeutically effective amount of a compound according to claim 41.

64. A method of treating or inhibiting a disorder or disease state at least partially mediated by vanilloid receptor 1 (VR1/TRPV1) in a subject in need thereof, said method comprising administering to said subject an effective amount of a spiro compound corresponding to formula I 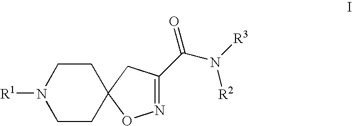
wherein
R¹ represents a —C(═O)—NR⁶R⁷ group,
R² represents
a linear or branched, saturated or unsaturated, unsubstituted or mono- or polysubstituted aliphatic group optionally having at least one heteroatom as chain member; wherein the substituents of the aliphatic group can be independently selected from the group consisting of F, Cl, Br, I, —CN, —NO₂, —OH, —SH and —NH₂;
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system; or
an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system;
R³ represents
hydrogen;
a linear or branched, saturated or unsaturated, unsubstituted or mono- or polysubstituted aliphatic group optionally having at least one heteroatom as chain member,
wherein the substituents of the aliphatic group can be independently selected from the group consisting of F, Cl, Br, I, —CN, —NO₂, —OH, —SH and —NH₂;
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system,
or an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system; or
R² and R³ form together with the nitrogen atom connecting them an
unsubstituted or mono- or polysubstituted, unsaturated or saturated cycloaliphatic group which optionally has at least one further heteroatom as ring member and can be condensed with an unsubstituted or mono- or polysubstituted mono- or polycyclic ring system;
R⁶ represents
a linear or branched, saturated or unsaturated, unsubstituted or mono- or polysubstituted aliphatic group optionally having at least one heteroatom as chain member,
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system and/or bridged with at least one linear or branched, unsubstituted or mono- or polysubstituted alkylene group, an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, a —C(═O)—R²⁴ group which can be bound via a linear or branched, unsubstituted or mono- or polysubstituted alkylene group, or a —C(═O)—O—R²⁵ group which can be bound via a linear or branched, unsubstituted or mono- or polysubstituted alkylene group;
R⁷ represents
hydrogen,
a linear or branched, saturated or unsaturated, unsubstituted or mono- or polysubstituted aliphatic group optionally having at least one heteroatom as chain member,
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system and/or bridged with at least one linear or branched, unsubstituted or mono- or polysubstituted alkylene group, an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, a —C(═O)—R²⁴ group which can be bound via a linear or branched, unsubstituted or mono- or polysubstituted alkylene group, or a —C(═O)—O—R²⁵ group which can be bound via a linear or branched, unsubstituted or mono- or polysubstituted alkylene group; and
R²⁴ and R²⁵ each independently represent
a linear or branched, saturated or unsaturated, unsubstituted or mono- or polysubstituted aliphatic group optionally having at least one heteroatom as chain member,
an unsubstituted or mono- or polysubstituted, unsaturated or saturated cycloaliphatic group which optionally has at least one heteroatom as ring member and can be bound via a linear or branched, unsubstituted or mono- or polysubstituted alkylene, alkenylene or alkinylene group optionally having at least one heteroatom as chain member and/or condensed with an unsubstituted or mono- or polysubstituted mono- or polycyclic ring system, or
an unsubstituted or mono- or polysubstituted aryl or heteroaryl group which can be bound via a linear or branched, unsubstituted or mono- or polysubstituted alkylene, alkenylene or alkinylene group optionally having at least one heteroatom as chain member;
or a physiologically compatible salt or solvate thereof.

65. A method according to claim 64, wherein said disorder or disease state is selected from the group consisting of acute pain; visceral pain; arthralgia; cognitive dysfunctions, preferably cognitive deficiencies, particularly preferably paramnesia; pneumonia; coughing; OAB (overactive bladder); irritable bowel syndrome (irritable bowel syndrom); irritations of the eyes; irritations of the skin; neurotic skin diseases; intestinal inflammations; development of tolerance to medication, preferably to natural or synthetic opioids; and/or for diuresis or for antinatriuresis and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

66. A method according to claim 64, wherein
R¹ represents a —C(═O)—NR⁶R⁷ group,
R² represents a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system, or
—(CHR³⁰)—Xᵥ—(CHR³¹)_{w}—Yₓ—(CHR³²)_{y}-Z_{z}-R³³ wherein v=0 or 1, w=0 or 1, x=0 or 1, y=0 or 1, z=0 or 1, wherein X, Y and Z each independently represent O, S, NH, N(CH₃), N(C₂H₅) or N[CH(CH₃)₂];
R³ represents
hydrogen;
a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system,
or —(CHR³⁰)—Xᵥ—(CHR³¹)_{w}—Yₓ—(CHR³²)_{y}-Z_{z}-R³³ wherein v=0 or 1, w=0 or 1, x=0 or 1, y=0 or 1, z=0 or 1, wherein X, Y and Z each independently represent O, S, NH, N(CH₃), N(C₂H₅) or N[CH(CH₃)₂]; or
R² and R³ form together with the nitrogen atom connecting them as ring member a saturated or unsaturated, optionally substituted 4, 5, 6, 7, 8 or 9-membered heterocycloaliphatic group which optionally may be condensed with a saturated, unsaturated or aromatic, optionally substituted mono- or polycyclic ring system,
R⁶ represents
a linear or branched, saturated or unsaturated, optionally substituted C₁₋₂₀ aliphatic group;
an unsaturated or saturated, optionally substituted 3, 4, 5, 6, 7, 8, 9, 10, 11 or 12-membered cycloaliphatic group which optionally may be condensed with a saturated, unsaturated or aromatic, optionally substituted mono- or polycyclic ring system and/or bridged with one or two linear or branched, optionally substituted C₁₋₅ alkylene groups;
an optionally substituted 5 to 14-membered aryl or heteroaryl group which optionally may be condensed with a saturated or unsaturated, optionally substituted mono- or polycyclic ring system;
—(CHR³⁵)—(CH₂)_{bb}—(CH₂)_{cc}—C(═O)—R²⁴ wherein bb=0 or 1 and cc=0 or 1;
(CHR³⁶)—(CH₂)_{dd}—(CH₂)ₑₑ—C(═O)—O—R²⁵ wherein dd=0 or 1 and ee=0 or 1; or
—(CR³⁷R³⁸)—X_{ff}—(CHR³⁹)_{gg}—Yₕₕ—(CHR⁴⁰)ⱼⱼ-Zₖₖ-R⁴¹ wherein ff=0 or 1, gg=0 or 1, hh=0 or 1, jj=0 or 1, kk=0 or 1,
wherein X, Y and Z each independently represent O, S, NH, N(CH₃), N(C₂H₅) or N[CH(CH₃)₂];
R⁷ represents
hydrogen;
a linear or branched, saturated or unsaturated, optionally substituted C₁₋₂₀ aliphatic group;
an unsaturated or saturated, optionally substituted 3, 4, 5, 6, 7, 8, 9, 10, 11 or 12-membered cycloaliphatic group which optionally may be condensed with a saturated, unsaturated or aromatic, optionally substituted mono- or polycyclic ring system and/or bridged with one or two linear or branched, optionally substituted C₁₋₅ alkylene groups;
an optionally substituted 5 to 14-membered aryl or heteroaryl group which optionally may be condensed with a saturated or unsaturated, optionally substituted mono- or polycyclic ring system;
—(CHR³⁵)—(CH₂)_{bb}—(CH₂)_{cc}—C(═O)—R²⁴ wherein bb=0 or 1 and cc=0 or 1;
—(CHR³⁶)—(CH₂)_{dd}—(CH₂)ₑₑ—C(═O)—O—R²⁵ wherein dd=0 or 1 and ee=0 or 1; or
—(CR³⁷R³⁸)—X_{ff}—(CHR³⁹)_{gg}—Yₕₕ—(CHR⁴⁰)ⱼⱼ-Zₖₖ-R⁴¹
wherein ff=0 or 1, gg=0 or 1, hh=0 or 1, jj=0 or 1, kk=0 or 1, wherein X, Y and Zeach independently represent O, S, NH, N(CH₃), N(C₂H₅) or N[CH(CH₃)₂];
R²⁴ and R²⁵ each independently represent
a linear or branched, saturated or unsaturated, optionally substituted C₁₋₁₀ aliphatic group;
an unsaturated or saturated, optionally substituted 3, 4, 5, 6, 7, 8 or 9-membered cycloaliphatic group which optionally may be condensed with a saturated, unsaturated or aromatic, optionally substituted mono- or polycyclic ring system;
an optionally substituted 5 to 14-membered aryl or heteroaryl group which optionally may be condensed with a saturated or unsaturated, optionally substituted mono- or polycyclic ring system,
or —(CH₂)ₘₘ—R⁴² wherein mm is 1, 2 or 3;
R³⁰, R³¹, R³², R³⁵, R³⁶, R³⁷, R³⁸, R³⁹ and R⁴⁰ each independently represent hydrogen;
a linear or branched, saturated or unsaturated, optionally substituted Cl aliphatic group, or
an optionally substituted 5 to 14-membered aryl or heteroaryl group which optionally may be condensed with a saturated or unsaturated, optionally substituted mono- or polycyclic ring system;
R³³ and R⁴¹ each independently represent a linear or branched, saturated or unsaturated, optionally substituted Cl aliphatic group;
an unsaturated or saturated, optionally substituted 3, 4, 5, 6, 7, 8 or 9-membered cycloaliphatic group which can be bridged with 1, 2, 3, 4 or 5 linear or branched, optionally substituted C₁₋₅ alkylene groups and/or condensed with a saturated, unsaturated or aromatic, optionally substituted mono- or polycyclic ring system; or
an optionally substituted 5 to 14-membered aryl or heteroaryl group which optionally may be condensed with a saturated or unsaturated, optionally substituted mono- or polycyclic ring system; and
R³⁴ and R⁴² each independently represent an optionally substituted 5 to 14-membered aryl or heteroaryl group;
wherein
the above-mentioned C₁₋₁₀ aliphatic groups and C₁₋₂₀ aliphatic groups can in each case optionally be substituted with 1, 2, 3, 4, 5, 6, 7, 8 or 9 substituents independently selected from the group consisting of F, Cl, Br, I, —CN, —NO₂, —OH, —SH and —NH₂;
the above-mentioned cycloaliphatic groups and heterocycloaliphatic groups can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents independently selected from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl,
the above-mentioned cycloaliphatic groups can in each case optionally have 1, 2, 3, 4 or 5 heteroatom(s) independently selected from the group consisting of oxygen, nitrogen and sulphur as ring member(s);
the above-mentioned heterocycloaliphatic groups can in each case optionally have 1, 2 or 3 additional heteroatom(s) independently selected from the group consisting of oxygen, nitrogen and sulphur as ring member(s);
the rings of the above-mentioned mono- or polycyclic ring systems can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl;
the rings of the above-mentioned mono- or polycyclic ring systems each have 5, 6 or 7 members and can in each case optionally have 1, 2, 3, 4 or 5 heteroatom(s) as ring member(s) which are independently selected from the group consisting of oxygen, nitrogen and sulphur;
the above-mentioned aryl or heteroaryl groups can in each case optionally be substituted with 1, 2, 3, 4 or 5 substituents independently selected from the group consisting of F, Cl, Br, I, —CN, —CF₃, —SF₅, —OH, —O—C₁₋₅ alkyl, —NH₂, —NO₂, —O—CF₃, —S—CF₃, —SH, —S—C₁₋₅ alkyl, —C₁₋₅ alkyl, —C(═O)—OH, —C(═O)—O—C₁₋₅ alkyl, —O—C(═O)—C₁₋₅ alkyl, —NH—C₁₋₅ alkyl, —N(C₁₋₅ alkyl)₂, —NH—C(═O)—O—C₁₋₅ alkyl, —C(═O)—H, —C(═O)—C₁₋₅ alkyl, —C(═O)—NH₂, —C(═O)—NH—C₁₋₅ alkyl, C(═O)—N—(C₁₋₅ alkyl)₂, —S(═O)₂—C₁₋₅ alkyl, —S(═O)₂ phenyl, —NH—S(═O)₂—C₁₋₅ alkyl, —S(═O)₂—NH—C₁₋₅ alkyl, cyclohexyl, cyclopentyl, pyridinyl, pyridazinyl, —(CH₂)-benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)-benzo[b]furanyl and benzyl can be substituted with 1, 2, 3, 4 or 5 substituents independently selected from the group consisting of F, Cl, Br, —OH, —CF₃, —SF₅, —CN, —NO₂, —C₁₋₅ alkyl, —O—C₁₋₅ alkyl, —O—CF₃, —S—CF₃, phenyl and —O-benzyl,
the above-mentioned heteroaryl groups can in each case optionally have 1, 2, 3, 4 or 5 heteroatom(s) independently selected from the group consisting of oxygen, nitrogen and sulphur as ring member(s); and
the above-mentioned C₁₋₅ alkylene groups can in each case optionally be substituted with 1, 2, 3, 4 or 5 substituents independently selected from the group consisting of F, Cl, Br, —OH, —SH, —NH₂, —CN and NO₂.

67. A method according to claim 64, wherein R² represents
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl and (1,2,3,4)-tetrahydroquinazolinyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl; or
—(CHR³⁰)—R³³; —(CHR³⁰)—(CHR³¹)—R³³; —(CHR³⁰)—(CHR³¹)—O—R³³; —(CHR³⁰) (CHR³¹)—(CHR³²)—R³³; —(CHR³⁰)—(CHR³¹)—S— (CHR³²)—R³³; —(CHR³⁰)— (CHR³¹)—(CHR³²)—N(CH₃)—R³³.

68. A method according to claim 64, wherein R³ represents hydrogen.

69. A method according to claim 64, wherein R² and R³ together with the nitrogen atom to which they are bound form a ring selected from the group consisting of pyrrolidinyl, piperidinyl, (1,3,4,5)-tetrahydropyrido[4,3-b]indolyl, (3,4)-dihydro-1H-isoquinolinyl, (1,3,4,9)-tetrahydro-[b]-carbolinyl, imidazolidinyl, (1,3)-thiazolidinyl, piperazinyl, morpholinyl, azepanyl, diazepanyl and thiomorpholinyl, wherein the ring can in each case be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₆, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)-benzo[b]furanyl and benzyl with can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl.

70. A method according to claim 64, wherein R⁶ represents
a group selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl, 2-methyl-1-propenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)—benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl; —(CHR³⁵)—C(═O)—R²⁴ or —(CHR³⁵)—(CH₂)—C(═O)—R²⁴;
—(CHR³⁶)—C(═O)—O—R²⁵ or —(CHR³⁶)—(CH₂)—C(═O)—O—R²⁵; or
—(CR³⁷R³⁸)—R⁴¹, —(CR³⁷R³⁸)—(CHR³⁹)—R⁴¹, —(CR³⁷R³⁸)— (CHR³⁹)—O—R⁴¹, —
—(CR³⁷R³⁸)—(CHR³⁹)—(CHR⁴⁰)—R⁴¹, —(CR³⁷R³⁸)—(CHR³⁹)—(CHR⁴⁰)—O—R⁴¹, —(CR³⁷R³⁸)—(CHR³⁹)—(CHR⁴⁰)—N(CH₃)—R⁴¹ or —(CR³⁷R³⁸)—(CHR³⁹)—(CHR⁴⁰)—N(C₂H₅)—R⁴¹.

71. A method according to claim 64, wherein R⁷ represents hydrogen.

72. A method according to claim 64, wherein R²⁴ and R²⁵ each independently represent
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl, wherein the group can in each case be substituted with 1 or 2 substituents independently selected from the group consisting of —C(═O)—O—CH₃, —C(═O)—O—C₂H₅, —C(═O)—O—C(CH₃)₃, —C(═O)—CH₃, —C(═O)—C₂H₅, —C(═O)—C(CH₃)₃, —C(═O)—NH—CH₃, —C(═O)—NH—C₂H₅, —C(═O)—N— (CH₃)₂, —C(═O)—N— (C₂H₅)₂, —S(═O)₂—CH₃, —S(═O)₂—C₂H₅, —S(═O)₂ phenyl, pyridinyl, phenyl and benzyl, wherein in each case the cyclic portion of the groups pyridinyl, —S(═O)₂ phenyl, phenyl and benzyl can be substituted with 1, 2, 3, 4 or 5 substituents independently selected from the group consisting of F, Cl, Br, methyl, ethyl, n-propyl, isopropyl, n-butyl, sec-butyl, isobutyl, tert-butyl, —O—CH₃ and —O—C₂H₅;
a group selected from the group consisting of phenyl, naphthyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl and pyrimidinyl, wherein the group can in each case optionally be substituted with 1, 2, 3, 4 or 5 substituents independently selected from the group consisting of F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl, or —(CH₂)—R⁴² or —(CH₂)—(CH₂)—R⁴².

73. A method according to claim 64, wherein R³⁰, R³¹, R³², R³⁵, R³⁶, R³⁷, R³⁸, R³⁹ and R⁴⁰ each independently represent
hydrogen;
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl; or
a phenyl group which can be substituted with 1, 2, 3, 4 or 5 substituents independently selected from the group consisting of F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₆, —O—C(═O)—C(CH₃)₃, —N(CH₃)₂, —N(C₂H₅)₂, —NH—CH₃, —NH—C₂H₆, —C(═O)—CH₃, —C(═O)—C₂H₅, —C(═O)—C(CH₃)₃, cyclohexyl, cyclopentyl, —O-phenyl, —O-benzyl and phenyl.

74. A method according to claim 64, wherein R³³ and R⁴¹ each independently represent
a group selected from the group consisting of methyl, ethyl, n-propyl, isopropyl, n-butyl, sec-butyl, isobutyl, tert-butyl, n-pentyl, sec-pentyl, —(CH₂)—(CH₂)—(C(CH₃)₃), n-hexyl, n-heptyl, n-octyl, —(CH₂)—(CH)(C₂H₅)—(CH₂)—(CH₂)—(CH₂)—(CH₃), vinyl, 1-propenyl, 2-propenyl, 1-butenyl, 2-butenyl, 3-butenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl, Br, I, —CN, —NO₂, —OH, —SH and —NH₂;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl and (1,2,3,4)-tetrahydroquinazolinyl, wherein the group can in each case optionally be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)—benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl; or
a group selected from the group consisting of phenyl, naphthyl, (1,3)-benzodioxolyl, (1,4)-benzodioxanyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl and benzothiazolyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl.

75. A method according to claim 64, wherein R³⁴ and R⁴² each independently represent a group selected from the group consisting of phenyl, naphthyl, thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl and pyrimidinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃.

76. A method according to claim 64, wherein
R¹ represents a —C(═O)—NR⁶R⁷ group;
R² represents
a group selected from the group consisting of methyl, ethyl, n-propyl, isopropyl, n-butyl, sec-butyl, isobutyl, tert-butyl, n-pentyl, sec-pentyl n-hexyl and n-heptyl;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl and thiomorpholinyl, wherein the group can in each case be substituted with 1 or 2 substituents independently selected from the group consisting of pyridinyl, pyridazinyl, phenyl and benzyl, wherein in each case the cyclic portion of the groups pyridinyl, pyridazinyl, phenyl and benzyl can be substituted with 1, 2, 3, 4 or 5 substituents independently selected from the group consisting of —CF₃, F, Cl, Br, methyl, ethyl, n-propyl, isopropyl, n-butyl, sec-butyl, isobutyl, tert-butyl, —O—CH₃ and —O—C₂H₅;
a group selected from the group consisting of phenyl, naphthyl, thiophenyl, furanyl, pyrrolyl, pyrazolyl, pyrazinyl and pyridinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —S(═O)₂—CH₃, —S(═O)₂—C₂H₅, —NH—S(═O)₂—CH₃, —NH—S(═O)₂—C₂H₅, —S(═O)₂—NH—CH₃ and —S(═O)₂—NH—C₂H₅; or
—(CHR³⁰)—R³³; —(CHR³⁰)—(CHR³¹)—R³³ or —(CHR³⁰)—(CHR³¹)—(CHR³²)—R³³;
R³ represents hydrogen; or
R² and R³ together with the nitrogen atom to which they are bound form a ring selected from the group consisting of pyrrolidinyl, piperidinyl, piperazinyl, 3-methylpiperazinyl, 2-methylpiperazinyl, (3,5)-dimethylpiperazinyl, (2,6)-dimethylpiperazinyl, morpholinyl and thiomorpholinyl, wherein the ring optionally may be substituted with 1 or 2 substituents independently selected from the group consisting of —C(═O)—O—CH₃, —C(═O)—O—C₂H₅, —C(═O)—O—C(CH₃)₃, —C(═O)—CH₃, —C(═O)—C₂H₅, —C(═O)—C(CH₃)₃, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, pyridinyl, pyridazinyl, phenyl and benzyl, wherein in each case the cyclic portion of the groups pyridinyl, —S(═O)₂ phenyl, pyridazinyl, phenyl and benzyl can be substituted with 1, 2, 3, 4 or 5 substituents independently selected from the group consisting of —CF₃, F, Cl, Br, methyl, ethyl, n-propyl, isopropyl, n-butyl, sec-butyl, isobutyl, tert-butyl, —O—CH₃ and —O—C₂H₅;
R⁶ represents
a group selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl, 2-methyl-1-propenyl, ethinyl, 1-propinyl, 2-propinyl, 1-butinyl, 2-butinyl and 3-butinyl, wherein the group can in each case optionally be substituted with 1, 2, 3, 4 or 5 substituents independently selected from the group consisting of F, Cl and Br;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl and indenyl;
a group selected from the group consisting of phenyl, naphthyl, thiophenyl, furanyl, pyrrolyl, pyrazolyl, pyranyl, triazolyl, pyridinyl, imidazolyl, thiazolyl, oxazolyl, isoxazolyl, pyridazinyl, pyrazinyl and pyrimidinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —S(═O)₂ phenyl, —NH—S(═O)₂—CH₃, —NH—S(═O)₂—C₂H₅, —S(═O)₂—NH—CH₃, —S(═O)₂—NH—C₂H₅, cyclohexyl, cyclopentyl, pyridinyl, pyridazinyl, —(CH₂)— benzo[b]furanyl, —O-phenyl, —O-benzyl, phenyl and benzyl, wherein in each case the cyclic portion of the groups pyridinyl, cyclopentyl, cyclohexyl, pyridazinyl, —S(═O)₂ phenyl, —O-phenyl, —O-benzyl, phenyl, —(CH₂)— benzo[b]furanyl and benzyl can be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
—(CHR³⁶)—C(═O)—O—R²⁵ or —(CHR³⁶)—(CH₂)—C(═O)—O—R²⁵; or
—(CR³⁷R³⁸)—R⁴¹, —(CR³⁷R³⁸)—(CHR³⁹)—R⁴¹, —(CR³⁷R³⁸)— (CHR³⁹)—O—R⁴¹, —(CR³⁷R³⁸)—(CHR³⁹)—(CHR⁴⁰)—R⁴¹, —(CR³⁷R³⁸)—(CHR³⁹)—(CHR⁴⁰)—O—R⁴¹, —(CR³⁷R³⁸)—(CHR³⁹)—(CHR⁴⁰)—N(CH₃)—R⁴¹ or —(CR³⁷R³⁸)—(CHR³⁹)—(CHR⁴⁰)—N(C₂H₅)—R⁴¹;
R⁷ represents hydrogen;
R²⁵ represents a group selected from the group consisting of methyl, ethyl, n-propyl, isopropyl, n-butyl, sec-butyl, isobutyl and tert-butyl;
R³⁰, R³¹, R³², R³⁶, R³⁷, R³⁸, R³⁹ and R⁴⁰ each independently represent hydrogen;
a group selected from the group consisting of methyl, ethyl, n-propyl, isopropyl, n-butyl, sec-butyl, isobutyl and tert-butyl; or
a phenyl group which can be substituted with 1, 2, 3, 4 or 5 substituents independently selected from the group consisting of F, Cl, Br, I, —CN, —CF₃, —O—CH₃, —O—C₂H₅, —NO₂, —O—CF₃, methyl, ethyl, n-propyl, isopropyl, n-butyl, sec-butyl, isobutyl and tert-butyl;
R³³ represents a group selected from the group consisting of phenyl, naphthyl, thiophenyl, furanyl, pyrrolyl, pyrazolyl, pyrazinyl and pyridinyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —S(═O)₂—CH₃, —S(═O)₂—C₂H₅, —NH—S(═O)₂—CH₃, —NH—S(═O)₂—C₂H₅, —S(═O)₂—NH—CH₃ and —S(═O)₂—NH—C₂H₅; and
R⁴¹ represents
a group selected from the group consisting of methyl, ethyl, n-propyl, isopropyl, n-butyl, sec-butyl, isobutyl and tert-butyl;
a group selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl and dithiolanyl; or
a group selected from the group consisting of phenyl, naphthyl, (1,3)-benzodioxolyl, (1,4)-benzodioxanyl, thiophenyl and furanyl, wherein the group can in each case optionally be substituted with 1, 2, 3, 4 or 5 substituents independently selected from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅ and —C(═O)—C(CH₃)₃.

77. A method according to claim 64, wherein
R¹ represents a —C(═O)—NR⁶R⁷ group;
R² represents
a group selected from the group consisting of phenyl, naphthyl and pyridinyl, wherein the group can in each case be substituted with 1, 2 or 3 substituents independently selected from the group consisting of F, Cl, Br, —OH, —O—CH₃, —O—C₂H₅, —NH—S(═O)₂—CH₃ and —NH—S(═O)₂—C₂H₅;
a piperazinyl group which on a nitrogen atom can be substituted with a substituent selected from the group consisting of pyridinyl, pyridazinyl, phenyl and benzyl, wherein in each case the cyclic portion of the substituents pyridinyl, pyridazinyl, phenyl and benzyl can be substituted with 1, 2 or 3 substituents independently selected from the group consisting of —CF₃, F, Cl, Br, methyl, ethyl, n-propyl, isopropyl, n-butyl, sec-butyl, isobutyl and tert-butyl; or
—(CHR³⁰)—R³³; —(CHR³⁰)—(CHR³¹)—R³³ or —(CHR³⁰)—(CHR³¹)—(CHR³²)—R³³;
R³ represents hydrogen; or
R² and R³ together with the nitrogen atom to which they are bound form a ring selected from the group consisting of 3-methylpiperazinyl, 2-methylpiperazinyl, (3,5)-dimethylpiperazinyl, (2,6)-dimethylpiperazinyl and piperazinyl, which can be substituted at a nitrogen atom with a substituent selected from the group consisting of pyridinyl, pyridazinyl, phenyl and benzyl, wherein in each case the cyclic portion of the substituents pyridinyl, pyridazinyl, phenyl and benzyl can be substituted with 1, 2 or 3 substituents independently selected from the group consisting of —CF₃, F, Cl, Br, methyl, ethyl, n-propyl, isopropyl, n-butyl, sec-butyl, isobutyl and tert-butyl;
R⁶ represents
a group selected from the group consisting of methyl, ethyl, n-propyl, isopropyl, n-butyl, sec-butyl, isobutyl, tert-butyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, vinyl, 1-propenyl, 2-propenyl, 1-butenyl, 2-butenyl, 3-butenyl and 2-methyl-1-propenyl, wherein the group can in each case optionally be substituted with 1, 2, 3, 4 or 5 substituents independently selected from the group consisting of F, Cl and Br;
a group selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl and adamantyl;
a group selected from the group consisting of phenyl, naphthyl and pyridinyl, wherein the group can in each case optionally be substituted with 1, 2, 3, 4 or 5 substituents independently selected from the group consisting of —SF₅, F, Cl, Br, I, —CN, —CF₃, —O—CH₃, —O—C₂H₅, —NO₂, —O—CF₃, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —C(═O)—CH₃, —C(═O)—C₂H₅, —C(═O)—C(CH₃)₃, cyclohexyl, cyclopentyl, —O-phenyl, —O-benzyl and phenyl, wherein in each case the cyclic portion of the groups cyclopentyl, cyclohexyl, —O-phenyl, —O-benzyl and phenyl can be substituted with 1, 2, 3, 4 or 5 substituents independently selected from the group consisting of F, Cl, Br, methyl, ethyl, n-propyl, isopropyl, n-butyl, sec-butyl, isobutyl and tert-butyl;
—(CHR³⁶)—C(═O)—O—R²⁵ or —(CHR³⁶)—(CH₂)—C(═O)—O—R²⁵; or
—(CR³⁷R³⁸)—R⁴¹, —(CR³⁷R³⁸)—(CHR³⁹)—R⁴¹, —(CR³⁷R³⁸)— (CHR³⁹)—O—R⁴¹, —(CR³⁷R³⁸)—(CHR³⁹)—(CHR⁴⁰)—R⁴¹, —(CR³⁷R³⁸)—(CHR³⁹)—(CHR⁴⁰)—O—R⁴¹, —(CR³⁷R³⁸)—(CHR³⁹)—(CHR⁴⁰)—N(CH₃)—R⁴¹ or —(CR³⁷R³⁸)— (CHR³⁹)—(CHR⁴⁰)—N(C₂H₅)—R⁴¹;
R⁷ represents hydrogen;
R²⁵ represents a group selected from the group consisting of methyl, ethyl, n-propyl, isopropyl, n-butyl, sec-butyl, isobutyl and tert-butyl;
R³⁰, R³¹, R³², R³⁹ and R⁴⁰ each represent hydrogen;
R³³ represents a group selected from the group consisting of phenyl, naphthyl, thiophenyl and furanyl, wherein the group can in each case optionally be substituted with 1, 2, 3, 4 or 5 substituents independently selected from the group consisting of —CF₃, F, Cl, Br, —OH, —O—CH₃, —O—C₂H₅, methyl, ethyl, n-propyl, isopropyl, n-butyl, sec-butyl, isobutyl, tert-butyl, —NH—S(═O)₂—CH₃ and —NH—S(═O)₂—C₂H₅;
R³⁶ represents hydrogen or a group selected from the group consisting of methyl, ethyl, n-propyl, isopropyl, n-butyl, sec-butyl, isobutyl and tert-butyl;
R³⁷ and R³⁸, independently of one another, each represent
hydrogen;
a group selected from the group consisting of methyl, ethyl, n-propyl, isopropyl, n-butyl, sec-butyl, isobutyl and tert-butyl; or
a phenyl group;
R⁴¹ represents
a group selected from the group consisting of methyl, ethyl, n-propyl, isopropyl, n-butyl, sec-butyl, isobutyl and tert-butyl;
a group selected from the group consisting of tetrahydrofuranyl, tetrahydrothiophenyl, pyrrolidinyl, morpholinyl, piperidinyl and piperazinyl; or
a group selected from the group consisting of phenyl, naphthyl, (1,3)-benzodioxolyl, (1,4)-benzodioxanyl, thiophenyl and furanyl, wherein the group can in each case optionally be substituted with 1, 2, 3, 4 or 5 substituents independently selected from the group consisting of F, Cl, Br, —OH, —O—CH₃, —O—C₂H₅, methyl, ethyl, n-propyl, isopropyl, n-butyl, sec-butyl, isobutyl and tert-butyl.

78. A method according to claim 64, wherein said compound is selected from the group consisting of:
[1] 3-[4-(3-chloropyridin-2-yl)piperazine-1-carbonyl]-1-oxa-2,8-diazaspiro[4.5]dec-2-ene-8-carboxylic acid-(2-fluorophenyl)amide
[2] 1-oxa-2,8-diazaspiro[4.5]dec-2-ene-3,8-dicarboxylic acid-3-[(5-chloropyridin-2-yl)amide]-8-[(2-fluorophenyl)amide]
[3] 1-oxa-2,8-diazaspiro[4.5]dec-2-ene-3,8-dicarboxylic acid-3-(4-chlorobenzylamide)-8-[(3-fluorophenyl)amide]
[4] 3-[4-(3-chloropyridin-2-yl)piperazine-1-carbonyl]-1-oxa-2,8-diazaspiro[4.5]dec-2-ene-8-carboxylic acid-(3-fluorophenyl)amide
[5] 1-oxa-2,8-diazaspiro[4.5]dec-2-ene-3,8-dicarboxylic acid-3-[(5-chloropyridin-2-yl)amide]-8-[(3-fluorophenyl)amide]
[6] 1-oxa-2,8-diazaspiro[4.5]dec-2-ene-3,8-dicarboxylic acid-8-[(2-chlorophenyl)amide]-3-(3,4-dimethoxybenzylamide)
[7] 1-oxa-2,8-diazaspiro[4.5]dec-2-ene-3,8-dicarboxylic acid-3-(4-chlorobenzylamide)-8-[(2-chlorophenyl)amide]
[8] 3-[4-(3-chloropyridin-2-yl)piperazine-1-carbonyl]-1-oxa-2,8-diazaspiro[4.5]dec-2-ene-8-carboxylic acid-(2-chlorophenyl)amide
[9] 1-oxa-2,8-diazaspiro[4.5]dec-2-ene-3,8-dicarboxylic acid-8-[(2-chlorophenyl)amide]-3-[(5-chloropyridin-2-yl)amide]
[10] 1-oxa-2,8-diazaspiro[4.5]dec-2-ene-3,8-dicarboxylic acid-8-[(3-bromophenyl)amide]-3-(3,4-dimethoxybenzylamide)
[11] 3-[4-(3-chloropyridin-2-yl)piperazine-1-carbonyl]-1-oxa-2,8-diazaspiro[4.5]dec-2-ene-8-carboxylic acid-(3-bromophenyl)amide
[12] 1-oxa-2,8-diazaspiro[4.5]dec-2-ene-3,8-dicarboxylic acid-8-[(3-bromophenyl)amide]-3-[(5-chloropyridin-2-yl)amide]
[13] 1-oxa-2,8-diazaspiro[4.5]dec-2-ene-3,8-dicarboxylic acid-3-(3,4-dimethoxybenzylamide)-8-[(2-methoxyphenyl)amide]
[14] 1-oxa-2,8-diazaspiro[4.5]dec-2-ene-3,8-dicarboxylic acid-3-(4-chlorobenzylamide)-8-[(2-methoxyphenyl)amide]
[15] 3-[4-(3-chloropyridin-2-yl)piperazine-1-carbonyl]-1-oxa-2,8-diazaspiro[4.5]dec-2-ene-8-carboxylic acid-(2-methoxyphenyl) amide
[16] 1-oxa-2,8-diazaspiro[4.5]dec-2-ene-3,8-dicarboxylic acid-3-(3,4-dimethoxybenzylamide)-8-[(3-methoxyphenyl)amide]
[17] 3-[4-(3-chloropyridin-2-yl)piperazine-1-carbonyl]-1-oxa-2,8-diazaspiro[4.5]dec-2-ene-8-carboxylic acid-(3-methoxyphenyl)amide
[18] 1-oxa-2,8-diazaspiro[4.5]dec-2-ene-3,8-dicarboxylic acid-3-[(5-chloropyridin-2-yl)amide]-8-[(3-methoxyphenyl)amide]
[19] 3-[4-(3-chloropyridin-2-yl)piperazine-1-carbonyl]-1-oxa-2,8-diazaspiro[4.5]dec-2-ene-8-carboxylic acid-(4-phenoxyphenyl)amide
[20] 1-oxa-2,8-diazaspiro[4.5]dec-2-ene-3,8-dicarboxylic acid-8-[(2-chloro-5-trifluoromethylphenyl)amide]-3-(3,4-dimethoxybenzylamide)
[21] 1-oxa-2,8-diazaspiro[4.5]dec-2-ene-3,8-dicarboxylic acid-3-(4-chlorobenzylamide)-8-[(2-chloro-5-trifluoromethylphenyl)amide]
[22] 3-[4-(3-chloropyridin-2-yl)piperazine-1-carbonyl]-1-oxa-2,8-diazaspiro[4.5]dec-2-ene-8-carboxylic acid-(2-chloro-5-trifluoromethylphenyl)amide
[23] 1-oxa-2,8-diazaspiro[4.5]dec-2-ene-3,8-dicarboxylic acid-3-[(5-chloropyridin-2-yl)amide]-8-[(2-chloro-5-trifluoromethylphenyl)amide]
[24] 1-oxa-2,8-diazaspiro[4.5]dec-2-ene-3,8-dicarboxylic acid-8-[(4-chloro-2-trifluoromethylphenyl)amide]-3-(3,4-dimethoxybenzylamide)
[25] 3-[4-(3-chloropyridin-2-yl)piperazine-1-carbonyl]-1-oxa-2,8-diazaspiro[4.5]dec-2-ene-8-carboxylic acid-(4-chloro-2-trifluoromethylphenyl)amide
[26] 1-oxa-2,8-diazaspiro[4.5]dec-2-ene-3,8-dicarboxylic acid-3-[(5-chloropyridin-2-yl)amide]-8-[(4-chloro-2-trifluoromethylphenyl)amide]
[27] 1-oxa-2,8-diazaspiro[4.5]dec-2-ene-3,8-dicarboxylic acid-8-[(4-chloro-3-trifluoromethylphenyl)amide]-3-(3,4-dimethoxybenzylamide)
[28] 3-[4-(3-chloropyridin-2-yl)piperazine-1-carbonyl]-1-oxa-2,8-diazaspiro[4.5]dec-2-ene-8-carboxylic acid-(4-chloro-3-trifluoromethylphenyl)amide
[29] 1-oxa-2,8-diazaspiro[4.5]dec-2-ene-3,8-dicarboxylic acid-3-[(5-chloropyridin-2-yl)amide]-8-[(4-chloro-3-trifluoromethylphenyl)amide]
[30] 1-oxa-2,8-diazaspiro[4.5]dec-2-ene-3,8-dicarboxylic acid-3-[(2-tert-butyl-6-methylphenyl)amide]-8-(4-chlorobenzylamide)
[31] 3-[4-(3-chloropyridin-2-yl)piperazine-1-carbonyl]-1-oxa-2,8-diazaspiro[4.5]dec-2-ene-8-carboxylic acid-(2-tert-butyl-6-methylphenyl)amide
[32] 1-oxa-2,8-diazaspiro[4.5]dec-2-ene-3,8-dicarboxylic acid-3-(3,4-dimethoxybenzylamide)-8-[(4-trifluoromethoxyphenyl)amide]
[33] 1-oxa-2,8-diazaspiro[4.5]dec-2-ene-3,8-dicarboxylic acid-3-(4-chlorobenzylamide)-8-[(4-trifluoromethoxyphenyl)amide]
[34] 3-[4-(3-chloropyridin-2-yl)piperazine-1-carbonyl]-1-oxa-2,8-diazaspiro[4.5]dec-2-ene-8-carboxylic acid-(4-trifluoromethoxyphenyl)amide
[35] 1-oxa-2,8-diazaspiro[4.5]dec-2-ene-3,8-dicarboxylic acid-3-[(5-chloropyridin-2-yl)amide]-8-[(4-trifluoromethoxyphenyl)amide]
[36] 1-oxa-2,8-diazaspiro[4.5]dec-2-ene-3,8-dicarboxylic acid-3-(3,4-dimethoxybenzylamide)-8-(phenethylamide)
[37] 1-oxa-2,8-diazaspiro[4.5]dec-2-ene-3,8-dicarboxylic acid-3-(4-chlorobenzylamide)-8-(phenethylamide)
[38] 3-[4-(3-chloropyridin-2-yl)piperazine-1-carbonyl]-1-oxa-2,8-diazaspiro[4.5]dec-2-ene-8-carboxylic acid phenethylamide
[39] 1-oxa-2,8-diazaspiro[4.5]dec-2-ene-3,8-dicarboxylic acid-3-(3,4-dimethoxybenzylamide)-8-phenylamide
[40] 3-[4-(3-chloropyridin-2-yl)piperazine-1-carbonyl]-1-oxa-2,8-diazaspiro[4.5]dec-2-ene-8-carboxylic acid phenylamide
[41] 1-oxa-2,8-diazaspiro[4.5]dec-2-ene-3,8-dicarboxylic acid-3-[(5-chloropyridin-2-yl)amide]-8-phenylamide
[42] 1-oxa-2,8-diazaspiro[4.5]dec-2-ene-3,8-dicarboxylic acid-3-(4-chlorobenzylamide)-8-m-tolylamide
[43] 3-[4-(3-chloropyridin-2-yl)piperazine-1-carbonyl]-1-oxa-2,8-diazaspiro[4.5]dec-2-ene-8-carboxylic acid-m-tolylamide
[44] 1-oxa-2,8-diazaspiro[4.5]dec-2-ene-3,8-dicarboxylic acid-3-(3,4-dimethoxybenzylamide)-8-[(4-fluorophenyl)amide]
[45] 3-[4-(3-chloropyridin-2-yl)piperazine-1-carbonyl]-1-oxa-2,8-diazaspiro[4.5]dec-2-ene-8-carboxylic acid-(4-fluorophenyl)amide
[46] 1-oxa-2,8-diazaspiro[4.5]dec-2-ene-3,8-dicarboxylic acid-8-[(3-chlorophenyl)amide]-3-(3,4-dimethoxybenzylamide)
[47] 3-[4-(3-chloropyridin-2-yl)piperazine-1-carbonyl]-1-oxa-2,8-diazaspiro[4.5]dec-2-ene-8-carboxylic acid-(3-chlorophenyl)amide
[48] 1-oxa-2,8-diazaspiro[4.5]dec-2-ene-3,8-dicarboxylic acid-8-[(3-chlorophenyl)amide]-3-[(5-chloropyridin-2-yl)amide]
[49] 1-oxa-2,8-diazaspiro[4.5]dec-2-ene-3,8-dicarboxylic acid-8-[(4-chlorophenyl)amide]-3-(3,4-dimethoxybenzylamide)
[50] 1-oxa-2,8-diazaspiro[4.5]dec-2-ene-3,8-dicarboxylic acid-3-(4-chlorobenzylamide)-8-[(4-chlorophenyl)amide]
[51] 3-[4-(3-chloropyridin-2-yl)piperazine-1-carbonyl]-1-oxa-2,8-diazaspiro[4.5]dec-2-ene-8-carboxylic acid-(4-chlorophenyl)amide
[52] 3-[4-(3-chloropyridin-2-yl)piperazine-1-carbonyl]-1-oxa-2,8-diazaspiro[4.5]dec-2-ene-8-carboxylic acid-(4-methoxyphenyl)amide
[53] 1-oxa-2,8-diazaspiro[4.5]dec-2-ene-3,8-dicarboxylic acid-3-(3,4-dimethoxybenzylamide)-8-[(2-methylsulphanylphenyl)amide]
[54] 3-[4-(3-chloropyridin-2-yl)piperazine-1-carbonyl]-1-oxa-2,8-diazaspiro[4.5]dec-2-ene-8-carboxylic acid-(2-methylsulphanylphenyl)amide
[55] 1-oxa-2,8-diazaspiro[4.5]dec-2-ene-3,8-dicarboxylic acid-3-(3,4-dimethoxybenzylamide)-8-[(3-methylsulphanylphenyl)amide]
[56] 3-[4-(3-chloropyridin-2-yl)piperazine-1-carbonyl]-1-oxa-2,8-diazaspiro[4.5]dec-2-ene-8-carboxylic acid-(3-methylsulphanylphenyl)amide
[57] 1-oxa-2,8-diazaspiro[4.5]dec-2-ene-3,8-dicarboxylic acid-3-(3,4-dimethoxybenzylamide)-8-[(4-methylsulphanylphenyl)amide]
[58] 3-[4-(3-chloropyridin-2-yl)piperazine-1-carbonyl]-1-oxa-2,8-diazaspiro[4.5]dec-2-ene-8-carboxylic acid-(4-methylsulphanylphenyl)amide
[59] 1-oxa-2,8-diazaspiro[4.5]dec-2-ene-3,8-dicarboxylic acid-3-[(5-chloropyridin-2-yl)amide]-8-[(4-methylsulphanylphenyl)amide]
[60] 1-oxa-2,8-diazaspiro[4.5]dec-2-ene-3,8-dicarboxylic acid-3-(3,4-dimethoxybenzylamide)-8-[(2-isopropylphenyl)amide]
[61] 3-[4-(3-chloropyridin-2-yl)piperazine-1-carbonyl]-1-oxa-2,8-diazaspiro[4.5]dec-2-ene-8-carboxylic acid-(2-isopropylphenyl)amide
[62] 1-oxa-2,8-diazaspiro[4.5]dec-2-ene-3,8-dicarboxylic acid-3-[(5-chloropyridin-2-yl)amide]-8-[(2-isopropylphenyl)amide]
[63] 3-[4-(3-chloropyridin-2-yl)piperazine-1-carbonyl]-1-oxa-2,8-diazaspiro[4.5]dec-2-ene-8-carboxylic acid-(4-isopropylphenyl)amide
[64] 1-oxa-2,8-diazaspiro[4.5]dec-2-ene-3,8-dicarboxylic acid-3-(3,4-dimethoxybenzylamide)-8-[(2-trifluoromethylphenyl)amide]
[65] 3-[4-(3-chloropyridin-2-yl)piperazine-1-carbonyl]-1-oxa-2,8-diazaspiro[4.5]dec-2-ene-8-carboxylic acid-(2-trifluoromethylphenyl)amide
[66] 1-oxa-2,8-diazaspiro[4.5]dec-2-ene-3,8-dicarboxylic acid-3-[(5-chloropyridin-2-yl)amide]-8-[(2-trifluoromethylphenyl)amide]
[67] 1-oxa-2,8-diazaspiro[4.5]dec-2-ene-3,8-dicarboxylic acid-3-(3,4-dimethoxybenzylamide)-8-[(3-trifluoromethylphenyl)amide]
[68] 3-[4-(3-chloropyridin-2-yl)piperazine-1-carbonyl]-1-oxa-2,8-diazaspiro[4.5]dec-2-ene-8-carboxylic acid-(3-trifluoromethylphenyl)amide
[69] 1-oxa-2,8-diazaspiro[4.5]dec-2-ene-3,8-dicarboxylic acid-3-[(5-chloropyridin-2-yl)amide]-8-[(3-trifluoromethylphenyl)amide]
[70] 1-oxa-2,8-diazaspiro[4.5]dec-2-ene-3,8-dicarboxylic acid-3-(3,4-dimethoxybenzylamide)-8-[(4-trifluoromethylphenyl)amide]
[71] 3-[4-(3-chloropyridin-2-yl)piperazine-1-carbonyl]-1-oxa-2,8-diazaspiro[4.5]dec-2-ene-8-carboxylic acid-(4-trifluoromethylphenyl)amide
[72] 1-oxa-2,8-diazaspiro[4.5]dec-2-ene-3,8-dicarboxylic acid-8-cyclohexylamide-3-(3,4-dimethoxybenzylamide)
[73] 1-oxa-2,8-diazaspiro[4.5]dec-2-ene-3,8-dicarboxylic acid-3-(4-chlorobenzylamide)-8-cyclohexylamide
[74] 1-oxa-2,8-diazaspiro[4.5]dec-2-ene-3,8-dicarboxylic acid-8-benzylamide-3-(3,4-dimethoxybenzylamide)
[75] 1-oxa-2,8-diazaspiro[4.5]dec-2-ene-3,8-dicarboxylic acid-3-(3,4-dimethoxybenzylamide)-8-o-tolylamide
[76] 1-oxa-2,8-diazaspiro[4.5]dec-2-ene-3,8-dicarboxylic acid-3-(4-chlorobenzylamide)-8-o-tolylamide
[77] 1-oxa-2,8-diazaspiro[4.5]dec-2-ene-3,8-dicarboxylic acid-3-[(5-chloropyridin-2-yl)amide]-8-o-tolylamide
[78] 1-oxa-2,8-diazaspiro[4.5]dec-2-ene-3,8-dicarboxylic acid-3-(3,4-dimethoxybenzylamide)-8-[(2-ethylphenyl)amide]
[79] 3-[4-(3-chloropyridin-2-yl)piperazine-1-carbonyl]-1-oxa-2,8-diazaspiro[4.5]dec-2-ene-8-carboxylic acid-(2-ethylphenyl)amide
[80] 1-oxa-2,8-diazaspiro[4.5]dec-2-ene-3,8-dicarboxylic acid-3-[(5-chloropyridin-2-yl)amide]-8-[(2-ethylphenyl)amide]
[81] 1-oxa-2,8-diazaspiro[4.5]dec-2-ene-3,8-dicarboxylic acid-3-(4-chlorobenzylamide)-8-[(4-ethylphenyl)amide]
[82] 1-oxa-2,8-diazaspiro[4.5]dec-2-ene-3,8-dicarboxylic acid-3-[(5-chloropyridin-2-yl)amide]-8-[(4-ethylphenyl)amide]
[83] 1-oxa-2,8-diazaspiro[4.5]dec-2-ene-3,8-dicarboxylic acid-3-(3,4-dimethoxybenzylamide)-8-[(2-fluorophenyl)amide]
[84] 1-oxa-2,8-diazaspiro[4.5]dec-2-ene-3,8-dicarboxylic acid-3-(3,4-dimethoxybenzylamide)-8-p-tolylamide
[85] 1-oxa-2,8-diazaspiro[4.5]dec-2-ene-3,8-dicarboxylic acid-3-(4-chlorobenzylamide)-8-p-tolylamide
[86] 3-[4-(3-chloropyridin-2-yl)piperazine-1-carbonyl]-1-oxa-2,8-diazaspiro[4.5]dec-2-ene-8-carboxylic acid p-tolylamide
[87] 1-oxa-2,8-diazaspiro[4.5]dec-2-ene-3,8-dicarboxylic acid-3-[(5-chloropyridin-2-yl)amide]-8-p-tolylamide
[88] 1-oxa-2,8-diazaspiro[4.5]dec-2-ene-3,8-dicarboxylic acid-3-(4-chlorobenzylamide)-8-[(3-ethylphenyl)amide]
[89] 3-[4-(3-chloropyridin-2-yl)piperazine-1-carbonyl]-1-oxa-2,8-diazaspiro[4.5]dec-2-ene-8-carboxylic acid-(3-ethylphenyl)amide
[90] 1-oxa-2,8-diazaspiro[4.5]dec-2-ene-3,8-dicarboxylic acid-3-[(5-chloropyridin-2-yl)amide]-8-[(3-ethylphenyl)amide]
[91] 1-oxa-2,8-diazaspiro[4.5]dec-2-ene-3,8-dicarboxylic acid-3-(3,4-dimethoxybenzylamide)-8-[(2-propylphenyl)amide]
[92] 1-oxa-2,8-diazaspiro[4.5]dec-2-ene-3,8-dicarboxylic acid-3-(4-chlorobenzylamide)-8-[(2-propylphenyl)amide]
[93] 1-oxa-2,8-diazaspiro[4.5]dec-2-ene-3,8-dicarboxylic acid-3-(4-chlorobenzylamide)-8-[(2-propylphenyl)amide]
[94] 3-[4-(3-chloropyridin-2-yl)piperazine-1-carbonyl]-1-oxa-2,8-diazaspiro[4.5]dec-2-ene-8-carboxylic acid-(2-propylphenyl)amide
[95] 1-oxa-2,8-diazaspiro[4.5]dec-2-ene-3,8-dicarboxylic acid-3-[(5-chloropyridin-2-yl)amide]-8-[(2-propylphenyl)amide]
[96] 1-oxa-2,8-diazaspiro[4.5]dec-2-ene-3,8-dicarboxylic acid-8-[(2-bromophenyl)amide]-3-(3,4-dimethoxybenzylamide)
[97] 1-oxa-2,8-diazaspiro[4.5]dec-2-ene-3,8-dicarboxylic acid-8-[(2-bromophenyl)amide]-3-(4-chlorobenzylamide)
[98] 3-[4-(3-chloropyridin-2-yl)piperazine-1-carbonyl]-1-oxa-2,8-diazaspiro[4.5]dec-2-ene-8-carboxylic acid-(2-bromophenyl)amide
[99] 3-[4-(3-chloropyridin-2-yl)piperazine-1-carbonyl]-1-oxa-2,8-diazaspiro[4.5]dec-2-ene-8-carboxylic acid-(4-bromophenyl)amide
[100] 3-[4-(3-chloropyridin-2-yl)piperazine-1-carbonyl]-1-oxa-2,8-diazaspiro[4.5]dec-2-ene-8-carboxylic acid-biphenyl-4-ylamide
[101] 1-oxa-2,8-diazaspiro[4.5]dec-2-ene-3,8-dicarboxylic acid-3-(3,4-dimethoxybenzylamide)-8-[(2-phenoxyphenyl)amide]
[102] 3-[4-(3-chloropyridin-2-yl)piperazine-1-carbonyl]-1-oxa-2,8-diazaspiro[4.5]dec-2-ene-8-carboxylic acid-(2-phenoxyphenyl)amide
[103] 1-oxa-2,8-diazaspiro[4.5]dec-2-ene-3,8-dicarboxylic acid-3-[(5-chloropyridin-2-yl)amide]-8-[(2-phenoxyphenyl)amide]
[104] 1-oxa-2,8-diazaspiro[4.5]dec-2-ene-3,8-dicarboxylic acid-3-(4-chlorobenzylamide)-8-[(2-trifluoromethoxyphenyl)amide]
[105] 3-[4-(3-chloropyridin-2-yl)piperazine-1-carbonyl]-1-oxa-2,8-diazaspiro[4.5]dec-2-ene-8-carboxylic acid-(2-trifluoromethoxyphenyl)amide
[106] 1-oxa-2,8-diazaspiro[4.5]dec-2-ene-3,8-dicarboxylic acid-3-[(5-chloropyridin-2-yl)amide]-8-[(2-trifluoromethoxyphenyl)amide]
[107] 1-oxa-2,8-diazaspiro[4.5]dec-2-ene-3,8-dicarboxylic acid-3-(3,4-dimethoxybenzylamide)-8-(4-methylbenzylamide)
[108] 1-oxa-2,8-diazaspiro[4.5]dec-2-ene-3,8-dicarboxylic acid-3-(4-chlorobenzylamide)-8-(4-methylbenzylamide)
[109] 3-[4-(3-chloropyridin-2-yl)piperazine-1-carbonyl]-1-oxa-2,8-diazaspiro[4.5]dec-2-ene-8-carboxylic acid-4-methylbenzylamide
[110] 1-oxa-2,8-diazaspiro[4.5]dec-2-ene-3,8-dicarboxylic acid-3-[(5-chloropyridin-2-yl)amide]-8-(4-methylbenzylamide)
[111] 1-oxa-2,8-diazaspiro[4.5]dec-2-ene-3,8-dicarboxylic acid-3-(3,4-dimethoxybenzylamide)-8-(4-methoxybenzylamide)
[112] 1-oxa-2,8-diazaspiro[4.5]dec-2-ene-3,8-dicarboxylic acid-3-(4-chlorobenzylamide)-8-(4-methoxybenzylamide)
[113] 3-[4-(3-chloropyridin-2-yl)piperazine-1-carbonyl]-1-oxa-2,8-diazaspiro[4.5]dec-2-ene-8-carboxylic acid-4-methoxybenzylamide
[114] 1-oxa-2,8-diazaspiro[4.5]dec-2-ene-3,8-dicarboxylic acid-8-[(4-tert-butylphenyl)amide]-3-(3,4-dimethoxybenzylamide)
[115] 1-oxa-2,8-diazaspiro[4.5]dec-2-ene-3,8-dicarboxylic acid-8-[(4-tert-butylphenyl)amide]-3-(4-chlorobenzylamide)
[116] 3-[4-(3-chloropyridin-2-yl)piperazine-1-carbonyl]-1-oxa-2,8-diazaspiro[4.5]dec-2-ene-8-carboxylic acid-(4-tert-butylphenyl)amide
[117] 1-oxa-2,8-diazaspiro[4.5]dec-2-ene-3,8-dicarboxylic acid-8-[(4-tert-butylphenyl)amide]-3-[(5-chloropyridin-2-yl)amide]
[118] 8-(2-methoxyphenylthiocarbamoyl)-1-oxa-2,8-diazaspiro[4.5]dec-2-ene-3-carboxylic acid-3,4-dimethoxybenzylamide
[119] 3-[4-(3-chloropyridin-2-yl)piperazine-1-carbonyl]-1-oxa-2,8-diazaspiro[4.5]dec-2-ene-8-thiocarboxylic acid-(2-methoxyphenyl)amide
[120] 8-(cyclohexylmethylthiocarbamoyl)-1-oxa-2,8-diazaspiro[4.5]dec-2-ene-3-carboxylic acid-3,4-dimethoxybenzylamide
[121] 8-(cyclohexylmethylthiocarbamoyl)-1-oxa-2,8-diazaspiro[4.5]dec-2-ene-3-carboxylic acid-4-chlorobenzylamide
[122] 3-[4-(3-chloropyridin-2-yl)piperazine-1-carbonyl]-1-oxa-2,8-diazaspiro[4.5]dec-2-ene-8-thiocarboxylic acid cyclohexylmethylamide
[123] 8 -cyclooctylthiocarbamoyl-1-oxa-2,8-diazaspiro[4.5]dec-2-ene-3-carboxylic acid-3,4-dimethoxybenzylamide
[124] 8 -cyclooctylthiocarbamoyl-1-oxa-2,8-diazaspiro[4.5]dec-2-ene-3-carboxylic acid-4-chlorobenzylamide
[125] 8-(3-morpholin-4-yl-propylthiocarbamoyl)-1-oxa-2,8-diazaspiro[4.5]dec-2-ene-3-carboxylic acid-4-chlorobenzylamide
[126] 8-(3-morpholin-4-yl-propylthiocarbamoyl)-1-oxa-2,8-diazaspiro[4.5]dec-2-ene-3-carboxylic acid-(5-chloropyridin-2-yl)amide
[127] 8-p-tolylthiocarbamoyl-1-oxa-2,8-diazaspiro[4.5]dec-2-ene-3-carboxylic acid-4-chlorobenzylamide
[128] 8-phenethylthiocarbamoyl-1-oxa-2,8-diazaspiro[4.5]dec-2-ene-3-carboxylic acid-3,4-dimethoxybenzylamide
[129] 8-phenethylthiocarbamoyl-1-oxa-2,8-diazaspiro[4.5]dec-2-ene-3-carboxylic acid-4-chlorobenzylamide
[130] 8-(3-phenyl-propylthiocarbamoyl)-1-oxa-2,8-diazaspiro[4.5]dec-2-ene-3-carboxylic acid-(5-chloropyridin-2-yl)amide
[131] 8-(2-fluorophenylthiocarbamoyl)-1-oxa-2,8-diazaspiro[4.5]dec-2-ene-3-carboxylic acid-3,4-dimethoxybenzylamide
[132] 8-(4-fluorophenylthiocarbamoyl)-1-oxa-2,8-diazaspiro[4.5]dec-2-ene-3-carboxylic acid-3,4-dimethoxybenzylamide
[133] 8-(4-fluorophenylthiocarbamoyl)-1-oxa-2,8-diazaspiro[4.5]dec-2-ene-3-carboxylic acid-4-chlorobenzylamide
[134] 8-(2-chlorophenylthiocarbamoyl)-1-oxa-2,8-diazaspiro[4.5]dec-2-ene-3-carboxylic acid-3,4-dimethoxybenzylamide
[135] 8-(2-chlorophenylthiocarbamoyl)-1-oxa-2,8-diazaspiro[4.5]dec-2-ene-3-carboxylic acid-4-chlorobenzylamide
[136] 8-(3-trifluoromethylphenylthiocarbamoyl)-1-oxa-2,8-diazaspiro[4.5]dec-2-ene-3-carboxylic acid-4-chlorobenzylamide
[137] 8-(naphthalen-1-ylthiocarbamoyl)-1-oxa-2,8-diazaspiro[4.5]dec-2-ene-3-carboxylic acid-3,4-dimethoxybenzylamide
[138] 8-(naphthalen-1-ylthiocarbamoyl)-1-oxa-2,8-diazaspiro[4.5]dec-2-ene-3-carboxylic acid-3,4-dimethoxybenzylamide
[139] 8 -cyclopentylthiocarbamoyl-1-oxa-2,8-diazaspiro[4.5]dec-2-ene-3-carboxylic acid-3,4-dimethoxybenzylamide
[140] 3-[4-(3-chloropyridin-2-yl)piperazine-1-carbonyl]-1-oxa-2,8-diazaspiro[4.5]dec-2-ene-8-thiocarboxylic acid cyclopentylamide
[141] 8-(2-morpholin-4-ylethylthiocarbamoyl)-1-oxa-2,8-diazaspiro[4.5]dec-2-ene-3-carboxylic acid-3,4-dimethoxybenzylamide
[142] 8-(2-morpholin-4-ylethylthiocarbamoyl)-1-oxa-2,8-diazaspiro[4.5]dec-2-ene-3-carboxylic acid-4-chlorobenzylamide
[143] 8-(3-chlorophenylthiocarbamoyl)-1-oxa-2,8-diazaspiro[4.5]dec-2-ene-3-carboxylic acid-3,4-dimethoxybenzylamide
[144] 3-[4-(3-chloropyridin-2-yl)piperazine-1-carbonyl]-1-oxa-2,8-diazaspiro[4.5]dec-2-ene-8-thiocarboxylic acid-(4-trifluoromethylphenyl)amide
[145] 8-(2-methylsulphanylphenylthiocarbamoyl)-1-oxa-2,8-diazaspiro[4.5]dec-2-ene-3-carboxylic acid-3,4-dimethoxybenzylamide
[146] 8-(2-methylsulphanylphenylthiocarbamoyl)-1-oxa-2,8-diazaspiro[4.5]dec-2-ene-3-carboxylic acid-4-chlorobenzylamide
[147] 3-[4-(3-chloropyridin-2-yl)piperazine-1-carbonyl]-1-oxa-2,8-diazaspiro[4.5]dec-2-ene-8-thiocarboxylic acid-(2-methylsulphanylphenyl)amide
[148] 8-(4-isopropylphenylthiocarbamoyl)-1-oxa-2,8-diazaspiro[4.5]dec-2-ene-3-carboxylic acid-3,4-dimethoxybenzylamide
[149] 8-(4-isopropylphenylthiocarbamoyl)-1-oxa-2,8-diazaspiro[4.5]dec-2-ene-3-carboxylic acid-4-chlorobenzylamide
[150] 8-(2-iodophenylthiocarbamoyl)-1-oxa-2,8-diazaspiro[4.5]dec-2-ene-3-carboxylic acid-3,4-dimethoxybenzylamide
[151] 8-(2-iodophenylthiocarbamoyl)-1-oxa-2,8-diazaspiro[4.5]dec-2-ene-3-carboxylic acid-4-chlorobenzylamide
[152] 8-(2-trifluoromethylphenylthiocarbamoyl)-1-oxa-2,8-diazaspiro[4.5]dec-2-ene-3-carboxylic acid-3,4-dimethoxybenzylamide
[153] 8-(2-trifluoromethylphenylthiocarbamoyl)-1-oxa-2,8-diazaspiro[4.5]dec-2-ene-3-carboxylic acid-4-chlorobenzylamide
[154] 3-[4-(3-chloropyridin-2-yl)piperazine-1-carbonyl]-1-oxa-2,8-diazaspiro[4.5]dec-2-ene-8-thiocarboxylic acid-(2-trifluoromethylphenyl)amide
[155] 8-[(benzo[1,3] dioxol-5-ylmethyl)thiocarbamoyl]-1-oxa-2,8-diazaspiro[4.5]dec-2-ene-3-carboxylic acid-3,4-dimethoxybenzylamide
[156] 8-[(benzo[1,3]dioxol-5-ylmethyl)thiocarbamoyl]-1-oxa-2,8-diazaspiro[4.5]dec-2-ene-3-carboxylic acid-4-chlorobenzylamide
[157] 3-[4-(3-chloropyridin-2-yl)piperazine-1-carbonyl]-1-oxa-2,8-diazaspiro[4.5]dec-2-ene-8-thiocarboxylic acid-(benzo[1,3] dioxol-5-ylmethyl)amide
[158] 3-[4-(3-chloropyridin-2-yl)piperazine-1-carbonyl]-1-oxa-2,8-diazaspiro[4.5]dec-2-ene-8-carboxylic acid-(3-cyanophenyl)amide
[159] 1-oxa-2,8-diazaspiro[4.5]dec-2-ene-3,8-dicarboxylic acid-3-(3,4-dimethoxybenzylamide)-8-[(2,5-dimethoxyphenyl)amide]
[160] 1-oxa-2,8-diazaspiro[4.5]dec-2-ene-3,8-dicarboxylic acid-3-(4-chlorobenzylamide)-8-[(2,5-dimethoxyphenyl)amide]
[161] 3-[4-(3-chloropyridin-2-yl)piperazine-1-carbonyl]-1-oxa-2,8-diazaspiro[4.5]dec-2-ene-8-carboxylic acid-(2,5-dimethoxyphenyl)amide
[162] 1-oxa-2,8-diazaspiro[4.5]dec-2-ene-3,8-dicarboxylic acid-3-(3,4-dimethoxybenzylamide)-8-[(2,4-dimethylphenyl)amide]
[163] 1-oxa-2,8-diazaspiro[4.5]dec-2-ene-3,8-dicarboxylic acid-3-(4-chlorobenzylamide)-8-[(2,4-dimethylphenyl)amide]
[164] 3-[4-(3-chloropyridin-2-yl)piperazine-1-carbonyl]-1-oxa-2,8-diazaspiro[4.5]dec-2-ene-8-carboxylic acid-(2,4-dimethylphenyl)amide
[165] 1-oxa-2,8-diazaspiro[4.5]dec-2-ene-3,8-dicarboxylic acid-8-butylamide-3-(3,4-dimethoxybenzylamide)
[166] 1-oxa-2,8-diazaspiro[4.5]dec-2-ene-3,8-dicarboxylic acid-8-butylamide-3-(4-chlorobenzylamide)
[167] 3-[4-(3-chloropyridin-2-yl)piperazine-1-carbonyl]-1-oxa-2,8-diazaspiro[4.5]dec-2-ene-8-carboxylic acid butylamide
[168] 1-oxa-2,8-diazaspiro[4.5]dec-2-ene-3,8-dicarboxylic acid-3-(3,4-dimethoxybenzylamide)-8-pentylamide
[169] 1-oxa-2,8-diazaspiro[4.5]dec-2-ene-3,8-dicarboxylic acid-3-(4-chlorobenzylamide)-8-pentylamide
[170] 3-[4-(3-chloropyridin-2-yl)piperazine-1-carbonyl]-1-oxa-2,8-diazaspiro[4.5]dec-2-ene-8-carboxylic acid pentylamide
[171] 1-oxa-2,8-diazaspiro[4.5]dec-2-ene-3,8-dicarboxylic acid-3-(3,4-dimethoxybenzylamide)-8-[(2-ethoxyphenyl)amide]
[172] 1-oxa-2,8-diazaspiro[4.5]dec-2-ene-3,8-dicarboxylic acid-3-(4-chlorobenzylamide)-8-[(2-ethoxyphenyl)amide]
[173] 1-oxa-2,8-diazaspiro[4.5]dec-2-ene-3,8-dicarboxylic acid-3-(3,4-dimethoxybenzylamide)-8-[(4-ethoxyphenyl)amide]
[174] 1-oxa-2,8-diazaspiro[4.5]dec-2-ene-3,8-dicarboxylic acid-8-[(2,4-difluorophenyl)amide]-3-(3,4-dimethoxybenzylamide)
[175] 1-oxa-2,8-diazaspiro[4.5]dec-2-ene-3,8-dicarboxylic acid-3-(4-chlorobenzylamide)-8-[(2,4-difluorophenyl)amide]
[176] 1-oxa-2,8-diazaspiro[4.5]dec-2-ene-3,8-dicarboxylic acid-8-[(3-chloro-2-methylphenyl)amide]-3-(3,4-dimethoxybenzylamide)
[177] 1-oxa-2,8-diazaspiro[4.5]dec-2-ene-3,8-dicarboxylic acid-3-(4-chlorobenzylamide)-8-[(3-chloro-2-methylphenyl)amide]
[178] 1-oxa-2,8-diazaspiro[4.5]dec-2-ene-3,8-dicarboxylic acid-8-[(4-chloro-2-methylphenyl)amide]-3-(3,4-dimethoxybenzylamide)
[179] 1-oxa-2,8-diazaspiro[4.5]dec-2-ene-3,8-dicarboxylic acid-3-(4-chlorobenzylamide)-8-[(4-chloro-2-methylphenyl)amide]
[180] 1-oxa-2,8-diazaspiro[4.5]dec-2-ene-3,8-dicarboxylic acid-8-[(3-chloro-4-methylphenyl)amide]-3-(3,4-dimethoxybenzylamide)
[181] 1-oxa-2,8-diazaspiro[4.5]dec-2-ene-3,8-dicarboxylic acid-3-(4-chlorobenzylamide)-8-[(3-chloro-4-methylphenyl)amide]
[182] 1-oxa-2,8-diazaspiro[4.5]dec-2-ene-3,8-dicarboxylic acid-8-[(3-chloro-4-fluorophenyl)amide]-3-(3,4-dimethoxybenzylamide)
[183] 1-oxa-2,8-diazaspiro[4.5]dec-2-ene-3,8-dicarboxylic acid-3-(4-chlorobenzylamide)-8-[(3-chloro-4-fluorophenyl)amide]
[184] 1-oxa-2,8-diazaspiro[4.5]dec-2-ene-3,8-dicarboxylic acid-8-[(2,6-diisopropylphenyl)amide]-3-(3,4-dimethoxybenzylamide)
[185] 1-oxa-2,8-diazaspiro[4.5]dec-2-ene-3,8-dicarboxylic acid-3-(4-chlorobenzylamide)-8-[(2,6-diisopropylphenyl)amide]
[186] 1-oxa-2,8-diazaspiro[4.5]dec-2-ene-3,8-dicarboxylic acid-8-[(5-chloro-2-methoxyphenyl)amide]-3-(3,4-dimethoxybenzylamide)
[187] 1-oxa-2,8-diazaspiro[4.5]dec-2-ene-3,8-dicarboxylic acid-3-(4-chlorobenzylamide)-8-[(5-chloro-2-methoxyphenyl)amide]
[188] 1-oxa-2,8-diazaspiro[4.5]dec-2-ene-3,8-dicarboxylic acid-3-(4-chlorobenzylamide)-8-[(3,4,5-trimethoxyphenyl)amide]
[189] 1-oxa-2,8-diazaspiro[4.5]dec-2-ene-3,8-dicarboxylic acid-3-(3,4-dimethoxybenzylamide)-8-[(3,5-dimethylphenyl)amide]
[190] 1-oxa-2,8-diazaspiro[4.5]dec-2-ene-3,8-dicarboxylic acid-3-(4-chlorobenzylamide)-8-[(3,5-dimethylphenyl)amide]
[191] 1-oxa-2,8-diazaspiro[4.5]dec-2-ene-3,8-dicarboxylic acid-3-(3,4-dimethoxybenzylamide)-8-[(2,6-dimethylphenyl)amide]
[192] 1-oxa-2,8-diazaspiro[4.5]dec-2-ene-3,8-dicarboxylic acid-3-(4-chlorobenzylamide)-8-[(2,6-dimethylphenyl)amide]
[193] 1-oxa-2,8-diazaspiro[4.5]dec-2-ene-3,8-dicarboxylic acid-3-(3,4-dimethoxybenzylamide)-8-[(3,4-dimethylphenyl)amide]
[194] 1-oxa-2,8-diazaspiro[4.5]dec-2-ene-3,8-dicarboxylic acid-3-(3,4-dimethoxybenzylamide)-8-[(2,5-dimethylphenyl)amide]
[195] 1-oxa-2,8-diazaspiro[4.5]dec-2-ene-3,8-dicarboxylic acid-3-(4-chlorobenzylamide)-8-[(2,5-dimethylphenyl)amide]
[196] 1-oxa-2,8-diazaspiro[4.5]dec-2-ene-3,8-dicarboxylic acid-3-(3,4-dimethoxybenzylamide)-8-[(2-ethyl-6-methylphenyl)amide]
[197] 1-oxa-2,8-diazaspiro[4.5]dec-2-ene-3,8-dicarboxylic acid-3-(4-chlorobenzylamide)-8-[(2-ethyl-6-methylphenyl)amide]
[198] 1-oxa-2,8-diazaspiro[4.5]dec-2-ene-3,8-dicarboxylic acid-3-(3,4-dimethoxybenzylamide)-8-[(4-methoxy-2-methylphenyl)amide]
[199] 1-oxa-2,8-diazaspiro[4.5]dec-2-ene-3,8-dicarboxylic acid-3-(4-chlorobenzylamide)-8-[(4-methoxy-2-methylphenyl)amide]
[200] 1-oxa-2,8-diazaspiro[4.5]dec-2-ene-3,8-dicarboxylic acid-3-(3,4-dimethoxybenzylamide)-8-[(2-methoxy-5-methylphenyl)amide]
[201] 1-oxa-2,8-diazaspiro[4.5]dec-2-ene-3,8-dicarboxylic acid-3-(4-chlorobenzylamide)-8-[(2-methoxy-5-methylphenyl)amide]
[202] 1-oxa-2,8-diazaspiro[4.5]dec-2-ene-3,8-dicarboxylic acid-3-(3,4-dimethoxybenzylamide)-8-[(2,4,5-trimethylphenyl)amide]
[203] 1-oxa-2,8-diazaspiro[4.5]dec-2-ene-3,8-dicarboxylic acid-3-(4-chlorobenzylamide)-8-[(2,4,5-trimethylphenyl)amide]
[204] 1-oxa-2,8-diazaspiro[4.5]dec-2-ene-3,8-dicarboxylic acid-3-(3,4-dimethoxybenzylamide)-8-[(2,4,6-trimethylphenyl)amide]
[205] 1-oxa-2,8-diazaspiro[4.5]dec-2-ene-3,8-dicarboxylic acid-3-(4-chlorobenzylamide)-8-[(2,4,6-trimethylphenyl)amide]
[206] 1-oxa-2,8-diazaspiro[4.5]dec-2-ene-3,8-dicarboxylic acid-8-[(2-bromoethyl)amide]-3-(3,4-dimethoxybenzylamide)
[207] 1-oxa-2,8-diazaspiro[4.5]dec-2-ene-3,8-dicarboxylic acid-8-[(2-bromoethyl)amide]-3-(4-chlorobenzylamide)
[208] 1-oxa-2,8-diazaspiro[4.5]dec-2-ene-3,8-dicarboxylic acid-8-[(4-butylphenyl)amide]-3-(3,4-dimethoxybenzylamide)
[209] 1-oxa-2,8-diazaspiro[4.5]dec-2-ene-3,8-dicarboxylic acid-8-[(4-butylphenyl)amide]-3-(4-chlorobenzylamide)
[210] 2-{[3-(4-chlorobenzylcarbamoyl)-1-oxa-2,8-diazaspiro[4.5]dec-2-ene-8-carbonyl]amino}benzoic acid methyl ester
[211] 1-oxa-2,8-diazaspiro[4.5]dec-2-ene-3,8-dicarboxylic acid-8-biphenyl-2-ylamide-3-(3,4-dimethoxybenzylamide)
[212] 1-oxa-2,8-diazaspiro[4.5]dec-2-ene-3,8-dicarboxylic acid-8-biphenyl-2-ylamide-3-(4-chlorobenzylamide)
[213] 1-oxa-2,8-diazaspiro[4.5]dec-2-ene-3,8-dicarboxylic acid-8-[(2,6-dichlorophenyl)amide]-3-(3,4-dimethoxybenzylamide)
[214] 1-oxa-2,8-diazaspiro[4.5]dec-2-ene-3,8-dicarboxylic acid-3-(4-chlorobenzylamide)-8-[(2,6-dichlorophenyl)amide]
[215] 3-{[3-(3,4-dimethoxybenzylcarbamoyl)-1-oxa-2,8-diazaspiro[4.5]dec-2-ene-8-carbonyl]amino}benzoic acid ethyl ester
[216] 3-{[3-(4-chlorobenzylcarbamoyl)-1-oxa-2,8-diazaspiro[4.5]dec-2-ene-8-carbonyl]amino}benzoic acid ethyl ester
[217] 4-{[3-(3,4-dimethoxybenzylcarbamoyl)-1-oxa-2,8-diazaspiro[4.5]dec-2-ene-8-carbonyl]amino}benzoic acid ethyl ester
[218] 4-{[3-(4-chlorobenzylcarbamoyl)-1-oxa-2,8-diazaspiro[4.5]dec-2-ene-8-carbonyl]amino}benzoic acid ethyl ester
[219] 1-oxa-2,8-diazaspiro[4.5]dec-2-ene-3,8-dicarboxylic acid-3-(4-chlorobenzylamide)-8-naphthalen-1-ylamide
[220] 3-[4-(3-chloropyridin-2-yl)piperazine-1-carbonyl]-1-oxa-2,8-diazaspiro[4.5]dec-2-ene-8-thiocarboxylic acid-(2-chloro-5-trifluoromethylphenyl)amide
[221] 8-(4-chloro-3-trifluoromethylphenylthiocarbamoyl)-1-oxa-2,8-diazaspiro[4.5]dec-2-ene-3-carboxylic acid-3,4-dimethoxybenzylamide
[222] 8-(3,5-bis-trifluoromethylphenylthiocarbamoyl)-1-oxa-2,8-diazaspiro[4.5]dec-2-ene-3-carboxylic acid-3,4-dimethoxybenzylamide
[223] 8-(3,5-bis-trifluoromethylphenylthiocarbamoyl)-1-oxa-2,8-diazaspiro[4.5]dec-2-ene-3-carboxylic acid-4-chlorobenzylamide
[224] 3-[4-(3-chloropyridin-2-yl)piperazine-1-carbonyl]-1-oxa-2,8-diazaspiro[4.5]dec-2-ene-8-thiocarboxylic acid-(3,5-bis-trifluoromethylphenyl)amide
[225] 8 -cyclododecylthiocarbamoyl-1-oxa-2,8-diazaspiro[4.5]dec-2-ene-3-carboxylic acid-3,4-dimethoxybenzylamide
[226] 8-benzylthiocarbamoyl-1-oxa-2,8-diazaspiro[4.5]dec-2-ene-3-carboxylic acid-3,4-dimethoxybenzylamide
[227] 8-benzylthiocarbamoyl-1-oxa-2,8-diazaspiro[4.5]dec-2-ene-3-carboxylic acid-4-chlorobenzylamide
[228] 3-[4-(3-trifluoromethylpyridin-2-yl)piperazine-1-carbonyl]-1-oxa-2,8-diazaspiro[4.5]dec-2-ene-8-carboxylic acid-(4-tert-butylphenyl)amide
[229] 3-[4-(3-chloropyridin-2-yl)piperazine-1-carbonyl]-1-oxa-2,8-diazaspiro[4.5]dec-2-ene-8-carboxylic acid-(4-pentafluorosulphanylphenyl)amide,
[230] N3-((5-methylfuran-2-yl)methyl)-N-8-(4-(trifluoromethyl)phenyl)-1-oxa-2,8-diazaspiro[4.5]dec-2-ene-3,8-dicarboxamide,
[231] N8-(4-methoxyphenyl)-N-3-((5-methylfuran-2-yl)methyl)-1-oxa-2,8-diazaspiro[4.5]dec-2-ene-3,8-dicarboxamide,
[232] N8-(4-chlorophenyl)-N-3-((5-methylfuran-2-yl)methyl)-1-oxa-2,8-diazaspiro[4.5]dec-2-ene-3,8-dicarboxamide,
[233] N3-(4-(3-chloropyridin-2-yl)piperazin-1-yl)-N-8-(3,4-dichlorophenyl)-1-oxa-2,8-diazaspiro[4.5]dec-2-ene-3,8-dicarboxamide,
[234] N3-(4-(3-chloropyridin-2-yl)piperazin-1-yl)-N-8-(4-methoxyphenyl)-1-oxa-2,8-diazaspiro[4.5]dec-2-ene-3,8-dicarboxamide,
[235] N8-(4-chlorobenzyl)-N-3-((5-methylfuran-2-yl)methyl)-1-oxa-2,8-diazaspiro[4.5]dec-2-ene-3,8-dicarboxamide,
[236] N8-(3,4-dichlorobenzyl)-N-3-((5-methylfuran-2-yl)methyl)-1-oxa-2,8-diazaspiro[4.5]dec-2-ene-3,8-dicarboxamide,
[237] N3-(4-chlorobenzyl)-N-8-(3,4-dichlorobenzyl)-1-oxa-2,8-diazaspiro[4.5]dec-2-ene-3,8-dicarboxamide,
[238] N8-(3,4-dichlorobenzyl)-N-3-(4-hydroxy-3-methoxybenzyl)-1-oxa-2,8-diazaspiro[4.5]dec-2-ene-3,8-dicarboxamide,
[239] N3-(4-tert-butylbenzyl)-N-8-(3,4-dichlorobenzyl)-1-oxa-2,8-diazaspiro[4.5]dec-2-ene-3,8-dicarboxamide,
[240] N8-(3,4-dichlorobenzyl)-N-3-(4-(trifluoromethyl)benzyl)-1-oxa-2,8-diazaspiro[4.5]dec-2-ene-3,8-dicarboxamide,
[241] 3-(4-(3-chloropyridin-2-yl)piperazine-1-carbonyl)-N-(3,4-dichlorobenzyl)-1-oxa-2,8-diazaspiro[4.5]dec-2-ene-8-carboxamide,
[242] N3-(4-(3-chloropyridin-2-yl)piperazin-1-yl)-N-8-(3,4-dichlorobenzyl)-1-oxa-2,8-diazaspiro[4.5]dec-2-ene-3,8-dicarboxamide,
[243] N8-(3,4-dichlorophenyl)-N-3-((5-methylfuran-2-yl)methyl)-1-oxa-2,8-diazaspiro[4.5]dec-2-ene-3,8-dicarboxamide,
[244] N8-(4-chlorophenyl)-N-3-(4-hydroxy-3-methoxybenzyl)-1-oxa-2,8-diazaspiro[4.5]dec-2-ene-3,8-dicarboxamide,
[245] N8-(3,4-dichlorophenyl)-N-3-(4-hydroxy-3-methoxybenzyl)-1-oxa-2,8-diazaspiro[4.5]dec-2-ene-3,8-dicarboxamide,
[246] N3-(4-chlorobenzyl)-N-8-(3,4-dichlorophenyl)-1-oxa-2,8-diazaspiro[4.5]dec-2-ene-3,8-dicarboxamide,
[247] N3-(4-chlorobenzyl)-N-8-(4-chlorophenyl)-1-oxa-2,8-diazaspiro[4.5]dec-2-ene-3,8-dicarboxamide,
[248] N3-(4-hydroxy-3-methoxybenzyl)-N-8-(4-(trifluoromethyl)phenyl)-1-oxa-2,8-diazaspiro[4.5]dec-2-ene-3,8-dicarboxamide,
[249] N3-(4-hydroxy-3-methoxybenzyl)-N-8-(4-methoxyphenyl)-1-oxa-2,8-diazaspiro[4.5]dec-2-ene-3,8-dicarboxamide,
[250] N3-(4-chlorobenzyl)-N-8-(4-(trifluoromethyl)phenyl)-1-oxa-2,8-diazaspiro[4.5]dec-2-ene-3,8-dicarboxamide,
[251] N8-(3,4-dichlorobenzyl)-N-3-(3,4-dimethoxybenzyl)-1-oxa-2,8-diazaspiro[4.5]dec-2-ene-3,8-dicarboxamide,
[252] N3-(3,4-dimethoxybenzyl)-N-8-(4-(trifluoromethyl)phenyl)-1-oxa-2,8-diazaspiro[4.5]dec-2-ene-3,8-dicarboxamide,
[253] 3-[4-(3-trifluoromethylpyridin-2-yl)piperazine-1-carbonyl]-1-oxa-2,8-diazaspiro[4.5]dec-2-ene-8-carboxylic acid-(4-pentafluorosulphanylphenyl)amide,
[254] N-(4-tert-butylphenyl)-3-(4-(3-chloropyridin-2-yl)-2-methylpiperazine-1-carbonyl)-1-oxa-2,8-diazaspiro[4.5]dec-2-ene-8-carboxamide and
[255] N-(4-tert-butylbenzyl)-3-(4-(3-chloropyridin-2-yl)piperazine-1-carbonyl)-1-oxa-2,8-diazaspiro[4.5]dec-2-ene-8-carboxamide.

79. A compound according to claim 41, wherein said compound is in the form of an isolated or purified stereoisomer.

80. A compound according to claim 41, wherein said compound is in the form of a mixture of stereoisomers in any mixing ratio.

81. A compound according to claim 80, wherein said compound is in the form of a racemic mixture.
